SEQUENCE LISTING (90 sequences)

SEQ ID NO: 1 - protein, 827 aa, Mus musculus
MASADKNGSNLPSVSGSRLQSRKPPNLSITIPPPESQAPGEQDSMLPERRKNPAYLKSVSLQEPRGRWQEGAEKRPGFRRQASLSQSIRKSTAQWFGVSGDWEGKRQNWHRRSLHHCSVHYGRLKASCQRELELPSQEVPSFQGTESPKPCKMPKIVDPLARGRAFRHPDEVDRPHAAHPPLTPGVLSLTSFTSVRSGYSHLPRRKRISVAHMSFQAAAALLKGRSVLDATGQRCRHVKRSFAYPSFLEEDAVDGADTFDSSFFSKEEMSSMPDDVFESPPLSASYFRGVPHSASPVSPDGVHIPLKEYSGGRALGPGTQRGKRIASKVKHFAFDRKKRHYGLGVVGNWLNRSYRRSISSTVQRQLESFDSHRPYFTYWLTFVHIIITLLVICTYGIAPVGFAQHVTTQLVLKNRGVYESVKYIQQENFWIGPSSIDLIHLGAKFSPCIRKDQQIEQLVRRERDIERTSGCCVQNDRSGCIQTLKKDCSETLATFVKWQNDTGPSDKSDLSQKQPSAVVCHQDPRTCEEPASSGAHIWPDDITKWPICTEQAQSNHTGLLHIDCKIKGRPCCIGTKGSCEITTREYCEFMHGYFHEDATLCSQVHCLDKVCGLLPFLNPEVPDQFYRIWLSLFLHAGIVHCLVSVVFQMTILRDLEKLAGWHRISIIFILSGITGNLASAIFLPYRAEVGPAGSQFGLLACLFVELFQSWQLLERPWKAFFNLSAIVLFLFICGLLPWIDNIAHIFGFLSGMLLAFAFLPYITFGTSDKYRKRALILVSLLVFAGLFASLVLWLYIYPINWPWIEYLTCFPFTSRFCEKYELDQVLH

SEQ ID NO: 2 - protein, 559 aa, Mus musculus
MSSMPDDVFESPPLSASYFRGVPHSASPVSPDGVHIPLKEYSGGRALGPGTQRGKRIASKVKHFAFDRKKRHYGLGVVGNWLNRSYRRSISSTVQRQLESFDSHRPYFTYWLTFVHIIITLLVICTYGIAPVGFAQHVTTQLVLKNRGVYESVKYIQQENFWIGPSSIDLIHLGAKFSPCIRKDQQIEQLVRRERDIERTSGCCVQNDRSGCIQTLKKDCSETLATFVKWQNDTGPSDKSDLSQKQPSAVVCHQDPRTCEEPASSGAHIWPDDITKWPICTEQAQSNHTGLLHIDCKIKGRPCCIGTKGSCEITTREYCEFMHGYFHEDATLCSQVHCLDKVCGLLPFLNPEVPDQFYRIWLSLFLHAGIVHCLVSVVFQMTILRDLEKLAGWHRISIIFILSGITGNLASAIFLPYRAEVGPAGSQFGLLACLFVELFQSWQLLERPWKAFFNLSAIVLFLFICGLLPWIDNIAHIFGFLSGMLLAFAFLPYITFGTSDKYRKRALILVSLLVFAGLFASLVLWLYIYPINWPWIEYLTCFPFTSRFCEKYELDQVLH

SEQ ID NO: 3 - protein, 856 aa, Mus musculus
MSEARRDSTSSLQRKKPPWLKLDIPAAVPPAAEEPSFLQPLRRQAFLRSVSMPAETARVPSPHHEPRRLVLQRQTSITQTIRRGTADWFGVSKDSDSTQKWQRKSIRHCSQRYGKLKPQVIRELDLPSQDNVSLTSTETPPPLYVGPCQLGMQKIIDPLARGRAFRMADDTADGLSAPHTPVTPGAASLCSFSSSRSGFNRLPRRRKRESVAKMSFRAAAALVKGRSIRDGTLRRGQRRSFTPASFLEEDMVDFPDELDTSFFAREGVLHEEMSTYPDEVFESPSEAALKDWEKAPDQADLTGGALDRSELERSHLMLPLERGWRKQKEGGPLAPQPKVRLRQEVVSAAGPRRGQRIAVPVRKLFAREKRPYGLGMVGRLTNRTYRKRIDSYVKRQIEDMDDHRPFFTYWLTFVHSLVTILAVCIYGIAPVGFSQHETVDSVLRKRGVYENVKYVQQENFWIGPSSEALIHLGAKFSPCMRQDPQVHSFILAAREREKHSACCVRNDRSGCVQTSKEECSSTLAVWVKWPVHPSAPDLAGNKRQFGSVCHQDPRVCDEPSSEDPHEWPEDITKWPICTKSSAGNHTNHPHMDCVITGRPCCIGTKGRCEITSREYCDFMRGYFHEEATLCSQVHCMDDVCGLLPFLNPEVPDQFYRLWLSLFLHAGILHCLVSVCFQMTVLRDLEKLAGWHRIAIIYLLSGITGNLASAIFLPYRAEVGPAGSQFGILACLFVELFQSWQILARPWRAFFKLLAVVLFLFAFGLLPWIDNFAHISGFVSGLFLSFAFLPYISFGKFDLYRKRCQIIIFQVVFLGLLAGLVVLFYFYPVRCEWCEFLTCIPFTDKFCEKYELDAQLH

SEQ ID NO: 4 - protein, 584 aa, Mus musculus
MSTYPDEVFESPSEAALKDWEKAPDQADLTGGALDRSELERSHLMLPLERGWRKQKEGGPLAPQPKVRLRQEVVSAAGPRRGQRIAVPVRKLFAREKRPYGLGMVGRLTNRTYRKRIDSYVKRQIEDMDDHRPFFTYWLTFVHSLVTILAVCIYGIAPVGFSQHETVDSVLRKRGVYENVKYVQQENFWIGPSSEALIHLGAKFSPCMRQDPQVHSFILAAREREKHSACCVRNDRSGCVQTSKEECSSTLAVWVKWPVHPSAPDLAGNKRQFGSVCHQDPRVCDEPSSEDPHEWPEDITKWPICTKSSAGNHTNHPHMDCVITGRPCCIGTKGRCEITSREYCDFMRGYFHEEATLCSQVHCMDDVCGLLPFLNPEVPDQFYRLWLSLFLHAGILHCLVSVCFQMTVLRDLEKLAGWHRIAIIYLLSGITGNLASAIFLPYRAEVGPAGSQFGILACLFVELFQSWQILARPWRAFFKLLAVVLFLFAFGLLPWIDNFAHISGFVSGLFLSFAFLPYISFGKFDLYRKRCQIIIFQVVFLGLLAGLVVLFYFYPVRCEWCEFLTCIPFTDKFCEKYELDAQLH

SEQ ID NO: 5 - protein, 856 aa, Homo sapiens
MASADKNGGSVSSVSSSRLQSRKPPNLSITIPPPEKETQAPGEQDSMLPEGFQNRRLKKSQPRTWAAHTTACPPSFLPKRKNPAYLKSVSLQEPRSRWQESSEKRPGFRRQASLSQSIRKGAAQWFGVSGDWEGQRQQWQRRSLHHCSMRYGRLKASCQRDLELPSQEAPSFQGTESPKPCKMPKIVDPLARGRAFRHPEEMDRPHAPHPPLTPGVLSLTSFTSVRSGYSHLPRRKRMSVAHMSLQAAAALLKGRSVLDATGQRCRVVKRSFAFPSFLEEDVVDGADTFDSSFFSKEEMSSMPDDVFESPPLSASYFRGIPHSASPVSPDGVQIPLKEYGRAPVPGPRRGKRIASKVKHFAFDRKKRHYGLGVVGNWLNRSYRRSISSTVQRQLESFDSHRPYFTYWLTFVHVIITLLVICTYGIAPVGFAQHVTTQLVLRNKGVYESVKYIQQENFWVGPSSIDLIHLGAKFSPCIRKDGQIEQLVLRERDLERDSGCCVQNDHSGCIQTQRKDCSETLATFVKWQDDTGPPMDKSDLGQKRTSGAVCHQDPRTCEEPASSGAHIWPDDITKWPICTEQARSNHTGFLHMDCEIKGRPCCIGTKGSCEITTREYCEFMHGYFHEEATLCSQVHCLDKVCGLLPFLNPEVPDQFYRLWLSLFLHAGVVHCLVSVVFQMTILRDLEKLAGWHRIAIIFILSGITGNLASAIFLPYRAEVGPAGSQFGLLACLFVELFQSWPLLERPWKAFLNLSAIVLFLFICGLLPWIDNIAHIFGFLSGLLLAFAFLPYITFGTSDKYRKRALILVSLLAFAGLFAALVLWLYIYPINWPWIEHLTCFPFTSRFCEKYELDQVLH

SEQ ID NO: 6 - protein, 558 aa, Homo sapiens
MSSMPDDVFESPPLSASYFRGIPHSASPVSPDGVQIPLKEYGRAPVPGPRRGKRIASKVKHFAFDRKKRHYGLGVVGNWLNRSYRRSISSTVQRQLESFDSHRPYFTYWLTFVHVIITLLVICTYGIAPVGFAQHVTTQLVLRNKGVYESVKYIQQENFWVGPSSIDLIHLGAKFSPCIRKDGQIEQLVLRERDLERDSGCCVQNDHSGCIQTQRKDCSETLATFVKWQDDTGPPMDKSDLGQKRTSGAVCHQDPRTCEEPASSGAHIWPDDITKWPICTEQARSNHTGFLHMDCEIKGRPCCIGTKGSCEITTREYCEFMHGYFHEEATLCSQVHCLDKVCGLLPFLNPEVPDQFYRLWLSLFLHAGVVHCLVSVVFQMTILRDLEKLAGWHRIAIIFILSGITGNLASAIFLPYRAEVGPAGSQFGLLACLFVELFQSWPLLERPWKAFLNLSAIVLFLFICGLLPWIDNIAHIFGFLSGLLLAFAFLPYITFGTSDKYRKRALILVSLLAFAGLFAALVLWLYIYPINWPWIEHLTCFPFTSRFCEKYELDQVLH

SEQ ID NO: 7 - protein, 855 aa, Homo sapiens
MSEARRDSTSSLQRKKPPWLKLDIPSAVPLTAEEPSFLQPLRRQAFLRSVSMPAETAHISSPHHELRRPVLQRQTSITQTIRRGTADWFGVSKDSDSTQKWQRKSIRHCSQRYGKLKPQVLRELDLPSQDNVSLTSTETPPPLYVGPCQLGMQKIIDPLARGRAFRVADDTAEGLSAPHTPVTPGAASLCSFSSSRSGFHRLPRRRKRESVAKMSFRAAAALMKGRSVRDGTFRRAQRRSFTPASFLEEDTTDFPDELDTSFFAREGILHEELSTYPDEVFESPSEAALKDWEKAPEQADLTGGALDRSELERSHLMLPLERGWRKQKEGAAAPQPKVRLRQEVVSTAGPRRGQRIAVPVRKLFAREKRPYGLGMVGRLTNRTYRKRIDSFVKRQIEDMDDHRPFFTYWLTFVHSLVTILAVCIYGIAPVGFSQHETVDSVLRNRGVYENVKYVQQENFWIGPSSEALIHLGAKFSPCMRQDPQVHSFIRSAREREKHSACCVRNDRSGCVQTSEEECSSTLAVWVKWPIHPSAPELAGHKRQFGSVCHQDPRVCDEPSSEDPHEWPEDITKWPICTKNSAGNHTNHPHMDCVITGRPCCIGTKGRCEITSREYCDFMRGYFHEEATLCSQVHCMDDVCGLLPFLNPEVPDQFYRLWLSLFLHAGILHCLVSICFQMTVLRDLEKLAGWHRIAIIYLLSGVTGNLASAIFLPYRAEVGPAGSQFGILACLFVELFQSWQILARPWRAFFKLLAVVLFLFTFGLLPWIDNFAHISGFISGLFLSFAFLPYISFGKFDLYRKRCQIIIFQVVFLGLLAGLVVLFYVYPVRCEWCEFLTCIPFTDKFCEKYELDAQLH

SEQ ID NO: 8 - protein, 539 aa, Homo sapiens
MLPLERGWRKQKEGAAAPQPKVRLRQEVVSTAGPRRGQRIAVPVRKLFAREKRPYGLGMVGRLTNRTYRKRIDSFVKRQIEDMDDHRPFFTYWLTFVHSLVTILAVCIYGIAPVGFSQHETVDSVLRNRGVYENVKYVQQENFWIGPSSEALIHLGAKFSPCMRQDPQVHSFIRSAREREKHSACCVRNDRSGCVQTSEEECSSTLAVWVKWPIHPSAPELAGHKRQFGSVCHQDPRVCDEPSSEDPHEWPEDITKWPICTKNSAGNHTNHPHMDCVITGRPCCIGTKGRCEITSREYCDFMRGYFHEEATLCSQVHCMDDVCGLLPFLNPEVPDQFYRLWLSLFLHAGILHCLVSICFQMTVLRDLEKLAGWHRIAIIYLLSGVTGNLASAIFLPYRAEVGPAGSQFGILACLFVELFQSWQILARPWRAFFKLLAVVLFLFTFGLLPWIDNFAHISGFISGLFLSFAFLPYISFGKFDLYRKRCQIIIFQVVFLGLLAGLVVLFYVYPVRCEWCEFLTCIPFTDKFCEKYELDAQLH

SEQ ID NO: 9 - DNA, 18 bp, Artificial Sequence
  PCR PRIMER
aaaaccctccacccttcc

SEQ ID NO: 10 - DNA, 20 bp, Artificial Sequence
  PCR PRIMER
cggggaacaactctgaatcc

SEQ ID NO: 11 - DNA, 20 bp, Artificial Sequence
  PCR PRIMER
agagccactttgggtctgtc

SEQ ID NO: 12 - DNA, 20 bp, Artificial Sequence
  PCR PRIMER
acgcaacgttctgtcaaaag

SEQ ID NO: 13 - DNA, 27 bp, Artificial Sequence
  PCR PRIMER
tgtattgaacatgtattatgaagttgg

SEQ ID NO: 14 - DNA, 20 bp, Artificial Sequence
  PCR PRIMER
caactgtgctgtgtgcatcc

SEQ ID NO: 15 - DNA, 20 bp, Artificial Sequence
  PCR PRIMER
ccttgtgccttagcaaatgt

SEQ ID NO: 16 - DNA, 23 bp, Artificial Sequence
  PCR PRIMER
agacacacacatcaacaataggg

SEQ ID NO: 17 - DNA, 21 bp, Artificial Sequence
  PCR PRIMER
tccatcaccatcaacacacag

SEQ ID NO: 18 - DNA, 22 bp, Artificial Sequence
  PCR PRIMER
ccctgaaattccataaggacag

SEQ ID NO: 19 - DNA, 22 bp, Artificial Sequence
  PCR PRIMER
tcaatcaagtccaagtaatgcc

SEQ ID NO: 20 - DNA, 20 bp, Artificial Sequence
  PCR PRIMER
attccacatgctgttgttcc

SEQ ID NO: 21 - DNA, 20 bp, Artificial Sequence
  PCR PRIMER
tcacacttggtatccatcgc

SEQ ID NO: 22 - DNA, 22 bp, Artificial Sequence
  PCR PRIMER
aaaagaaataagcccatggttc

SEQ ID NO: 23 - DNA, 20 bp, Artificial Sequence
  PCR PRIMER
ctagtaagtcctcgcgtgcc

SEQ ID NO: 24 - DNA, 20 bp, Artificial Sequence
  PCR PRIMER
gtcctatgacaaatgcaccg

SEQ ID NO: 25 - DNA, 20 bp, Artificial Sequence
  PCR PRIMER
aacgaaagttgctttcctgg

SEQ ID NO: 26 - DNA, 25 bp, Artificial Sequence
  PCR PRIMER
ttcattggcatgtacttatattgac

SEQ ID NO: 27 - DNA, 21 bp, Artificial Sequence
  PCR PRIMER
gctatacagccagagtcccag

SEQ ID NO: 28 - DNA, 20 bp, Artificial Sequence
  PCR PRIMER
cagaagaggcgaggaaagtg

SEQ ID NO: 29 - DNA, 22 bp, Artificial Sequence
  PCR PRIMER
ggtttcttatattgcagtgggg

SEQ ID NO: 30 - DNA, 20 bp, Artificial Sequence
  PCR PRIMER
cgcagattccactttgttcc

SEQ ID NO: 31 - DNA, 22 bp, Artificial Sequence
  PCR PRIMER
tgtctgggcattttacataagc

SEQ ID NO: 32 - DNA, 20 bp, Artificial Sequence
  PCR PRIMER
tgtgccaagccataattcag

SEQ ID NO: 33 - DNA, 20 bp, Artificial Sequence
  PCR PRIMER
tgacattcctgcgagcttag

SEQ ID NO: 34 - DNA, 19 bp, Artificial Sequence
  PCR PRIMER
caagatctggttagcgccc

SEQ ID NO: 35 - DNA, 22 bp, Artificial Sequence
  PCR PRIMER
tctgaagacaaatttgaacccc

SEQ ID NO: 36 - DNA, 23 bp, Artificial Sequence
  PCR PRIMER
ttccatgggtatggataatgaag

SEQ ID NO: 37 - DNA, 19 bp, Artificial Sequence
  PCR PRIMER
ggtgcccagattcaaaatg

SEQ ID NO: 38 - DNA, 20 bp, Artificial Sequence
  PCR PRIMER
tagtgcaccagaatgccttg

SEQ ID NO: 39 - DNA, 19 bp, Artificial Sequence
  PCR PRIMER
aagtgccaagggaccaatc

SEQ ID NO: 40 - DNA, 20 bp, Artificial Sequence
  PCR PRIMER
taccccaaggttcttgcatc

SEQ ID NO: 41 - DNA, 24 bp, Artificial Sequence
  PCR PRIMER
gaaaacaggcgttttagatattgg

SEQ ID NO: 42 - DNA, 21 bp, Artificial Sequence
  PCR PRIMER
cacggagtggatctactggac

SEQ ID NO: 43 - DNA, 25 bp, Artificial Sequence
  PCR PRIMER
cactatactgagaaccaaagcaacc

SEQ ID NO: 44 - DNA, 20 bp, Artificial Sequence
  PCR PRIMER
aagtgctgtttatgctcccc

SEQ ID NO: 45 - DNA, 21 bp, Artificial Sequence
  PCR PRIMER
caaatttgtcacgtttggttc

SEQ ID NO: 46 - DNA, 20 bp, Artificial Sequence
  PCR PRIMER
ccgacactactttcaagccc

SEQ ID NO: 47 - DNA, 20 bp, Artificial Sequence
  PCR PRIMER
tccagcgggtctataaaagc

SEQ ID NO: 48 - DNA, 20 bp, Artificial Sequence
  PCR PRIMER
gacgtttggggaatgtcaag

SEQ ID NO: 49 - DNA, 21 bp, Artificial Sequence
  PCR PRIMER
tgtctacacagcaatgctcac

SEQ ID NO: 50 - DNA, 20 bp, Artificial Sequence
  PCR PRIMER
ctttgcagaggtaaaacccg

SEQ ID NO: 51 - DNA, 20 bp, Artificial Sequence
  PCR PRIMER
aaaagagaacgtgggcagtc

SEQ ID NO: 52 - DNA, 23 bp, Artificial Sequence
  PCR PRIMER
ccttggtttacacactggaaaag

SEQ ID NO: 53 - DNA, 23 bp, Artificial Sequence
  PCR PRIMER
gtatgtttgagatgcagaaaacc

SEQ ID NO: 54 - DNA, 27 bp, Artificial Sequence
  PCR PRIMER
tgacagtagagtctgataggaatactg

SEQ ID NO: 55 - DNA, 20 bp, Artificial Sequence
  PCR PRIMER
gtcacaggaggaaagtcccc

SEQ ID NO: 56 - DNA, 23 bp, Artificial Sequence
  PCR PRIMER
cttcttttgccttgagataagtg

SEQ ID NO: 57 - DNA, 27 bp, Artificial Sequence
  PCR PRIMER
tggtggttttctataagaatatttagc

SEQ ID NO: 58 - DNA, 22 bp, Artificial Sequence
  PCR PRIMER
aagcccttgtaaatgattgtcc

SEQ ID NO: 59 - DNA, 20 bp, Artificial Sequence
  PCR PRIMER
aaggttcacaggactccagc

SEQ ID NO: 60 - DNA, 18 bp, Artificial Sequence
  PCR PRIMER
aagtggtgtgcctctccg

SEQ ID NO: 61 - DNA, 21 bp, Artificial Sequence
  PCR PRIMER
aaaaggattaagcatttgggg

SEQ ID NO: 62 - DNA, 22 bp, Artificial Sequence
  PCR PRIMER
tgggtcatgacactaaaaggac

SEQ ID NO: 63 - DNA, 20 bp, Artificial Sequence
  PCR PRIMER
tttccaatgctgtcaaaccc

SEQ ID NO: 64 - DNA, 21 bp, Artificial Sequence
  PCR PRIMER
gaatcaatgaataaaacgggg

SEQ ID NO: 65 - DNA, 21 bp, Artificial Sequence
  PCR PRIMER
tgggaagaggagaaaagcaag

SEQ ID NO: 66 - DNA, 26 bp, Artificial Sequence
  PCR PRIMER
gaaaacagtgctaattacttccaaag

SEQ ID NO: 67 - DNA, 20 bp, Artificial Sequence
  PCR PRIMER
attagacatggtgaagcccc

SEQ ID NO: 68 - DNA, 23 bp, Artificial Sequence
  PCR PRIMER
tttgcatactgacaggaactcac

SEQ ID NO: 69 - DNA, 19 bp, Artificial Sequence
  PCR PRIMER
tgctgagcagacagtcacg

SEQ ID NO: 70 - DNA, 17 bp, Artificial Sequence
  PCR PRIMER
ttgggctcatgtgcgag

SEQ ID NO: 71 - DNA, 20 bp, Artificial Sequence
  PCR PRIMER
tgctttggtcagtcaaccag

SEQ ID NO: 72 - DNA, 20 bp, Artificial Sequence
  PCR PRIMER
ttttcccactcttgtggtcc

SEQ ID NO: 73 - DNA, 20 bp, Artificial Sequence
  PCR PRIMER
ttcccattggctgctgtaag

SEQ ID NO: 74 - DNA, 20 bp, Artificial Sequence
  PCR PRIMER
ccaactgaaccacactttgc

SEQ ID NO: 75 - DNA, 21 bp, Artificial Sequence
  PCR PRIMER
tccatgaagtcaatcctgaag

SEQ ID NO: 76 - DNA, 20 bp, Artificial Sequence
  PCR PRIMER
gaggcaggcaaatctctgtg

SEQ ID NO: 77 - DNA, 20 bp, Artificial Sequence
  PCR PRIMER
tggttggttggttggttagg

SEQ ID NO: 78 - DNA, 20 bp, Artificial Sequence
  PCR PRIMER
tgaagcacaaaagccaactc

SEQ ID NO: 79 - DNA, 20 bp, Artificial Sequence
  PCR PRIMER
cggtggaaccaatgagaact

SEQ ID NO: 80 - DNA, 20 bp, Artificial Sequence
  PCR PRIMER
ctcaccctcctagcatgagc

SEQ ID NO: 81 - DNA, 20 bp, Artificial Sequence
  PCR PRIMER
cggtggaaccaatgagaact

SEQ ID NO: 82 - DNA, 20 bp, Artificial Sequence
  PCR PRIMER
gtcgtagtcccctgtggaga

SEQ ID NO: 83 - DNA, 20 bp, Artificial Sequence
  PCR PRIMER
cggtggaaccaatgagaact

SEQ ID NO: 84 - DNA, 20 bp, Artificial Sequence
  PCR PRIMER
ttgcctccctttttcttcct

SEQ ID NO: 85 - DNA, 20 bp, Artificial Sequence
  PCR PRIMER
cggtggaaccaatgagaact

SEQ ID NO: 86 - DNA, 20 bp, Artificial Sequence
  PCR PRIMER
ccacaccgttcaccaaagta

SEQ ID NO: 87 - DNA, 20 bp, Artificial Sequence
  PCR PRIMER
gttgctgcagagaccgagac

SEQ ID NO: 88 - DNA, 20 bp, Artificial Sequence
  PCR PRIMER
gtcgtagtcccctgtggaga

SEQ ID NO: 89 - DNA, 20 bp, Artificial Sequence
  PCR PRIMER
ctggcagtgaactctccaca

SEQ ID NO: 90 - DNA, 20 bp, Artificial Sequence
  PCR PRIMER
ccacaccgttcaccaaagta

(19) United States
(12) Patent Application Publication
(10) Pub. No.: US 2017/0241986 A1
(43) Pub. Date: Aug. 24, 2017

(54) METHODS FOR IDENTIFYING COMPOUNDS THAT ALTER THE ACTIVITY OF IRHOM POLYPEPTIDES AND USE THEREOF

(71) Applicant: The Jackson Laboratory, Bar Harbor, ME (US)

(72) Inventors: Vishnu Hosur, Bar Harbor, ME (US); Leonard D. Shultz, Bar Harbor, ME (US)

(21) Appl. No.: 15/310,022

(22) PCT Filed: May 11, 2015
(86) PCT No.: PCT/US2015/030140

§ 371 Date: Nov. 9, 2016

Related U.S. Application Data
(60) Provisional application No. 61/992,152, filed on May 12, 2014.
(60) Provisional application No. 61/991,423, filed on May 9, 2014.

(51) Int. Cl. G01N 33/50

(52) U.S. Cl. CPC G01N 33/5038; G01N 33/5011; G01N 33/5041; G01N 2333/71; G01N 2333/96425; G01N 2500/10; G01N 2500/02

(57) ABSTRACT

Methods for identifying a compound that modulates activity of an iRhom polypeptide according to aspects of the present invention are described herein which include contacting a cell expressing an iRhom polypeptide with a test compound; and determining the effect of the compound on activity of the iRhom polypeptide. Detection of a decrease in proteolytic activity of the iRhom polypeptide indicates that the compound is capable of one or more of: reducing tumor growth, reducing tumor progression, treatment of cancer and promoting hair growth in a subject, and detecting an increase in proteolytic activity of the iRhom polypeptide indicates that the compound is capable of accelerating wound healing in a subject.

### REFERENCE TO RELATED APPLICATIONS

This application claims priority to U.S. Provisional Patent Application Ser. No. 61/991,423, filed May 9, 2014 and 61/992,152, filed May 12, 2014, the entire content of both of which is incorporated herein by reference.

### GRANT REFERENCE

This invention was made with government support under Grant Nos. CA034196, HLO77642, and DK057199, awarded by the National Institutes of Health. The Government has certain rights in the invention.

### FIELD OF THE INVENTION

Methods for identifying compounds that alter the activity of iRhom polypeptides and uses thereof are generally described herein. According to specific aspects, methods for identifying compounds that modulate the proteolytic activity of iRhom polypeptides, such as increasing, stimulating, reducing, inhibiting or abolishing the proteolytic activity of iRhom polypeptides are disclosed herein along with uses thereof.

### BACKGROUND OF THE INVENTION

The rhomboid proteases are a family of enzymes that exist in almost all species. Rhomboids are intramembrane serine proteases and proteolytic cleavage by Rhomboid proteases is important for cellular regulation. The active site of intramembrane proteases is buried in the lipid bilayer of cell membranes, and they cleave other transmembrane proteins within their transmembrane domains.

Inactive rhomboids (iRhoms) are highly conserved intramembrane proteins. Prior to the discovery of the present invention, it was thought that iRhoms were proteolytically inactive due to the lack of a serine residue in the putative active site.

### SUMMARY OF THE INVENTION

In one aspect, the present invention is based on the discovery that iRhoms are short-lived proteins, but dominant mutations increase their protein stability and stimulate secretion of specific EGF family ligand amphiregulin independently of metalloprotease activity. In another aspect, the invention is based on the discovery that mammalian iRhoms function in regulating an EGFR signaling event that promotes accelerated wound healing and triggers tumorigenesis. In yet another aspect, the present invention is based, at least in part, on the discovery that iRhoms have the ability to regulate EGFR signaling in parallel with metalloproteases, and may function as therapeutic targets in impaired wound healing and cancer.

In one aspect the invention provides methods for identifying a compound that activates the proteolytic activity of an iRhom polypeptide on a substrate comprising a) contacting a cell expressing an iRhom polypeptide with a test compound; and b) determining an increase in stability of the iRhom polypeptide relative to an appropriate control, wherein an increase in stability of the iRhom polypeptide indicates that the compound activates the proteolytic activity of the iRhom polypeptide on a substrate.

In another aspect, the invention provides a method for identifying a compound capable of accelerating wound healing or tissue repair in a subject comprising a) contacting a cell expressing an iRhom polypeptide and an EGFR ligand with a test compound; b) determining an increase in stability of the iRhom polypeptide relative to an appropriate control; and c) determining an increase in secretion of an EGFR ligand by the cell relative to an appropriate control, wherein an increase in stability of the iRhom polypeptide and an increase in secretion of an EGFR ligand by the cell indicates that the compound is capable of accelerating wound healing.

In another aspect, the invention provides a method for identifying a compound that inhibits the cytoplasmic domain of an iRhom polypeptide comprising a) contacting a cell expressing an iRhom polypeptide with a test compound; b) determining an increase in stability of the iRhom polypeptide relative to an appropriate control, wherein an increase in stability of the iRhom polypeptide indicates that the compound inhibited the cytoplasmic domain of the iRhom polypeptide.

In one embodiment, the method further provides that an increase in stability of the iRhom polypeptide is determined by analyzing the half-life of the iRhom polypeptide following exposure to the compound.

In some embodiments, the substrate is an EGFR ligand or an EGF-like substrate.

In another embodiment, an increase in stability of the iRhom polypeptide is determined by detecting an increase in secretion of an EGFR ligand. In another embodiment, an increase in stability of an iRhom polypeptide is determined by detecting an increase in the level of soluble EGFR ligands. In some embodiments, the EGFR ligand is selected from the group consisting of AREG, HB-EGF, TGFα and EPGN.

In another embodiment, an increase in stability of an iRhom polypeptide is determined by detecting an increase in EGFR signaling activity.

In yet another embodiment, the compound of the invention inhibits an interaction between the iRhom polypeptide and a proteasome.

In yet another embodiment, the compound inactivates the cytoplasmic domain of an iRhom polypeptide. In some embodiments, the inactivation of the cytoplasmic domain is transient.

In one embodiment, the compound of the invention cleaves and/or deletes the cytoplasmic domain of an iRhom polypeptide such that the polypeptide has proteolytic activity or altered biological activity. In some embodiments, mouse iRhom2 is cleaved between amino acid residues 1 and 268 or human iRhom2 is cleaved between amino acid residues 1 and 298. In other embodiments, mouse iRhom1 is cleaved between amino acid residues 1 and 272 or human iRhom1 is cleaved between amino acid residues 1 and 316. In another embodiment, the compound activates the peptidase domain of an iRhom polypeptide.

In one embodiment, the iRhom polypeptide is iRhom1 or iRhom2. In another embodiment, the iRhom polypeptide is a human or mouse iRhom polypeptide.

In one embodiment, the compound of the invention is selected from the group consisting of a small molecule, a peptide or a polypeptide decoy. In another embodiment, the compound is attached to a cell penetrating peptide. In another embodiment, the compound is cell-permeable. In yet another embodiment, the compound is an ubiquitin protease inhibitor.

In one embodiment, the compound of the invention accelerates migration of keratinocytes. In yet another embodiment, the compound accelerates proliferation of fibroblasts.

In one aspect, the invention provides a method for identifying a compound that inhibits the proteolytic activity of an iRhom polypeptide on a substrate comprising a) contacting a cell expressing an iRhom polypeptide with a test compound; and b) determining a decrease in secretion of a physiological target of the iRhom polypeptide relative to an appropriate control or a decrease in EGFR activity relative to an appropriate control, wherein a decrease in secretion of a physiological target of the iRhom polypeptide or a decrease in EGFR activity indicates that the compound inhibits the proteolytic activity of the iRhom polypeptide on a substrate.

In another aspect, the invention provides a method for identifying a compound capable of reducing tumor growth and/or progression or treating cancer in a subject comprising a) contacting a cell expressing an iRhom polypeptide with a test compound; and b) determining a decrease in secretion of a physiological target of the iRhom polypeptide relative to an appropriate control or a decrease in EGFR activity relative to an appropriate control, wherein a decrease in secretion of a physiological target of the iRhom polypeptide or a decrease in EGFR activity indicates that the compound is capable of reducing tumor growth and/or progression or treating cancer in the subject.

In another aspect, the invention provides a method for identifying a compound capable of promoting hair growth in a subject comprising a) contacting a cell expressing an iRhom polypeptide with a test compound; and b) determining a decrease in secretion of a physiological target of the iRhom polypeptide relative to an appropriate control or a decrease in EGFR activity relative to an appropriate control, wherein a decrease in secretion of a physiological target of the iRhom polypeptide or a decrease in EGFR activity indicates that the compound is capable of promoting hair growth in the subject.

In one embodiment, the tumor is a solid tumor. In some embodiments, the cancer is an epithelial cancer. In other embodiments, the cancer is cancer of the esophagus, lung, brain, colon, kidney, prostate, skin, liver, pancreas, stomach, uterus, ovary, breast, lymph glands or bladder.

In one embodiment, the substrate is an EGFR ligand or an EGF-like substrate.

In another embodiment, the inhibition of proteolytic activity of the iRhom polypeptide is determined by detecting a decrease in secretion of a physiological target of the iRhom polypeptide. In some embodiments, the physiological target of the iRhom polypeptide is an EGFR ligand. In one embodiment, the inhibition of proteolytic activity of the iRhom polypeptide is determined by detecting a decrease in the level of soluble EGFR ligands. In yet other embodiments, the EGFR ligand is selected from the group consisting of AREG, HB-EGF, TGFα and EPGN.

In one embodiment, the inhibition of proteolytic activity of an iRhom polypeptide is determined by detecting a decrease in EGFR activity.

In another embodiment, the compound inhibits the peptidase domain of an iRhom polypeptide.

In yet another embodiment, the compound affects the activity of an iRhom polypeptide by inactivating one or more amino acid residues in the proteolyic site of the iRhom polypeptide peptidase domain. In one embodiment, the iRhom polypeptide is mouse iRhom2 and the compound inactivates one or more amino acid residues in the proteolytic site of mouse iRhom2 selected from the group consisting of Histidine 635 on TM helix 2, Glutamine 695 on TM helix 4, Cysteine 701 on TM helix 4, and Histidine 744 on TM helix 6. In other embodiments, the iRhom polypeptide is human iRhom2 and the compound inactivates one or more amino acid residues in the proteolytic site of human iRhom2 selected from the group consisting of Histidine 664 on TM helix 2, Glutamine 724 on TM helix 4, Cysteine 730 on TM helix 4, and Histidine 773 on TM helix 6.

In one embodiment, the iRhom family member is iRhom1 or iRhom2. In other embodiments, the iRhom family member is a human or mouse iRhom family member.

In one embodiment, the compound of the invention is selected from the group consisting of a small molecule, a polypeptide decoy, an miRNA molecule, an siRNA molecule, an shRNA molecule, a dsRNA molecule, an antisense molecule, a ribozyme specific for Rhbdf2; or a polynucleotide encoding the miRNA, siRNA, shRNA, dsRNA; or a biological equivalent of each thereof. In other embodiments, the compound is attached to a cell penetrating peptide. In some embodiments, the compound is cell-permeable.

In one aspect, the invention provides an isolated polypeptide comprising a variant iRhom polypeptide.

In another aspect, the invention provides an isolated polypeptide comprising human iRhom2 with a deletion of the cytosolic N-terminal domain.

In another aspect, the invention provides an isolated polypeptide comprising mouse iRhom2 with a deletion of the cytosolic N-terminal domain.

In yet another aspect, the invention provides an isolated mouse iRhom2 polypeptide comprising a mutation at one or more amino acid residues in the proteolytic site of mouse iRhom2 selected from the group consisting of Histidine 635 on TM helix 2, Glutamine 695 on TM helix 4, Cysteine 701 on TM helix 4 and Histidine 744 on TM helix 6.

In another aspect, the invention provides an isolated human iRhom2 polypeptide comprising a mutation at one or more amino acid residues in the proteolytic site of human iRhom2 selected from the group consisting of Histidine 664 on TM helix 2, Glutamine 724 on TM helix 4, Cysteine 730 on TM helix 4 and Histidine 773 on TM helix 6.

In one embodiment, the invention provides an isolated nucleic acid molecule encoding any one of the foregoing polypeptides. In another embodiment, the invention provides a vector comprising the foregoing nucleic acid molecule. In another embodiment, the invention provides a host cell expressing the foregoing vector.

### BRIEF DESCRIPTION OF THE DRAWINGS

FIG. 1A is a graph showing quantitation of MEFs using the proliferation assay described herein; cells were seeded at the numbers indicated on the X-axis and incubated for 24 h, with the extent of fluorescence being proportional to the amount of total cellular DNA; data is shown as mean±s.d.;

FIG. 1B is a graph showing a quantitation of migration of cub/cub mcub/mcub and +/+ mcub/mcub MEFs using a scratch-wound assay described herein; the width of the scratch wound was measured at time zero (100% open), and the increase in wound closure at each time point was calculated as a percent of that original width; data is shown as mean±s.d.;

FIG. 1C is an image showing an immunoblot analysis of cub/cub mcub/mcub or +/+ mcub/mcub MEFs for various markers of EGFR signaling in which cell lysates were run in duplicate with actin serving as a loading control;

FIG. 1D shows representative images of regenerating ear tissue in 6-40-week-old female cub/cub mcub/mcub and +/+ mcub/mcub mice (n=3 per group) at 0, 7, 14, 21 and 28 days post-wounding;

FIG. 1E is a graph showing a quantification of ear hole closures shown in FIG. 1D; data is shown as mean±s.d.;

FIG. 1F shows images of cross-sections of ears from cub/cub mcub/mcub and +/+ mcub/mcub mice at 0, 7 and 14 days post-wounding stained with Hematoxylin/Eosin (original magnification, ×10); note the undifferentiated and thickened epidermis (E) (10-12 nucleated layers) and the extensive degree of proliferation (M) in the ears of cub/cub mcub/mcub mice; the dotted line indicates the site of excision;

FIG. 2A is a diagram showing that the cub mutation is a 12,681 bp deletion in the mouse Rhbdf2 gene in which the deletion starts midway between exons 1 and 2 and encompasses exons 2-6, ending shortly after exon 6;

FIG. 2B shows images of results from a reverse-transcriptase PCR on MEFs from +/+ mcub/mcub, +/cub mcub/mcub and cub/cub mcub/mcub mice using primers against exons 2, 5, 12 and 19; ntc represents the no template control;

FIG. 2C is a graph showing results from a qPCR on cDNA extracted from skin tissues using TaqMan gene expression assays against the indicated exon boundaries with actin serving as an endogenous control; data is normalized to +/+ mcub/mcub actin levels, and samples were run in triplicate with four biological replicates; data is shown as mean±s.d.;

FIG. 2D is a graph showing results from a qPCR using a custom TaqMan gene expression assay to detect transcripts with an exon 1-exon 7 boundary; samples were run in triplicate with three biological replicates (each bar represents an individual mouse); data is shown as mean±s.d.;

FIG. 2E is an image of an anti-Flag immunoblot of HEK293 cells transiently expressing Flag-tagged HuWt (human full-length RHBDF2 cDNA) and HuCub (human version of cub cDNA) with actin serving as a loading control;

FIG. 2F shows representative images of Flag-tagged HuWt and HuCub-expressing B6 primary MEFs stained using a Flag-specific antibody, and DAPI was used to counterstain the nucleus; arrows represent cytoplasmic expression of both full-length and mutant proteins;

FIG. 3A is an image showing the results from a PCR for wild-type product in KOMP Rhbdf2 knockout mice with an expected product length of 2181 bp; L represents the 1 kb DNA ladder obtained from New England Biolabs, and ntc represents the no template control;

FIG. 3B shows images for reporter gene analysis of Rhbdf2 expression; the whole-mount X-gal stained E18.5 Rhbdf2^{-/-} embryo shows strong expression of β-gal in the epidermis (arrowhead) (original magnification, ×2.5), and the X-gal stained two-week-old female Rhbdf2^{-/-} skin shows β-gal positivity in the inner and outer sheath layers of hair follicles (arrow) (original magnification, ×5); no staining was observed in the hair shaft; scale bar, 1 mm;

FIG. 3C is a graph showing that mice with a null mutation of Rhbdf2 lack a regenerative phenotype as demonstrated by significantly delayed wound closure in the ears of six to eight-week-old female Rhbdf2^{-/-} mice compared with those of Rhbdf2^{cub/cub} mice;

FIG. 3D shows images demonstrating that mice with a null mutation of Rhbdf2 have normal skin and hair morphology; H&E stained sections of adult skin from Rhbdf2^{cub/cub} and Rhbdf2^{-/-} mice taken at indicated times after wounding in which the adult Rhbdf2^{cub/cub} skin displays a thin hypodermal fat layer (F), abnormal hair follicles (H), thick epidermis (E), enlarged sebaceous glands (SG), dense interlacing bundles of collagen fibers (C), and no full differentiation of hair follicles and hair bulb (HF); the adult Rhbdf2^{-/-} skin shows normal epidermis and hair follicles; scale bar, 100 μm; original magnification, ×10 and ×40;

FIG. 3E is an image showing that Rhbdf2^{-/-} mice develop a normal hair coat (arrowhead), whereas compound mutant Rhbdf2^{-/cub} mice exhibit a sparse hair coat (arrow);

FIG. 4A is a diagram showing that Mcub is a T-to-G point mutation that disrupts the normal donor splice site (exon 1) in the Areg gene, causing the use of an alternative downstream splice site;

FIG. 4B is a graph showing the healing of ear holes in 6-8-week-old female Rhbdf2^{+/+} Areg^{+/+}, Rhbdf2^{cub/cub} Areg^{+/+}, and Rhbdf2^{cub/cub} Areg^{Mcub/Mcub} mice (n=3 mice per group) over a period of 28 days; data is shown as mean±s.d.;

FIG. 4C shows images of hematoxylin/eosin-stained sections showing post-excision healing of ear holes of Rhbdf2^{cub/cub} Areg^{Mcub/Mcub} mice in which the dotted line indicates the site of excision; original magnification, ×10; E represents epidermis and M represents proliferation; compare with FIG. 1F;

FIG. 4D is a graph showing serum AREG levels in age-matched Rhbdf2^{+/+} Areg^{+/+}, Rhbdf2^{cub/cub} Areg^{+/+}, and Rhbdf2^{cub/cub} Areg^{Mcub/Mcub} female mice, in which AREG was not detected (n.d.) in the serum of Rhbdf2^{cub/cub} Areg^{Mcub/Mcub} mice; data is shown as mean±s.d. of three independent experiments; ***p<0.001;

FIG. 4E is a graph of an ELISA quantitation of AREG levels in the supernatants of cultured mouse epidermal keratinocytes (MEKs) isolated from Rhbdf2^{+/+} Areg^{+/+}, Rhbdf2^{cub/cub} Areg^{+/+}, and Rhbdf2^{cub/cub} Areg^{Mcub/Mcub} mice, in which AREG was not detected (n.d.) in MEKs from Rhbdf2^{cub/cub} Areg^{Mcub/Mcub} mice; data is shown as mean±s.d. of three independent experiments ***p<0.001;

FIG. 4F is a graph showing results obtained from a qPCR of EGFR ligands: cDNA was extracted from skin tissues of Rhbdf2^{cub/cub} Areg^{+/+} and Rhbdf2^{+/+} Areg^{+/+} using TaqMan gene-expression assays where actin served as an endogenous control; data is normalized to Rhbdf2^{+/+} Areg^{+/+} actin levels, data is shown as mean±s.d. of three independent experiments; *p<0.05;

FIG. 5A is a diagram showing the schematic representation of the full-length human RHBDF2 (HuWt) gene and a human construct mimicking the mouse Rhbdf2^{cub} gene (HuCub);

FIG. 5B is a graph showing an ELISA quantitation of cleaved AREG after co-expression of HuWt or HuCub with the AREG gene in 293T cells; at 24 h post-transfection, cells were either incubated with DMSO or 10 μM marimastat (MM) for 24 h and AREG levels were analyzed in the conditioned medium with the transfections performed in duplicate, and the conditioned medium diluted five-fold;

FIG. 5C is a graph showing the quantitation of serum TNFα levels by ELISA 3 h after LPS injection of 8-12-week-old female mice of the indicated genotypes, in which TNFα was not detected (n.d.) in the serum of Rhbdf2^{+/+} mice with no LPS injection; *p<0.05;

FIG. 5D is a graph showing the ELISA quantitation of cleaved AREG/HB-EGF/EGF after co-expression of the human RHBDL2 (HuRHBDL2) gene with AREG or HB-EGF or EGF genes; at 24 h post-transfection, cells were incubated with 10 μM marimastat (MM) for 24 h and AREG/HB-EGF/EGF levels were analyzed in the conditioned medium, where HB-EGF and EGF conditioned medium were undiluted, and AREG medium was diluted five-fold; data represent mean±s.d. of three independent experiments; ***p<0.001;

FIG. 5E is a graph showing an ELISA quantitation of cleaved HB-EGF after co-expression of HuWt or HuCub with the HB-EGF gene in 293T cells; at 24 h post-transfection, cells were incubated with 10 μM marimastat (MM) for 24 h and HB-EGF levels were analyzed in the conditioned medium; ***p<0.001;

FIG. 5F is a graph showing an ELISA quantitation of cleaved EGF after co-expression of HuWt or HuCub with the EGF gene in 293T cells; at 24 h post-transfection, cells were either incubated with DMSO or 10 μM marimastat (MM) for 24 h and EGF levels were analyzed in the conditioned medium; **p<0.01;

FIG. 5G is a graph showing an ELISA quantitation of cleaved AREG after co-expression of HuCub or HuCub without the peptidase domain (HuCubΔPD) with the AREG gene in 293T cells; at 24 h post-transfection, cells were incubated with 10 μM marimastat (MM) for 24 h and AREG levels were analyzed in the conditioned medium; ***p<0.001;

FIG. 5H is a graph showing an ELISA quantitation of cleaved AREG after co-expression of HuCub or HuCub with individual alanine mutations with the AREG gene in 293T cells; at 24 h post-transfection, cells were incubated with 10 μM marimastat (MM) for 24 h and AREG levels were analyzed in the conditioned medium: ***p<0.001;

FIG. 5I is a graph showing an ELISA quantitation of cleaved EGF after co-expression of HuWt or HuCub or HuCub with individual alanine mutations with the EGF gene in 293T cells; at 24 h post-transfection, cells were incubated with 10 μM marimastat (MM) for 24 h and EGF levels were analyzed in the conditioned medium: **p<0.001;

FIG. 5J is a diagram showing membrane topology of Rhbdf2^{cub}, in which the amino acids shown are critical for regulation of EGFR ligand production by Rhbdf2^{cub};

FIG. 6A is a graph showing the quantitation of cleaved AREG by ELISA from conditioned medium of HEK293 cells transfected with AREG and either the human RHBDF2 p.I186T mutant, HuWt or HuCub, in the presence of 10 μM marimastat (MM); **p<0.01;

FIG. 6B is an image showing a Western blot of HEK293 cells co-transfected with AREG and either HuCub, HuWt or p.I186T, incubated with DMSO or MM, and immunoblotted for HA-AREG showing that co-expression of AREG and either HuCub or p.I186T significantly reduces the intracellular levels of pro-AREG compared with that of AREG/HuWt or AREG alone, even in the presence of 10 μM MM;

FIG. 6C is a graph showing AREG levels in cells expressing various RHBDF2 p.I186T point mutants and co-transfected with the AREG gene or empty vector; each residue was mutated to alanine; the cells were incubated with 10 μM MM for 24 h and assayed for AREG levels in conditioned medium;

FIG. 6D is an image of an X-gal-stained 2-week-old female Rhbdf2^{-/-} intestine that reveals reporter gene β-gal expression in the mid and upper villous regions; original magnification, ×2.5;

FIG. 6E is a graph showing Kaplan-Meier survival curves of Apc^{Min/+} Rhbdf2^{+/+} (n=28) and Apc^{Min/+} Rhbdf2^{+/cub} (n=23) mice in which the median survival of Apc^{Min/+} Rhbdf2^{+/+} mice was 172 days, compared with 135 days for Apc^{Min/+} Rhbdf2^{+/cub} mice;

FIG. 6F shows images of hematoxylin-eosin-stained sections of intestinal tissue from mice of the indicated genotypes at 3 months of age; original magnification, ×2.5;

FIG. 6G is a graph showing the size of polyps per mouse of the indicated genotypes of mice at 3 months of age; the mean tumor size was 1.8 mm² and 3.7 mm² for Apc^{Min/+} Rhbdf2^{+/+} and Apc^{Min/+} Rhbdf2^{+/cub} mice, respectively;

FIG. 6H is a graph showing the number of polyps per mouse of the indicated genotypes of mice at 3 months of age; the mean number of polyps was 12 and 21 for Apcs^{Min/+} Rhbdf2^{+/+} and Apc^{Min/+} Rhbdf2^{+/cub} mice, respectively;

FIG. 7A is an image of co-immunoprecipitation of human iRhom2-AREG complex in which lysates from COS7 cells were co-transfected with Flag-tagged HuWt or Flag-tagged HuCub or Flag-tagged p.I186T and HA-tagged AREG were immunoprecipitated with anti-Flag magnetic beads and probed with anti-Flag and anti-HA antibodies;

FIG. 7B shows flow cytometry results from 293T or COS7 (FIG. 14) cells transfected with the indicated Flag-tagged genes and immunolabeled using a Flag-specific PE-labeled antibody wherein the cells were immunolabeled 48 h post transfection;

FIG. 7C is a graph showing the quantification of the data obtained in FIG. 7B;

FIG. 7D shows results of tests in which transiently transfected COS7 cells were subjected to a chase with 150 μg/ml of the protein-synthesis inhibitor cyclohexamide for the indicated times and evaluated for protein expression using Flag-specific PE labeled antibody; data is representative of one of a total of three experiments;

FIG. 7E is a graph showing the quantification of the data obtained in FIG. 7D;

FIG. 7F is a graph showing results of tests in which transiently transfected COS7 cells were pre-incubated for 4 h with 10 μM MG-132, a cell permeable protease inhibitor, followed by a chase with 150 μg/ml cyclohexamide in presence of MG-132 for the indicated times; protein expression was determined as described in FIG. 7D; ***p<0.001;

FIG. 8 is a diagram showing that wild-type iRhom2 is a short-lived protein and loss of its N terminus or mutations in its N-terminal domain, including those that underlie epithelial cancers, increase its protein stability which in turn augments the secretion of selective EGF family ligands, including AREG; inhibition of ADAM17 has no effect on AREG secretion, whereas loss of amino acids H366, Q426, C432, and H475 in the peptidase domain of iRhom2 abrogates AREG secretion and conversely, enhanced secretion of AREG leads to hyperactivation of EGFR signaling and thereby increased cell proliferation and migration;

FIG. 9A is an image showing cub/cub mcub/mcub (arrow) and cub/cub Mcub/mcub (arrow head) mice;

FIG. 9B is a graph showing that wound healing in +/cub mcub/mcub appears intermediate between cub/cub mcub/mcub and +/+ mice; quantification of ear hole closure rates in 6 to 40-week-old female +/+ mcub/mcub, +/cub mcub/mcub and cub/cub mcub/mcub mice (n=3 per group) at 0, 7, 14, and 21 days post-wounding (2-mm through-and-through holes); data is shown as mean±s.d;

FIG. 10A shows images demonstrating in vitro expression of Flag-tagged full-length (HuWt) and cub (HuCub) Rhbdf2 clones in B6 primary MEFs followed by immunochemical assessment of protein expression with Flag-specific antibody and DAPI counterstaining for nucleus and showing that both full-length and mutant proteins co-localize with the ER marker calnexin;

FIG. 10B shows images demonstrating in vitro expression of Flag-tagged full-length (HuWt) and cub (HuCub) Rhbdf2 clones in B6 primary MEFs followed by immunochemical assessment of protein expression with Flag-specific antibody and DAPI counterstaining for nucleus and showing that both full-length and mutant proteins do not co-localize with the Golgi marker giantin;

FIG. 11 is a diagram showing that a T to G point mutation disrupts the original splice site (exon 1) leading to an alternative splice site that adds 22 additional nucleotides to the Areg transcript and 16 incorrect amino acids to the protein, introducing a premature stop codon;

FIG. 12A is an image of immunoblot analysis of cub/cub Mcub/Mcub or cub/cub mcub/mcub MEFs for the markers indicated in which cell lysates were run in duplicate and actin served as a loading control;

FIG. 12B is a graph showing a quantitative RT-PCR determination of Areg expression in scramble shRNA or mouse Areg shRNA lentiviral infected Rhbdf2^{+/+} or Rhbdf2^{cub/cub} MEFs;

FIG. 12C is a graph showing a quantitation of Rhbdf2^{+/+} MEFs using a proliferation assay, described herein, after an infection with scramble shRNA or mouse Areg shRNA in which the cells were seeded at the numbers indicated on the X-axis and incubated for 24 h; the extent of fluorescence is proportional to the amount of total cellular DNA;

FIG. 12D is a graph showing a quantitation of Rhbdf2^{cub/cub} MEFs using a proliferation assay, described herein, after an infection with scramble shRNA or mouse Areg shRNA in which the cells were seeded at the numbers indicated on the X-axis and incubated for 24 h; the extent of fluorescence is proportional to the amount of total cellular DNA;

FIG. 13A is a graph showing an ELISA quantitation of secreted AREG from a conditioned medium of HEK 293T cells transfected either with the mouse Rhbdf1 or N-terminus truncated mouse Rhbdf1 (ΔN-iRhom1) or ΔN-iRhom1 mutants together with the AREG gene in the presence of 10 μM marimastat (MM); ***p<0.001;

FIG. 13B is a graph showing an ELISA quantitation of cleaved EGF from a conditioned medium of HEK 293T cells transfected either with the mouse Rhbdf1 or N-terminus truncated mouse Rhbdf1 (ΔN-iRhom1) together with the EGF gene in the presence of 10 μM marimastat (MM); ***p<0.001;

FIG. 13C is an image of the alignment of the amino acids of the peptidase domain sequences of human iRhom2 and RHBDL2, and human and mouse iRhoms;

FIG. 13D is a graph showing an ELISA quantitation of secreted AREG after co-expression of HuCub or HuCub with individual alanine mutations with the AREG gene in 293T cells; at 24 h post-transfection, the cells were incubated with 10 μM marimastat (MM) for 24 h and AREG levels were analyzed in the conditioned medium; data represent mean±s.d. of three independent experiments;

FIG. 13E is a diagram showing the sequence alignment for the amino acids of the peptidase domains of iRHOM1 and iRHOM2, the active rhomboid RHBDL2, the C. elegans inactive rhomboids ROM3 and ROM4, and Drosophila iRhom wherein the asterisks represent conserved amino acids critical for regulation of EGFR ligands by iRHOM2; and

FIG. 14 shows images of flow cytometry results from COS7 cells transfected with the indicated Flag-tagged genes and immunolabeled using a Flag-specific PE-labeled antibody in which the cells were immunolabeled 48 h post transfection.

### DETAILED DESCRIPTION OF THE INVENTION

Scientific and technical terms used herein are intended to have the meanings commonly understood by those of ordinary skill in the art. Such terms are found defined and used in context in various standard references illustratively including J. Sambrook and D. W. Russell, Molecular Cloning: A Laboratory Manual, Cold Spring Harbor Laboratory Press; 3rd Ed., 2001; F. M. Ausubel, Ed., Short Protocols in Molecular Biology, Current Protocols; 5th Ed., 2002; B. Alberts et al., Molecular Biology of the Cell, 4th Ed., Garland, 2002; D. L. Nelson and M. M. Cox, Lehninger Principles of Biochemistry, 4th Ed., W.H. Freeman & Company, 2004; Engelke, D. R., RNA Interference (RNAi): Nuts and Bolts of RNAi Technology, DNA Press LLC, Eagleville, Pa., 2003; Herdewijn, P. (Ed.), Oligonucleotide Synthesis: Methods and Applications, Methods in Molecular Biology, Humana Press, 2004; A. Nagy, M. Gertsenstein, K. Vintersten, R. Behringer, Manipulating the Mouse Embryo: A Laboratory Manual, 3rd edition, Cold Spring Harbor Laboratory Press; Dec. 15, 2002, ISBN-10: 0879695919; Kursad Turksen (Ed.), Embryonic stem cells: methods and protocols in Methods Mol Biol. 2002; 185, Humana Press; Current Protocols in Stem Cell Biology, ISBN: 9780470151808; Chu, E. and Devita, V. T., Eds., Physicians' Cancer Chemotherapy Drug Manual, Jones & Bartlett Publishers, 2005; J. M. Kirkwood et al., Eds., Current Cancer Therapeutics, 4th Ed., Current Medicine Group, 2001; Remington: The Science and Practice of Pharmacy, Lippincott Williams & Wilkins, 21st Ed., 2005; L. V. Allen, Jr. et al., Ansel's Pharmaceutical Dosage Forms and Drug Delivery Systems, 8th Ed., Philadelphia, Pa.: Lippincott, Williams & Wilkins, 2004; and L. Brunton et al., Goodman & Gilman's The Pharmacological Basis of Therapeutics, McGraw-Hill Professional, 12th Ed., 2011.

The singular terms “a,” “an,” and “the” are not intended to be limiting and include plural referents unless explicitly stated otherwise or the context clearly indicates otherwise.

Inactive rhomboids (iRhoms) are highly conserved intramembrane proteins that were previously thought to be protelytically inactive. iRhoms are characterized by a long cytosolic N-terminal domain, a conserved cysteine-rich inactive rhomboid homology domain (IRHD), a dormant proteolytic site lacking an active-site serine residue within the peptidase domain.

iRhoms participate in a diverse range of functions in a variety of species, including regulation of epidermal growth factor receptor (EGFR) signaling in Drosophila melanogaster, survival of human squamous epithelial cancer cells, misfolded protein clearance from endoplasmic reticulum membranes in mammalian cell lines, induction of migration in primary mouse keratinocytes, secretion of soluble TNFα in mice, and regulation of substrate selectivity of stimulated ADAM17-mediated metalloprotease shedding in mouse embryonic fibroblasts. EGF-like ligands may act as substrates for iRhom family members.

The Rhbdf2 gene encodes an inactive rhomboid protease iRhom2, one of a family of enzymes containing a long cytosolic N-terminus and a dormant peptidase domain of previously unknown function. The present invention provides that iRhom2 may function in epithelial regeneration and cancer growth through constitutive activation of EGFR signaling.

Aspects of the present invention are based, at least in part, on the discovery that Rhbdf2 mutations increase iRhom2 protein stability and drive EGFR hyperactivation through enhanced secretion of amphiregulin.

Aspects of the present invention are based, at least in part, on the discovery that iRhom2 is a short-lived protein whose stability can be increased by select mutations in the N-terminal domain. In turn, these stable variants function to augment the secretion of EGF family ligands, including amphiregulin, independently of metalloprotease ADAM17 activity. In vivo, N-terminal iRhom2 mutations induce accelerated wound healing as well as accelerated tumorigenesis, but do not drive spontaneous tumor development. Therefore, the present invention is also based, at least in part, on the physiological prominence of iRhom2 in controlling EGFR signaling events involved in wound healing and neoplastic growth, and the function of key iRhom2 domains.

Epidermal growth factor receptor (EGFR) signal transduction plays a major role in growth, proliferation and differentiation of mammalian cells. Canonical EGFR ligands, including EGF, AREG, and HB-EGF, exist as pro-proteins expressed at the cell surface that, in order to bind EGFRs, must be shed into the extracellular compartment. Different classes of proteases cleave membrane-tethered EGFR pro-ligands to regulate a broad range of biological activities during various stages of development.

In one aspect, the present invention is based, at least in part, on the discovery that a spontaneous deletion within the Rhbdf2 gene in mice underlies the curly bare (cub) mutation, in which loss of the cytosolic N-terminal domain of iRhom2 causes subsequent effects on hair-follicle development, wound healing and tumorigenesis. Furthermore, the modifier of cub (Mcub) cures or corrects balding and/or promotes hair growth (Example 1). In another aspect, the present invention is based, at least in part, on the discovery that iRhom2 is a short-lived protein but that gain-of-function mutations in the N-terminus (tylosis) or loss of the N-terminus (cub mutation) increase mutant protein stability leading to metalloprotease-independent secretion of the EGFR ligand amphiregulin (AREG). A genetic modifier of the cub phenotype (Mcub), was used to demonstrate that AREG is a physiological target of iRhom2. In another aspect, the present invention is based, at least in part, on the identification of key amino acids in the peptidase domain of iRhom2 that are necessary for AREG secretion, indicating that the peptidase domain of this pseudoenzyme might be functional despite lacking a serine residue in the putative active site. This invention therefore provides the function of key iRhom domains and establishes a framework for understanding the relationship between iRhoms, EGFR signaling and the biological processes involved in wound healing and tumorigenesis.

iRhom Family Members

iRhom1 and iRhom2

The rhomboid proteases are a family of enzymes that exist in almost all species. Rhomboids are intramembrane serine proteases and proteolytic cleavage by Rhomboid proteases is important for cellular regulation. The active site of intramembrane proteases is buried in the lipid bilayer of cell membranes, and they cleave other transmembrane proteins within their transmembrane domains.

iRhoms or “inactive Rhomboids” are a subfamily of the rhomboid proteases that prior to the present invention were thought to lack protelytic activity. iRhoms are highly conserved intramembrane proteins that are characterized by a long cytosolic N-terminal domain, a conserved cysteine-rich inactive rhomboid homology domain (IRHD), a dormant proteolytic site lacking an active-site serine residue within the peptidase domain, iRhoms family members that are suitable for use in the present invention include iRhom1 (RHBDF1) and iRhom2 (RHBDF2). iRhom1 and/or iRhom2 for use in the present invention can be eukaryotic, prokaryotic, or mammalian. Additionally, iRhom1 and/or iRhom2 for use in the present invention can be from human, primate, rodent (e.g., mouse, rat, guinea pig), bovine, equine, donkey, rabbit, goat, sheep, dog, chicken, pig, drosophila, C. elegans, or E. coli.

Wild-type iRhoms are exemplified herein as mouse iRhom2 (SEQ ID No:1); mouse iRhom1 (SEQ ID No:3); human iRhom2 (SEQ ID No:5); and human iRhom1 (SEQ ID No:7). Isolated mutated sequences of iRhoms are exemplified herein as SEQ ID NO:2; SEQ ID NO:4; SEQ ID NO:6; SEQ ID NO:8.

One of skill in the art will readily recognize a nucleic acid sequence encoding the iRhom polypeptides shown herein as SEQ ID NOs: 1-8. It is appreciated that due to the degenerate nature of the genetic code, more than one nucleic acid sequence encodes a particular iRhom polypeptide or variant, and that such nucleic acid sequences may be expressed to produce the desired iRhom.

iRhom1 and iRhom2 variants generally have a sequence identity to the sequence of wild-type iRhom1 and iRhom2 as set forth in the present application of at least 50%, such as at least 60%, at least 70%, at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99%.

In one aspect, the number of alterations, e.g., substitutions, insertions, or deletions, in the iRhom variants of the present invention is 1-20, e.g., 1-10 and 1-5, such as 1, 2, 3, 4, 5, 6, 7, 8, 9 or 10 alterations compared to the corresponding wild-type iRhom1 or iRhom2.

Mechanism of Action

iRhoms participate in a diverse range of functions in a variety of species, including regulation of epidermal growth factor receptor (EGFR) signaling in Drosophila melanogaster, survival of human squamous epithelial cancer cells, misfolded protein clearance from endoplasmic reticulum membranes in mammalian cell lines, induction of migration in primary mouse keratinocytes, secretion of soluble TNFα in mice, and regulation of substrate selectivity of stimulated ADAM17-mediated metalloprotease shedding in mouse embryonic fibroblasts.

In one embodiment, the present invention demonstrates that a spontaneous deletion within the Rhbdf2 gene in mice underlies the curly bare (cub) mutation, in which loss of the cytosolic N-terminal domain of iRhom2 causes subsequent effects on hair-follicle development, wound healing and tumorigenesis. In another embodiment, the present invention demonstrates that iRhom2 is a short-lived protein but that gain-of-function mutations in the N-terminus or loss of the N-terminus increase mutant protein stability leading to metalloprotease-independent secretion of the EGFR ligand amphiregulin (AREG). Using a genetic modifier of the cub phenotype (Mcub), the present invention demonstrates that AREG is a physiological target of iRhom2. In yet another embodiment, key amino acids (H, C, Q, H) in the peptidase domain of iRhom2 that are necessary for AREG secretion were identified, indicating that the peptidase domain of this pseudoenzyme might be functional despite lacking a serine residue in the putative active site.

Mutant Forms of iRhom Family Members

The present invention also relates to mutant forms of iRhom family members. In one embodiment the cytosolic N-terminus of iRhom1 or iRhom2 is deleted. The deletion of the cytosolic N-terminus activates the dormant proteolytic site within the peptidase domain of the iRhom polypeptide. In one embodiment, the cytosolic terminus of iRhom1 or iRhom2 is cleaved such that the transmembrane domain is proteolytically active. In one embodiment, mouse iRhom1 is cleaved between residues 1 and 272 and/or human iRhom1 is cleaved between residues 1 and 316 such that the transmembrane domain is proteolytically active. In another embodiment, mouse iRhom2 is cleaved between residues 1 and 268 and/or human iRhom2 is cleaved between residues 1 and 298 such that the transmembrane domain is proteolytically active.

The iRhom polypeptides may also comprise mutations in the proteolytic site of the transmembrane domain. In one embodiment, a mouse iRhom2 polypeptide comprises one or more mutations of key amino acids in the proteolytic site (peptidase domain) of the transmembrane domain selected from the group consisting of Histidine 635 on TM helix 2, Glutamine 695 on TM helix 4, Cysteine 701 on transmembrane helix 4 and Histidine 744 such that the transmembrane domain is proteolytically active. In another embodiment, a human iRhom2 polypeptide comprises one or more mutations in the proteolytic site of the transmembrane domain selected from the group consisting of Histidine 664 on TM helix 2, Glutamine 724 on TM helix 4, Cysteine 730 on transmembrane helix 4 and Histidine 773 on TM helix 6 such that the transmembrane domain is proteolytically active.

Screening Assays

The present invention provides methods for identifying a compound that alters the activity of an iRhom polypeptide, e.g., iRhom1 or iRhom2. The iRhom polypeptide may be a mutant iRhom polypeptide. For example, the iRhom polypeptide may comprise a deletion in its N-terminal cytoplasmic domain. The iRhom polypeptide may comprise a mutation within the transmembrane peptidase domain. In addition, the iRhom polypeptide may comprise a deletion in its N-terminal cytoplasmic domain and a mutation within the transmembrane peptidase domain.

In one aspect, methods of the invention generally include identifying a compound that activates the proteolytic activity of an iRhom polypeptide on a substrate. In one embodiment, the method includes contacting a cell expressing an iRhom polypeptide with a test compound and determining an increase in stability of the iRhom polypeptide relative to an appropriate control, wherein an increase in stability of the iRhom polypeptide indicates that the compound activates the proteolytic activity of the iRhom polypeptide on a substrate. In one embodiment, the substrate may be an EGFR ligand or an EGF-like substrate.

In another aspect, methods of the invention generally include identifying a compound that inhibits the proteolytic activity of an iRhom polypeptide on a substrate. In one embodiment, the method includes contacting a cell expressing an iRhom polypeptide with a test compound and determining a decrease in secretion of a physiological target of the iRhom polypeptide relative to an appropriate control or a decrease in EGFR activity relative to an appropriate control, wherein a decrease in secretion of a physiological target of the iRhom polypeptide or a decrease in EGFR activity indicates that the compound inhibits the proteolytic activity of the iRhom polypeptide on a substrate. In one embodiment, the substrate may be an EGFR ligand or an EGF-like substrate.

It is believed that Epidermal growth factor receptor (EGFR) signal transduction plays a major role in growth, proliferation and differentiation of mammalian cells. The present invention provides that iRhoms are short-lived proteins, but dominant mutations increase their protein stability and stimulate secretion of specific EGF family ligand amphiregulin independently of metalloprotease activity. This invention demonstrates the significance of mammalian iRhoms in regulating an EGFR signaling event that promotes accelerated wound healing and triggers tumorigenesis. The invention also provides that iRhoms may regulate EGFR signaling in parallel with metalloproteases. Therefore, the invention provides a new strategy for using iRhoms as a novel therapeutic target in impaired wound healing and cancer.

Various aspects of the present invention relate to screening and assay methods and means, and substances identified thereby, for example, assays for compounds that activate or inhibit the proteolytic activity of an iRhom polypeptide on a substrate. The substrate may be an EGFR ligand or an EGF-like substrate.

Further assays are for a compound or substance that interacts with or binds an iRhom polypeptide and modulates i.e. increases, stimulates, reduces, inhibits or abolishes, its proteolytic activity.

Controls are well-known in the art and one of skill in the art would readily recognize an appropriate control and be able to determine an appropriate control for a method of the present invention with no more than routine experimentation.

According to aspects of a method for identifying a compound that modulates activity of an iRhom polypeptide according to aspects of the present invention which includes contacting a cell expressing an iRhom polypeptide with a test compound and determining the effect of the test compound on activity of the iRhom polypeptide, an appropriate control is determining the activity of the iRhom polypeptide when a cell expressing the iRhom polypeptide is not contacted with the test compound under the same or similar conditions.

An appropriate control may be a reference level of a variable such as activity of an iRhom polypeptide, level of stability of the iRhom polypeptide, level of secretion of a physiological target of the iRhom polypeptide, level of EGFR activity relative to an appropriate control and/or level of a soluble EGFR ligand, previously determined and stored in a print or electronic medium for recall and comparison to a determined effect of a test compound on activity of an iRhom polypeptide according to aspects of a method for identifying a compound that modulates activity of an iRhom polypeptide of the present invention.

An assay method for identifying a modulator of an iRhom polypeptide may include bringing into contact an iRhom polypeptide as described herein and a test compound, determining binding of the test compound to the iRhom polypeptide and determining the proteolytic activity of the iRhom polypeptide in the presence and absence of a test compound which binds the iRhom polypeptide. Proteolytic activity may be determined by determining the cleavage of a substrate as described below. The iRhom polypeptide may be isolated or comprised in a liposome or cell.

A method of screening for and/or obtaining a substance/compound which modulates activity of an iRhom polypeptide may include contacting one or more test substances or compounds with the iRhom polypeptide in a suitable reaction medium, determining the activity of the treated polypeptide and comparing that activity with the activity of the polypeptide in comparable reaction medium untreated with the test substance or substances. The iRhom polypeptide may be in the reaction medium in an isolated form or may be comprised in a liposome or cell.

A difference in activity between the treated and untreated iRhom polypeptides is indicative of a modulating effect of the relevant test substance or substances, for example, an inhibiting or enhancing effect.

Activity of an iRhom polypeptide may be determined by determining the production of proteolytically cleaved substrate. The iRhom polypeptide may, for example, act on a membrane-bound substrate to generate a soluble product which is detected.

According to another aspect of the present invention there is provided an assay method for identifying and/or obtaining a modulator of an iRhom polypeptide, which method comprises: (a) bringing into contact an iRhom polypeptide and a test compound in the presence of a polypeptide substrate; and (b) determining proteolytic cleavage of the polypeptide substrate.

Cleavage of the substrate may be determined in the presence and absence of test compound. A difference in cleavage in the presence of the test compound relative to the absence of test compound may be indicative of the test compound being a modulator of iRhom protease activity.

Any polypeptide substrate which is proteolytically cleaved by an iRhom polypeptide may be used in an assay method as described herein. Such substrates are readily identified using standard techniques. The polypeptide substrate may be an EGFR ligand, such as an AREG or HB-EGF.

A suitable substrate may comprise a detectable label such as green fluorescent protein (GFP), luciferase or alkaline phosphatase. This allows convenient detection of the soluble cleaved product and is particularly useful in automated assays.

EGFR ligands suitable for use in the present assays are well characterized in the art and may have a structure comprising one or more Epidermal Growth Factor (EGF) domains and a single trans-membrane domain.

Preferably, suitable EGFR ligands have greater than 50% homology, greater than 60% homology, greater than 70% homology, greater than 80% homology greater than 90% homology or greater than 95% homology to a vertebrate EGFR ligand.

A chimeric ligand may have improved properties in methods described herein, for example it may be cleaved more efficiently by an iRhom polypeptide, have improved secretion properties or be more readily detected.

Another aspect of the present invention provides a chimeric EGFR ligand comprising sequence from two or more EGFR ligands, for example a chimeric ligand may comprise the transmembrane domain of a first EGFR ligand and the intracellular and extracellular domains of a second EGFR ligand. A chimeric substrate may further comprise a detectable label, such as luciferase, GFP or alkaline phosphatase.

Assay methods or other methods for obtaining or identifying modulators of iRhom activity according to the present invention may be in vivo cell-based assays, or in vitro non-cell-based assays.

Methods may be performed in the presence of Batimastat to inhibit the non-Rhomboid or non-iRhom dependent shedding of substrate and thereby decrease background.

In in vitro assays, the iRhom polypeptide may be isolated or contained in a liposome. Liposome based assays may be carried out using methods well-known in the art.

Suitable cell types for in vitro assays include mammalian cells such as mouse embryonic fibroblasts, keratinocytes, HEK293, CHO, HeLa and COS cells.

It is not necessary to use the entire full length proteins for in vitro or in vivo assays of the invention. Polypeptide fragments as described herein which retain the activity of the full length protein may be generated and used in any suitable way known to those of skill in the art. Suitable ways of generating fragments include, but are not limited to, recombinant expression of a fragment from encoding DNA. Such fragments may be generated by taking encoding DNA, identifying suitable restriction enzyme recognition sites either side of the portion to be expressed, and cutting out said portion from the DNA. The portion may then be operably linked to a suitable promoter in a standard commercially available expression system. Another recombinant approach is to amplify the relevant portion of the DNA with suitable PCR primers. Small fragments (e.g. up to about 20 or 30 amino acids) may also be generated using peptide synthesis methods which are well known in the art.

The precise format of the assay of the invention may be varied by those of skill in the art using routine skill and knowledge. For example, interaction between the polypeptides may be studied in vitro by labeling one with a detectable label and bringing it into contact with the other which has been immobilized on a solid support. Suitable detectable labels include 35S-methionine which may be incorporated into recombinantly produced peptides and polypeptides. Recombinantly produced peptides and polypeptides may also be expressed as a fusion protein containing an epitope that can be labeled with an antibody.

Fusion proteins may be generated that incorporate six histidine residues at either the N-terminus or C-terminus of the recombinant protein. Such a histidine tag may be used for purification of the protein by using commercially available columns which contain a metal ion, either nickel or cobalt. These tags also serve for detecting the protein using commercially available monoclonal antibodies directed against the six histidine residues.

Assays according to the present invention may take the form of in vitro assays. In vitro assays may be performed in a cell line such as a yeast strain, insect cell line or mammalian cell line in which the relevant polypeptides or peptides are expressed from one or more vectors introduced into the cell.

In assay and other methods according to such embodiments, an iRhom polypeptide may be contacted with the test compound in the presence of a substrate, such as an EGFR ligand. In such methods, the iRhom polypeptide and substrate may be present in a cell. This may be achieved, for example, by expressing the polypeptides from one or more expression vectors which have been introduced into the cell by transformation.

An assay method for identifying and/or obtaining a modulator of iRhom proteolytic activity may therefore include: (a) bringing into contact an iRhom polypeptide and a test compound in the presence of an EGFR ligand polypeptide; and (b) determining cleavage of the EGFR ligand.

Cleavage may be determined in the presence and absence of test compound. A difference in cleavage in the presence, relative to the absence of test compound is indicative of the compound being a modulator i.e. an enhancer or inhibitor of iRhom activity.

A nucleic acid encoding an iRhom polypeptide and/or polypeptide substrate as described above may be provided as part of a replicable vector, particularly any expression vector from which the encoded polypeptide can be expressed under appropriate conditions, and a host cell containing any such vector or nucleic acid. An expression vector in this context is a nucleic acid molecule including nucleic acid encoding a polypeptide of interest and appropriate regulatory sequences for expression of the polypeptide, in an in vitro expression system, e.g. reticulocyte lysate, or in vivo, e.g. in eukaryotic cells such as HEK, COS or CHO cells or in prokaryotic cells such as E. coli. This is discussed further below.

Combinatorial library technology provides an efficient way of testing a potentially vast number of different substances for ability to modulate activity of a polypeptide. Prior to or as well as being screened for modulation of activity, test substances may be screened for ability to interact with the polypeptide, e.g. in a yeast two-hybrid system (which requires that both the polypeptide and the test substance can be expressed in yeast from encoding nucleic acid). This may be used as a coarse screen prior to testing a substance for actual ability to modulate activity of the polypeptide.

The amount of test substance or compound which may be added to an assay of the invention will normally be determined by trial and error depending upon the type of compound used. Typically, from about 0.01 to 100 nM concentrations of putative inhibitor or activator compound may be used, for example from 0.1 to 10 nM. When cell-based assays are employed, the test substance or compound is desirably membrane permeable in order to access the iRhom polypeptide.

Test compounds may be natural or synthetic chemical compounds used in drug screening programs. Extracts of plants which contain several characterized or uncharacterized components may also be used. A further class of putative inhibitor or activator compounds can be derived from the iRhom polypeptide and/or a ligand which binds. Membrane permeable peptide fragments of from 5 to 40 amino acids, for example, from 6 to 10 amino acids may be tested for their ability to disrupt such interaction or activity.

In one embodiment, compounds for modulating (e.g., increase or decrease) the proteolytic activity of an iRhom polypeptide may be small molecule compounds. In another embodiment, the compound is cell-permeable. For example, the compound may be an ubiquitin protease inhibitor. In another embodiment, the compound is attached to a cell-penetrating peptide. The cell penetrating peptide may comprise a lysine or arginine rich sequence. For example, the cell penetrating peptide may be tat.

Other test compounds may be based on modeling the 3-dimensional structure of a polypeptide or peptide fragment and using rational drug design to provide potential inhibitor compounds with particular molecular shape, size and charge characteristics.

Following identification of a substance which modulates or affects polypeptide activity, the substance may be investigated further. Furthermore, it may be manufactured and/or used in preparation, i.e. manufacture or formulation, of a composition such as a medicament, pharmaceutical composition or drug. These may be administered to individuals.

Another aspect of the present invention provides the use of an iRhom polypeptide as described herein in a method for obtaining or identifying a modulator, for example an inhibitor, of iRhom proteolytic activity. Also provided are methods and uses of an iRhom polypeptide in the proteolytic cleavage of the transmembrane domain of a polypeptide substrate.

Modulators, in particular inhibitors of iRhom activity may be useful in the treatment of cancer, for example, esophageal, lung, brain, colon, kidney, prostate, skin, liver, pancreatic, stomach, uterine, ovarian, lymph glands or bladder cancer.

Modulators, in particular activators of iRhom activity may be useful in the acceleration of impaired wound healing in tissues such as skin, liver, heart, muscle or kidney.

Modulators of iRhom activity may also be useful in the treatment of inflammatory disorders or diseases, such as rheumatoid arthritis and autoimmune disease.

Thus, the present invention extends in various aspects not only to a substance identified as a modulator of iRhom activity, in accordance with what is disclosed herein, but also a pharmaceutical composition, medicament, drug or other composition comprising such a substance, a method comprising administration of such a composition to a patient, e.g. for treatment (which may include preventative treatment) of a pathogenic infection or a condition associated with aberrant ErbB or EGF receptor activity, such as cancer, coronary atherosclerosis, psoriasis, wound healing, hair growth, curing and/or correcting balding, use of such a substance in manufacture of a composition for administration, e.g. for treatment of a pathogenic infection or a condition associated with aberrant ErbB or EGF receptor activity, such as cancer, coronary atherosclerosis, psoriasis, wound healing, hair growth, curing and/or correcting balding, and a method of making a pharmaceutical composition comprising admixing such a substance with a pharmaceutically acceptable excipient, vehicle or carrier, and optionally other ingredients.

A substance identified as a modulator of polypeptide or promoter function using an assay described herein may be peptide or non-peptide in nature. Non-peptide “small molecules” are often preferred for many in vivo pharmaceutical uses. Accordingly, a mimetic or mimic of the substance (particularly if a peptide) may be designed for pharmaceutical use.

The designing of mimetics to a known pharmaceutically active compound is a known approach to the development of pharmaceuticals based on a “lead” compound. This might be desirable where the active compound is difficult or expensive to synthesize or where it is unsuitable for a particular method of administration.

Mimetic design, synthesis and testing may be used to avoid randomly screening large number of molecules for a target property.

The essential catalytic residues of polypeptides of the iRhom family correspond to Histidine 635 on TM helix 2, Glutamine 695 on TM helix 4, Cysteine 701 on TM helix 4, and Histidine 744 on TM helix 6 of the mouse iRhom2 polypeptide. The essential catalytic residues of polypeptides in the iRhom family also correspond to Histidine 664 on TM helix 2, Glutamine 724 on TM helix 4, Cysteine 730 on TM helix 4, and Histidine 773 on TM helix 6 of the human iRhom2 polypeptide.

A compound, polypeptide, peptide or substance able to modulate activity of a polypeptide according to the present invention may be provided in a kit, e.g. sealed in a suitable container which protects its contents from the external environment. Such a kit may include instructions for use.

Whether it is a polypeptide, antibody, peptide, nucleic acid molecule, small molecule or other pharmaceutically useful compound according to the present invention that is to be given to an individual, administration is preferably in a “prophylactically effective amount” or a “therapeutically effective amount” (as the case may be, although prophylaxis may be considered therapy), this being sufficient to show benefit to the individual. The actual amount administered, and rate and time-course of administration, will depend on the nature and severity of what is being treated. Prescription of treatment, e.g. decisions on dosage etc, is within the responsibility of general practitioners and other medical doctors.

A composition may be administered alone or in combination with other treatments, either simultaneously or sequentially dependent upon the condition to be treated.

Compounds of the Invention

Compounds that Alter the Proteolytic Activity of iRhom Polypeptides

Compounds or test compounds of the invention may alter the proteolytic activity of iRhom polypeptides. In one embodiment, the compounds include activators or inhibitors of the proteolytic activity of iRhom polypeptides.

In one embodiment, the compound is a small molecule which binds an iRhom polypeptide. In another embodiment, the compound is a small molecule that inhibits the activity of an iRhom polypeptide. In yet another embodiment, the compound is a small molecule that activates the activity of an iRhom polypeptide. These small molecules may include, for example, peptides, peptidomimetics, organic compounds and the like. In one embodiment, the small molecule compound can translocate through plasma membranes and interact with the iRhom polypeptide. In some embodiment, the small molecule compound interacts with the cytoplasmic tail of the iRhom polypeptide. In other embodiments, the small molecule compound interacts with the proteolytic domain of the iRhom polypeptide.

In one embodiment, the compound is a ubiquitinase inhibitor.

In another embodiment, compounds that alter the proteolytic activity of an iRhom polypeptide may include, for example, a miRNA, a siRNA, a shRNA, a dsRNA or an antisense RNA directed to an iRhom DNA or mRNA, or a polynucleotide encoding the miRNA, siRNA, shRNA, dsRNA or antisense RNA, a vector comprising the polynucleotide. In yet another embodiment, compounds that alter the proteolytic activity of an iRhom polypeptide include, for example, antibodies, antibody fragments, a peptide or a polypeptide decoy. Additional compounds may be identified from combinatorial chemistry inhibitor libraries by screens, and then further optimized through chemical alterations.

In another embodiment, the compound further comprises a cell penetrating peptide. The cell penetrating peptide, in one aspect, comprises a HIV-TAT peptide.

Pharmaceutical Compositions

The compounds on the invention that alter the proteolytic activity of an iRhom polypeptide may be administered using a pharmaceutical composition. Suitable pharmaceutical compositions comprise any one of the compounds described herein (or a pharmaceutically acceptable salt or ester thereof), and optionally comprise a pharmaceutically acceptable carrier. In certain embodiments, these compositions optionally further comprise one or more additional therapeutic agents.

Acceptable “pharmaceutical carriers” are well known to those of skill in the art and can include, but not be limited to any of the standard pharmaceutical carriers, such as phosphate buffered saline, water and emulsions, such as oil/water emulsions and various types of wetting agents.

As used herein, the term “pharmaceutically acceptable salt” refers to those salts which are, within the scope of sound medical judgment, suitable for use in contact with the tissues of humans and lower animals without undue toxicity, irritation, allergic response and the like, and are commensurate with a reasonable benefit/risk ratio. Pharmaceutically acceptable salts of amines, carboxylic acids, and other types of compounds, are well known in the art. For example, S. M. Berge, et al. describe pharmaceutically acceptable salts in detail in J. Pharmaceutical Sciences, 66: 1-19 (1977), incorporated herein by reference. The salts can be prepared in situ during the final isolation and purification of the compounds of the invention, or separately by reacting a free base or free acid function with a suitable reagent, as described generally below.

For example, a free base function can be reacted with a suitable acid. Furthermore, where the compounds of the invention carry an acidic moiety, suitable pharmaceutically acceptable salts thereof may, include metal salts such as alkali metal salts, e.g. sodium or potassium salts; and alkaline earth metal salts, e.g. calcium or magnesium salts. Examples of pharmaceutically acceptable, nontoxic acid addition salts are salts of an amino group formed with inorganic acids such as hydrochloric acid, hydrobromic acid, phosphoric acid, sulfuric acid and perchloric acid or with organic acids such as acetic acid, oxalic acid, maleic acid, tartaric acid, citric acid, succinic acid or malonic acid or by using other methods used in the art such as ion exchange. Other pharmaceutically acceptable salts include adipate, alginate, ascorbate, aspartate, benzenesulfonate, benzoate, bisulfate, borate, butyrate, camphorate, camphorsulfonate, citrate, cyclopentanepropionate, digluconate, dodecylsulfate, ethanesulfonate, formate, fumarate, glucoheptonate, glycerophosphate, gluconate, hernisulfate, heptanoate, hexanoate, hydroiodide, 2-hydroxy-ethanesulfonate, lactobionate, lactate, laurate, lauryl sulfate, malate, maleate, malonate, methanesulfonate, 2-naphthalenesulfonate, nicotinate, nitrate, oleate, oxalate, palmitate, pamoate, pectinate, persulfate, 3-phenylpropionate, phosphate, picrate, pivalate, propionate, stearate, succinate, sulfate, tartrate, thiocyanate, p-toluenesulfonate, undecanoate, valerate salts, and the like. Representative alkali or alkaline earth metal salts include sodium, lithium, potassium, calcium, magnesium, and the like. Further pharmaceutically acceptable salts include, when appropriate, nontoxic ammonium, quaternary ammonium, and amine cations formed using counterions such as halide, hydroxide, carboxylate, sulfate, phosphate, nitrate, lower alkyl sulfonate and aryl sulfonate.

The term “pharmaceutically acceptable ester”, as used herein, refers to esters that hydrolyze in vivo and include those that break down readily in the human body to leave the parent compound or a salt thereof. Suitable ester groups include, for example, those derived from pharmaceutically acceptable aliphatic carboxylic acids, particularly alkanoic, alkenoic, cycloalkanoic and alkanedioic acids, in which each alkyl or alkenyl moiety advantageously has not more than 6 carbon atoms. Examples of particular esters include formates, acetates, propionates, butyrates, acrylates and ethylsuccinates.

As described above, the pharmaceutical compositions may additionally comprise a pharmaceutically acceptable carrier. Such a carrier includes any and all solvents, diluents, or other liquid vehicle, dispersion or suspension aids, surface active agents, isotonic agents, thickening or emulsifying agents, preservatives, solid binders, lubricants and the like, as suited to the particular dosage form desired. Remington's Pharmaceutical Sciences, Sixteenth Edition, E. W. Martin (Mack Publishing Co., Easton, Pa., 1980) discloses various carriers used in formulating pharmaceutical compositions and known techniques for the preparation thereof. Except insofar as any conventional carrier medium is incompatible with the compound, such as by producing any undesirable biological effect or otherwise interacting in a deleterious manner with any other component(s) of the pharmaceutical composition, its use is contemplated to be within the scope of this invention. Some examples of materials which can serve as pharmaceutically acceptable carriers include, but are not limited to, sugars such as lactose, glucose and sucrose; starches such as corn starch and potato starch; cellulose and its derivatives such as sodium carboxymethyl cellulose, ethyl cellulose and cellulose acetate; powdered tragacanth; malt; gelatine; talc; excipients such as cocoa butter and suppository waxes; oils such as peanut oil, cottonseed oil; safflower oil, sesame oil; olive oil; corn oil and soybean oil; glycols; such as propylene glycol; esters such as ethyl oleate and ethyl laurate; agar; buffering agents such as magnesium hydroxide and aluminum hydroxide; alginic acid; pyrogenfree water; isotonic saline; Ringer's solution; ethyl alcohol, and phosphate buffer solutions, as well as other non-toxic compatible lubricants such as sodium lauryl sulfate and magnesium stearate, as well as coloring agents, releasing agents, coating agents, sweetening, flavoring and perfuming agents, preservatives and antioxidants can also be present in the composition, according to the judgment of the formulator.

Compositions for use in the present invention may be formulated to have any concentration of the compound desired. In some embodiments, the composition is formulated such that it comprises at least a therapeutically effective amount of the compound that alters proteolytic activity of the iRhom polypeptide. A therapeutically effective amount is an amount sufficient to achieve the desired therapeutic effect, under the conditions of administration, such as an amount sufficient to treat a cancer, accelerate wound healing or treat an inflammatory condition. In some embodiments, the composition is formulated such that it comprises an amount that would not cause one or more unwanted side effects. In certain embodiments, compositions are formulated so that the compound is present at a concentration of between about 1 mg/mL and about 20 mg/mL; between about 1 mg/mL and about 15 mg/mL; between about 1 mg/mL and about 10 mg/mL; between about 2 mg/mL and about 9 mg/mL; between about 3 mg/mL and about 8 mg/mL; between about 4 mg/mL and about 7 mg/mL; between about 4 mg/mL and about 6 mg/mL. In certain embodiments, compositions are formulated such that the compound is present at a concentration of about 5 mg/mL.

Kits

The invention also provides compositions and kits for prognosing the ability of a compound that alters the activity of the iRhom polypeptide to accelerate wound healing, treat cancer, or treat an inflammatory disease in a subject or for determining whether a cancer in a subject is sensitive to treatment with the compound. These kits include one or more of the following: reagents for obtaining and/or preparing samples, e.g., skin biopsy, tumor biopsy or blood samples; reagents for determining whether a sample exhibits iRhom proteolytic activity; probes and reagents for determining whether a sample exhibits a mutation in a gene, e.g., the Rhbdf1 or Rhbdf2 gene; probes and reagents for determining whether a sample exhibits a wild-type sequence of a gene, e.g., the Rhbdf1 or Rhbdf2 gene; reagents for determining the half-life of an iRhom polypeptide in a sample; reagents for determining the secretion of EGFR ligands by a sample; and instructions for use.

The kits of the invention may optionally comprise additional components useful for performing the methods of the invention. By way of example, the kits may comprise fluids (e.g., SSC buffer) suitable for annealing complementary nucleic acids or for binding an antibody with a protein with which it specifically binds, one or more sample compartments, an instructional material which describes performance of a method of the invention, a sample of normal cells, a sample of cancer cells, a sample of wounded cells, a sample of inflamed cells and the like.

Indications

Wound Healing

Wound healing is a process whereby the skin or another organ-tissue repairs itself after injury. In one embodiment, the present invention may be used to identify a compound that activates the proteolytic site of the iRhom polypeptide and thereby accelerate wound healing in a subject.

The methods of the invention may be used to identify a compound that accelerates wound healing in a subject. In one embodiment, the compound accelerates wound healing such that wound closure is achieved. In one embodiment, the wound may be an open cutaneous wound, such as a burn wound, a wound resulting from chemical (e.g., alkali) burn, a wound from physical trauma, neuropathic ulcers, pressure sores, venous stasis ulcers, and diabetic ulcers.

In another embodiment, the present invention provides compounds or pharmaceutical compositions that activate the proteolytic site of an iRhom polypeptide to accelerate, promote or enhance wound healing in a subject.

In some embodiments, the present invention provides for the use of any of the compounds described above or elsewhere herein to activate the proteolytic site of an iRhom polypeptide to accelerate healing of a wound or modulate one or more properties of cells in the wound environment or in the immediate vicinity of the wound. In some embodiments, the present invention provides for the use of the compositions described above to enhance healing of a wound or modulate one or more properties of cells in the wound environment or in the immediate vicinity of the wound.

Cancer

Compounds of the invention may be useful for treating cancer in a subject. The compounds of the invention may also be useful in reducing tumor growth and/or progression in a subject.

In one embodiment, the present invention may be used to identify a compound capable of reducing tumor growth and/or progression or treating cancer in a subject. In one embodiment, the compound inhibits the proteolytic site of the iRhom polypeptide and thereby decreases secretion of a physiological target of the iRhom polypeptide and/or decreases EGFR activity, and thereby reduces tumor growth and/or progression. In another embodiment, the compound inhibits the proteolytic site of the iRhom polypeptide and thereby decreases secretion of a physiological target of the iRhom polypeptide and/or decreases EGFR activity, and is thereby useful in treating cancer.

In some embodiments, missense mutations in RHBDF2 (p.I186T; p.P189L; p.D188N), the gene encoding iRhom2, may result in tylosis with human esophageal cancer, which is characterized by palmoplantar and oral hyperkeratosis.

The compounds of the invention may be useful in treating various cancers. In one embodiment, the cancer is an epithelial cancer. In another embodiment the cancer is cancer of the esophagus, lung, brain, colon, kidney, prostate, skin, liver, pancreas, stomach, uterus, ovary, breast, lymph glands or bladder.

The compounds of the invention may also be useful in treating various tumors. In one embodiment the tumor is a solid tumor.

Inflammation

Compounds of the invention may be useful for reducing inflammation in a subject. The compounds of the invention may be useful in treating inflammatory diseases and/or disorders. Inflammatory diseases and/or disorders, include, for example, systemic lupus erythematosus (SLE), lupus nephritis, cryoglobulinemia, vasculitis, rheumatoid arthritis, Sjoegren's syndrome, uveitis, Spondyloarthritis, miscarriage, preeclampsia, acne vulgaris, asthma, autoimmune diseases, celiac disease, chronic prostatitis, glomerulonephritis, hypersensitivities, inflammatory bowel diseases (including ulcerative colitis and Crohn's disease), Pelvic inflammatory disease, Reperfusion injury, Rheumatoid arthritis, Sarcoidosis, Transplant rejection, Vasculitis, Interstitial cystitis, Atherosclerosis, Allergies, Myopathies, leukocyte defects, psoriasis, multiple sclerosis and cancer

In one embodiment, the present invention may be used to identify a compound that is capable of reducing inflammation or treating an inflammatory disorder in a subject. In one embodiment the compound reduces the activity of proteolytic domain of the iRhom polypeptide such that there is a decrease in the secretion of a physiological target of the iRhom polypeptide and/or a decrease in TNFα secretion and thereby a decrease in inflammation. In another embodiment the compound reduces the activity of proteolytic domain of the iRhom polypeptide such that there is a decrease in the secretion of a physiological target of the iRhom polypeptide and/or a decrease in TNFα secretion and is thereby useful in treating an inflammatory disorder or disease. In yet anther embodiment, the compound of the invention reduces TNFα secretion by targeting the cytosolic domain or transmembrane peptidase domain, and is thereby useful to reduce inflammation in the subject.

Various tests can be employed to assay the efficacy of a compound at treating an inflammatory disorder.

Hair Growth

Compounds of the invention may be useful for treating baldness and/or promoting hair growth in the subject. Hair loss or baldness (e.g., alopecia) is a loss of hair from the head or body. Baldness can refer to general hair loss, male pattern baldness, or hair loss in women. Some types of baldness can be caused by alopecia areata, an autoimmune disorder. The extreme forms of alopecia areata are alopecia totalis, which involves the loss of all head hair, and alopecia universalis, which involves the loss of all hair from the head and the body.

In one embodiment, the present invention is based on the discovery that the cub mutation causes balding. In another embodiment, the present invention is based on the discovery that the modifier of cub (Mcub) promotes hair growth, cures balding, corrects balding, or reduces balding (Example 1).

In one embodiment, the present invention may be used to identify a compound capable of promoting hair growth in a subject comprising: contacting a cell expressing an iRhom polypeptide with a test compound; and determining a decrease in secretion of a physiological target of the iRhom polypeptide relative to an appropriate control or a decrease in EGFR activity relative to an appropriate control, wherein a decrease in secretion of a physiological target of the iRhom polypeptide or a decrease in EGFR activity indicates that the compound is capable of promoting hair growth in the subject.

In another embodiment, the present invention may be used to identify a compounds capable of curing and/or correcting baldness in a subject comprising: contacting a cell expressing an iRhom polypeptide with a test compound; and determining a decrease in secretion of a physiological target of the iRhom polypeptide relative to an appropriate control or a decrease in EGFR activity relative to an appropriate control, wherein a decrease in secretion of a physiological target of the iRhom polypeptide or a decrease in EGFR activity indicates that the compound is capable of curing and/or correcting baldness in the subject.

This invention is further illustrated by the following examples which should not be construed as limiting. The entire contents of all references, patents and published patent applications cited throughout this application are hereby incorporated herein by reference

### Examples

### Example 1: The Cub Mutation Leads to Hyperactivation of the EGFR Signaling Pathway

The recessive mouse mutation named curly-bare (cub) is characterized by a hairless phenotype. A single copy of the dominant Mcub allele in combination with the cub/cub genotype results in a full, wavy coat rather than the hairless coat of cub/cub mcub/mcub mice. We investigated whether the cub/cub mcub/mcub genotype might be associated with aspects of altered EGFR signaling such as cell proliferation and cell migration.

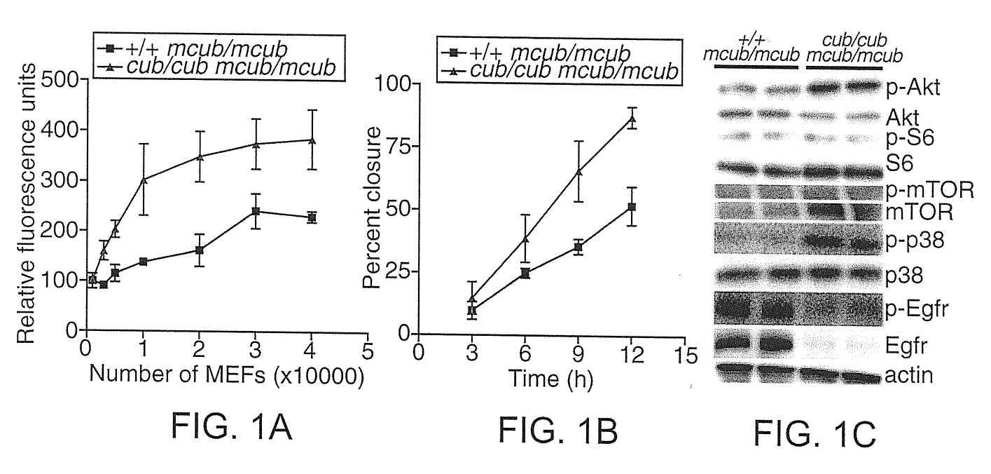

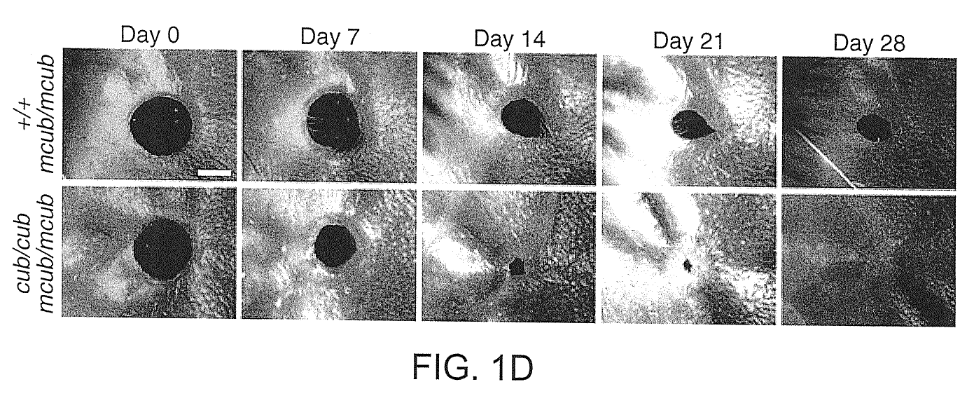

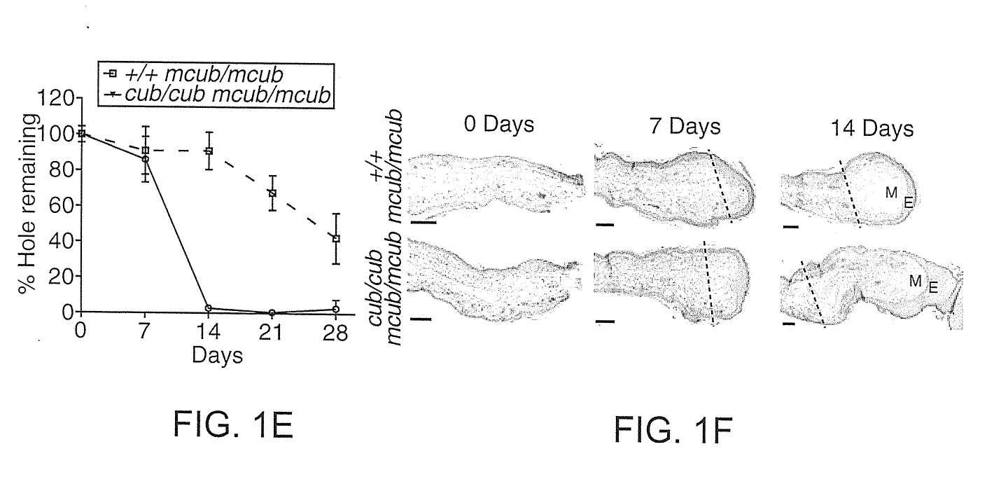

We performed proliferation, cell migration (scratch-wound healing), and immunoblot assays on mouse embryonic fibroblasts (MEFs) isolated from cub/cub mcub/mcub and control (+/+ mcub/mcub) mice. FIGS. 1A-1F show that the cub mutation accelerates EGFR-related cell proliferation and migration along with cutaneous healing.

The cub/cub mcub/mcub MEFs had significantly higher rates of proliferation and migration relative to control MEFs (FIGS. 1A and 1B). These changes were associated with significant increases in the phosphorylation of canonical signal transduction proteins of the EGFR pathway, including, Akt, S6, mTOR, and p38 (FIG. 1C). Further, we observed a significant reduction in cell-surface EGFR levels in cub/cub mcub/mcub MEFs (FIG. 1C), indicating internalization and constitutive activation of EGFR signaling.

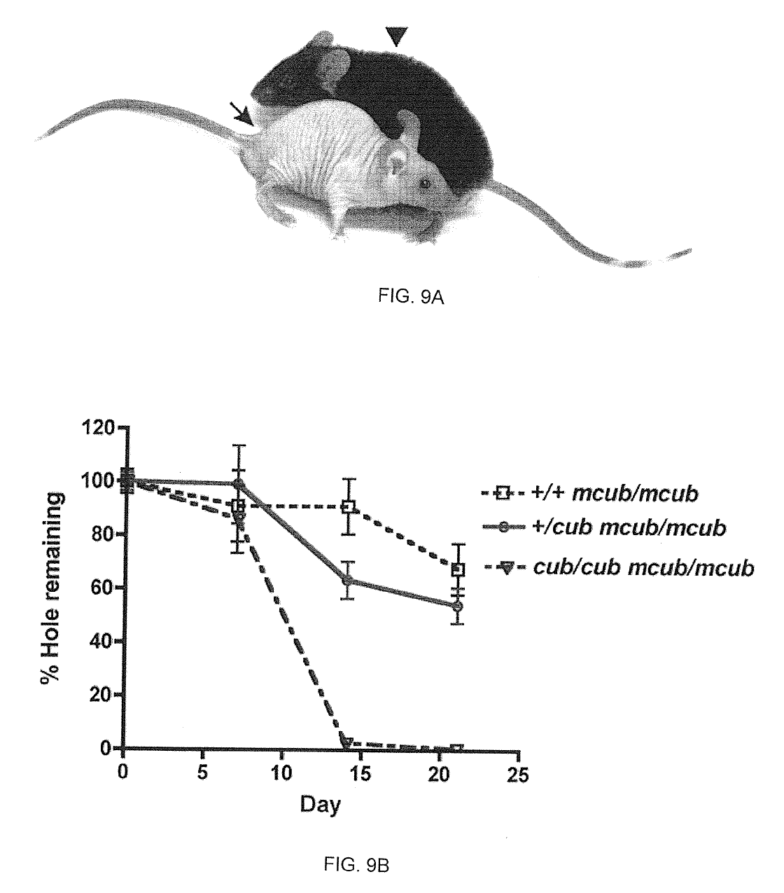

To test whether an increase in EGFR signaling corresponds to changes in epithelial proliferation, we conducted wound-healing assays in which we punched 2-mm through-and-through holes into the ears of 6-40-week-old cub mice and monitored the rate of closure over subsequent days (FIGS. 1D-1F). Within 14 days, cub/cub mcub/mcub mice showed accelerated closure compared with control +/+ mcub/mcub littermates. The +/cub mcub/mcub mice (which have a normal coat) also showed faster healing 14 days post-injury relative to control mice, see FIGS. 9A and 9B, indicating that a single mutant cub allele can trigger increased EGFR signaling, although not at levels high enough to block hair follicle induction. These data indicate that the cub/cub mcub/mcub genotype results in a hyperactive EGFR phenotype.

### Example 2: Cub is a Mutation of the Rhbdf2 Gene, Encoding iRhom2

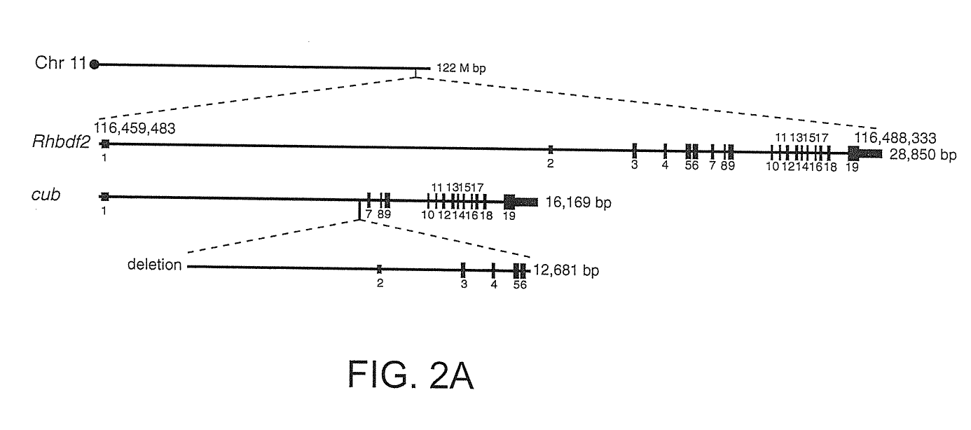

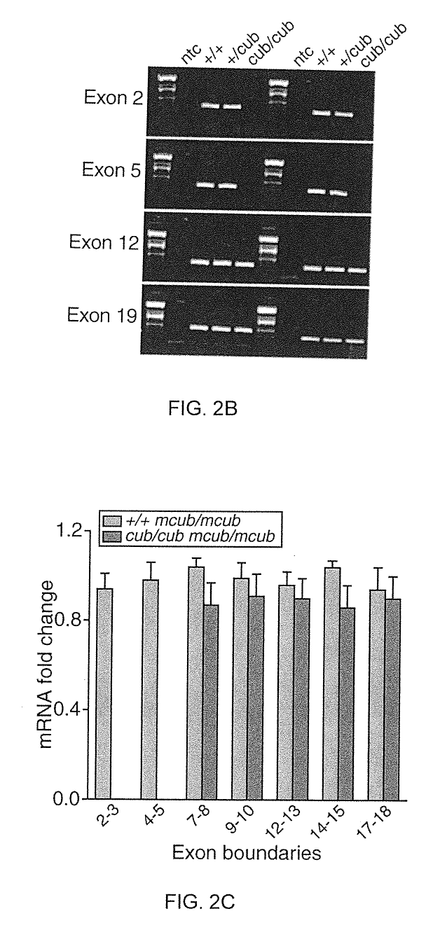

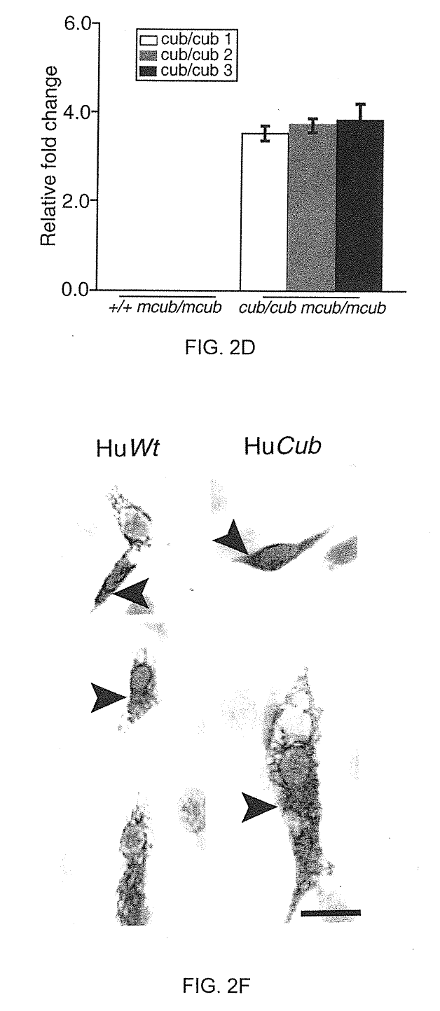

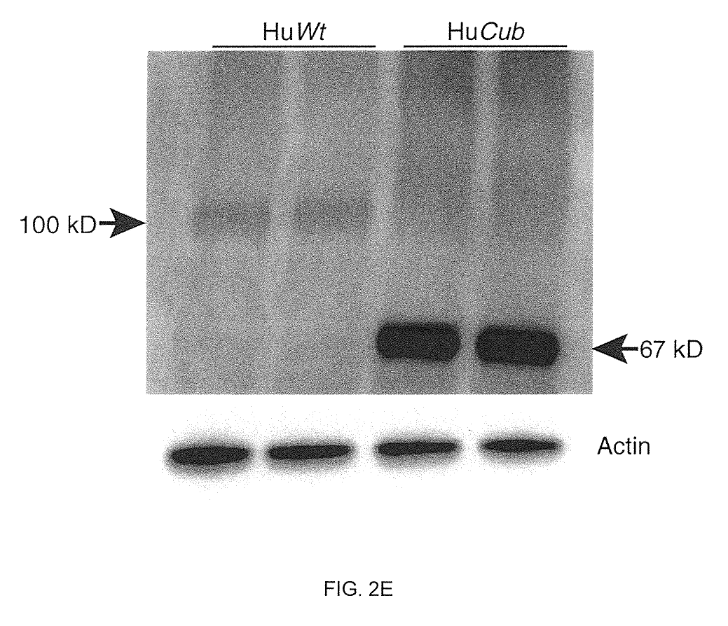

Next, we examined the iRhom2 gene Rhbdf2 as a candidate for the cub mutation. DNA sequencing of cub/cub mice identified a 12,681 base pair (bp) deletion in the Rhbdf2 gene, which results in loss of exons 2-6 (FIG. 2A). To test whether the deletion produces an aberrant Rhbdf2 transcript, we performed reverse-transcriptase PCR (RT-PCR) on RNA derived from wildtype and cub/cub mcub/mcub MEFs using primers designed to amplify exons 2, 5, 12 and 19. The cub transcript contained exons 12 and 19, but lacked exons 2 and 5 (FIG. 2B). Further, quantitative RT-PCR (qPCR) on RNA from cub/cub mcub/mcub and +/+ mcub/mcub skin amplified cub transcripts in which exons 2-6 were deleted but the remaining exons were expressed, indicating that the rest of the cub gene is transcribed in cub mice (FIG. 2C). To test whether the cub transcripts would result in splicing of exons 1 to 7, we performed qPCR with a probe specifically designed to amplify only transcripts containing exon 1 spliced to exon 7. Using this probe, we detected a PCR product in cub/cub mcub/mcub mice but not in +/+ mcub/mcub mice (FIG. 2D). These findings confirm that cub is a mutation of the Rhbdf2 gene, and will henceforth be referred to as Rhbdf2^{cub}.

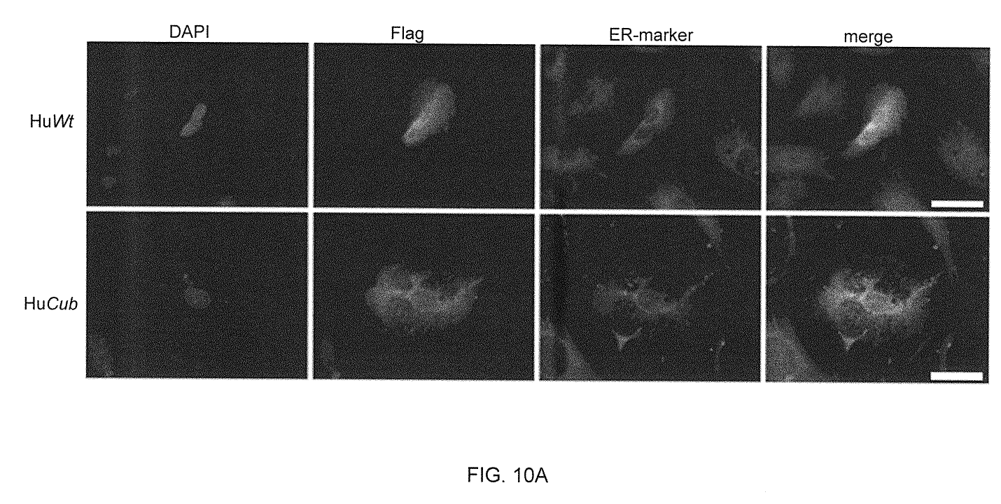

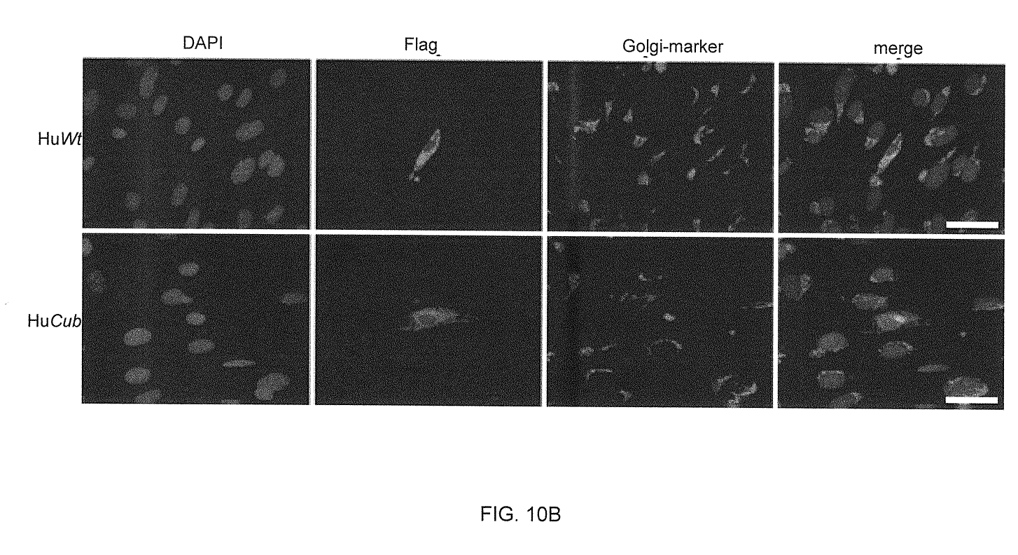

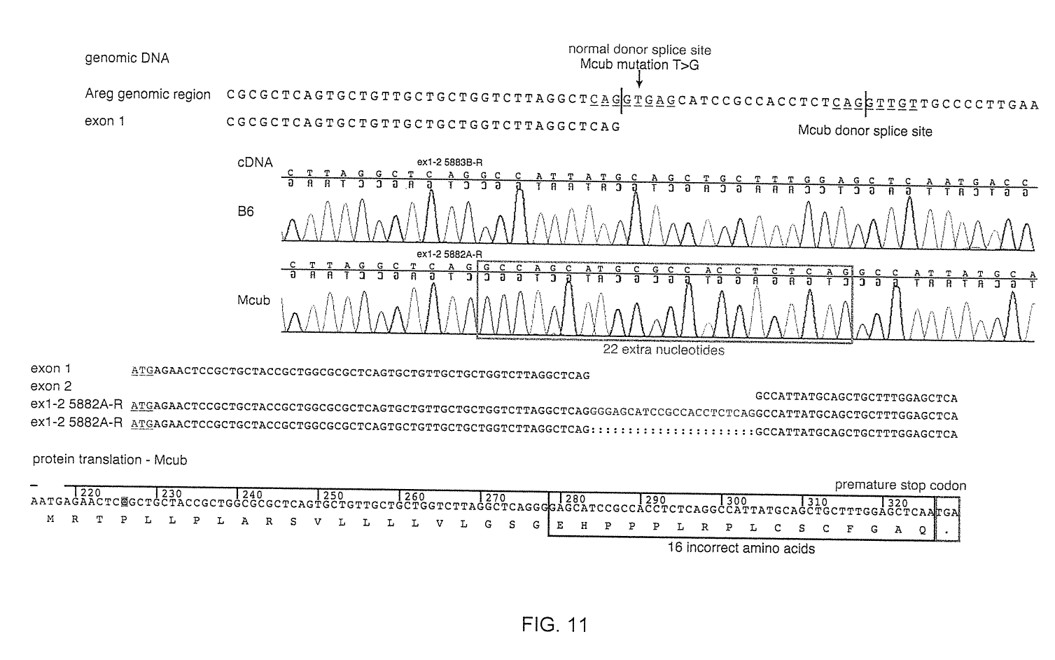

Given that the normal translation initiation site in exon 3 is missing in Rhbdf2^{cub} transcripts, we next tested whether these mutant transcripts could produce a mutant iRhom2 protein. Sequence analysis of Rhbdf2^{cub} DNA revealed that the next in-frame translation initiation site (ATG) was in exon 8, which would result in a ˜63.5 kDa protein. Since we lacked an antibody to iRhom2, we tested whether Rhbdf2^{cub} transcripts could produce a protein product in vitro by cloning both full-length wildtype human RHBDF2 cDNA (HuWt) as well as a version that mimicked the mutant Rhbdf2^{cub} transcript (HuCub) into a C-terminal Flag-tagged expression vector. First, using immunoblotting, we determined that the HuWt clone generated a ˜100 kDa protein product, whereas the HuCub mutant construct generated a ˜67 kDa product, consistent with the expected molecular weight of the tagged proteins (FIG. 2E). Furthermore, there was no evidence of any shortened protein products in the HuWt clone. Second, using immunostaining, we examined the localization patterns of both forms of protein after transfection of B6 MEFs (FIG. 2F and FIGS. 10A and 10B) with either the HuWt or HuCub clones. Both forms were expressed in the endoplasmic reticulum, and no staining was observed in either the Golgi or nucleus (FIGS. 10A and 10B), indicating that the mutation does not lead to altered protein localization.

### Example 3: Genetic Non-Complementation Confirms that Cub is a Mutant Allele of the Rhbdf2 Gene and a Gain-of-Function Mutation

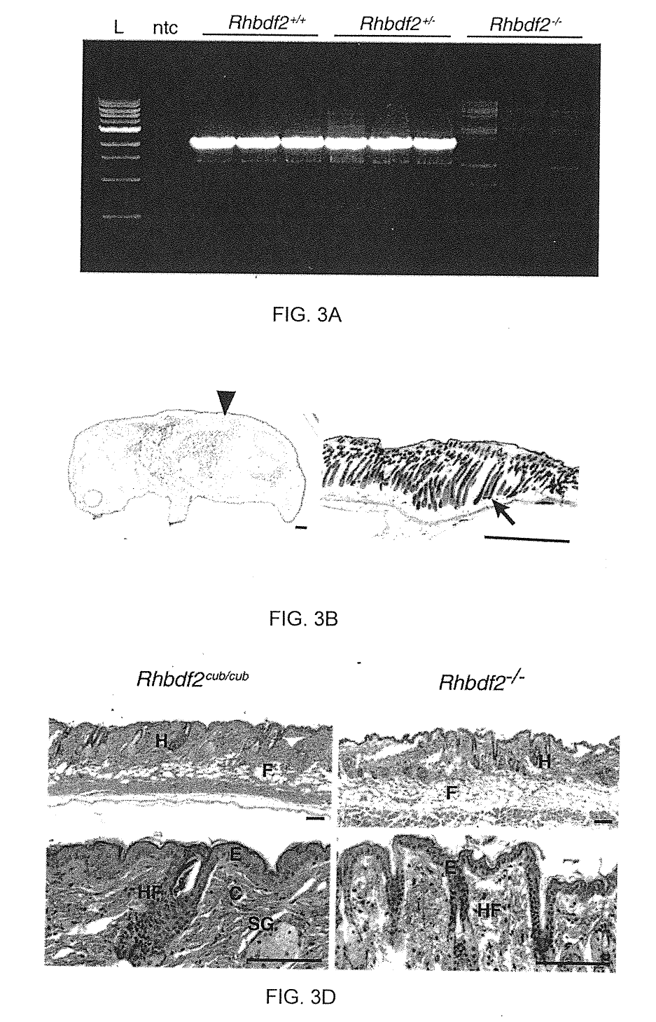

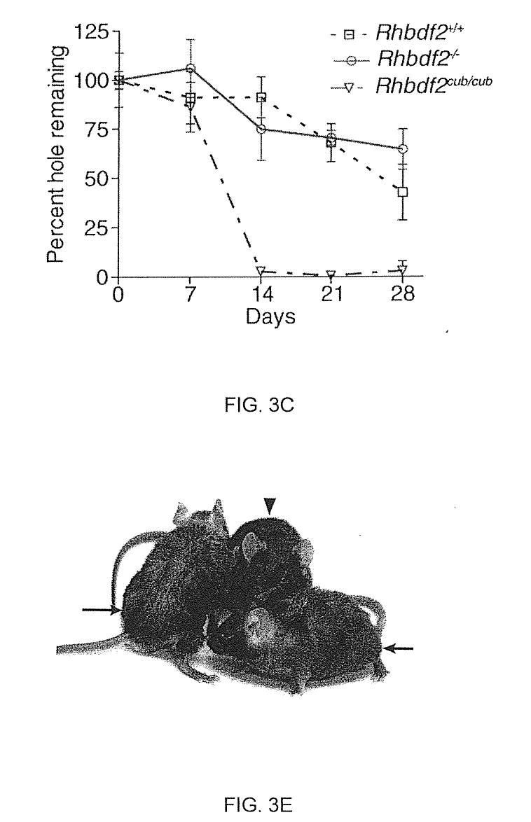

To test whether Rhbdf2^{cub} is a gain-of-function rather than a null mutation, we generated Rhbdf2 knockout (Rhbdf2^{-/-}) mice using ES cells from the Knockout Mouse Project (KOMP) repository, in which lacZ expression is under control of the endogenous Rhbdf2 promoter (FIG. 3A). Rhbdf2 promoter-driven lacZ expression was predominantly observed in the epidermis and the inner and outer sheath layers of hair follicles in the skin (FIG. 3B). However, the wound healing (FIG. 3C) and loss-of-hair (FIG. 3D) phenotypes observed in the Rhbdf2^{cub/cub} mice were not seen in Rhbdf2^{-/-} mice, which appeared otherwise normal. An allele-test mating between Rhbdf2^{cub/cub} and Rhbdf2^{-/-} mice yielded compound mutant (Rhbdf2^{-/cub}) mice with a sparse hair coat (FIG. 3E), indicating genetic non-complementation, and confirming that cub is a mutant allele of the Rhbdf2 gene. Additionally, the level of hair growth in the Rhbdf2^{-/cub} compound mutant mice was intermediate between Rhbdf2^{cub/cub} and Rhbdf2^{+/+} or Rhbdf2^{-/-} mice, confirming that expression of the mutant Rhbdf2^{cub} protein product, rather than Rhbdf2 deficiency, causes the Rhbdf2^{cub/cub} phenotype.

### Example 4: Genetic Modifier of the Rhbdf2^{cub} Phenotype (Mcub) is a Loss-of-Function Mutation of the Areg Gene

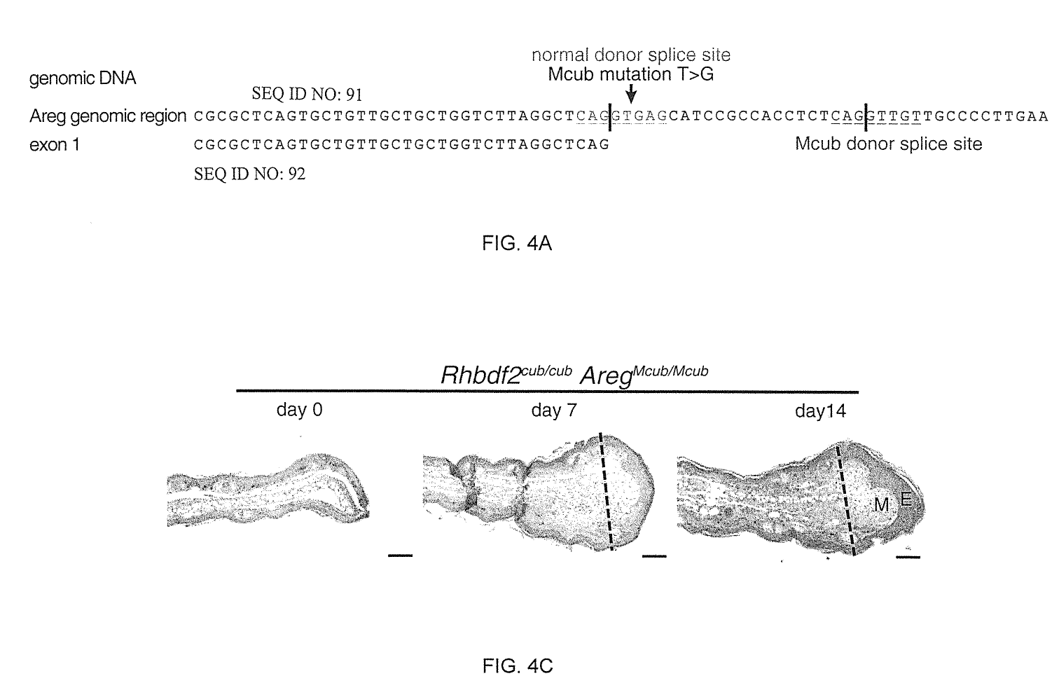

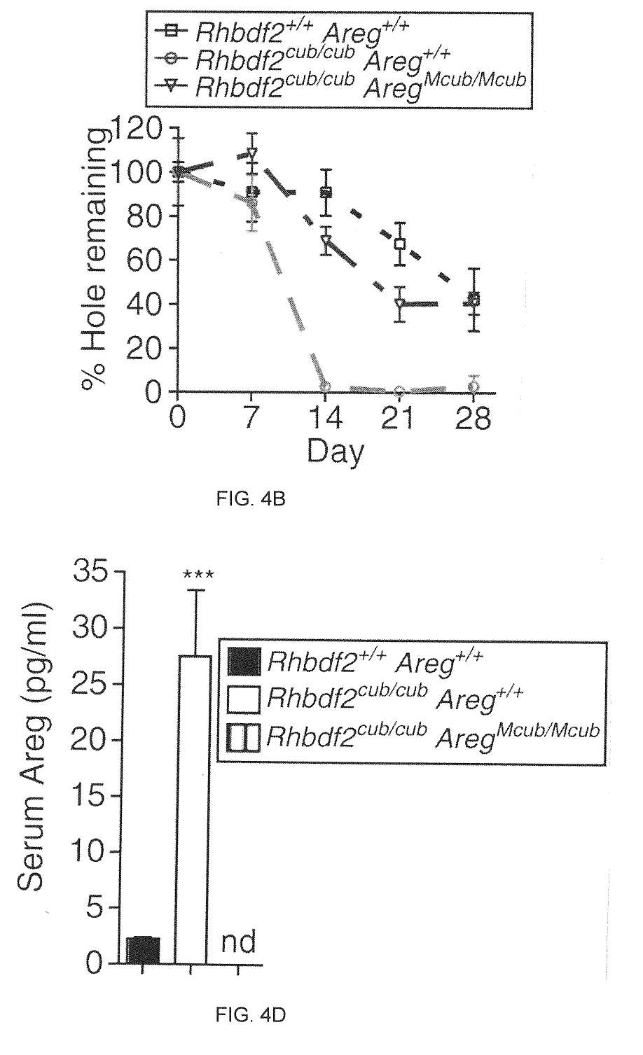

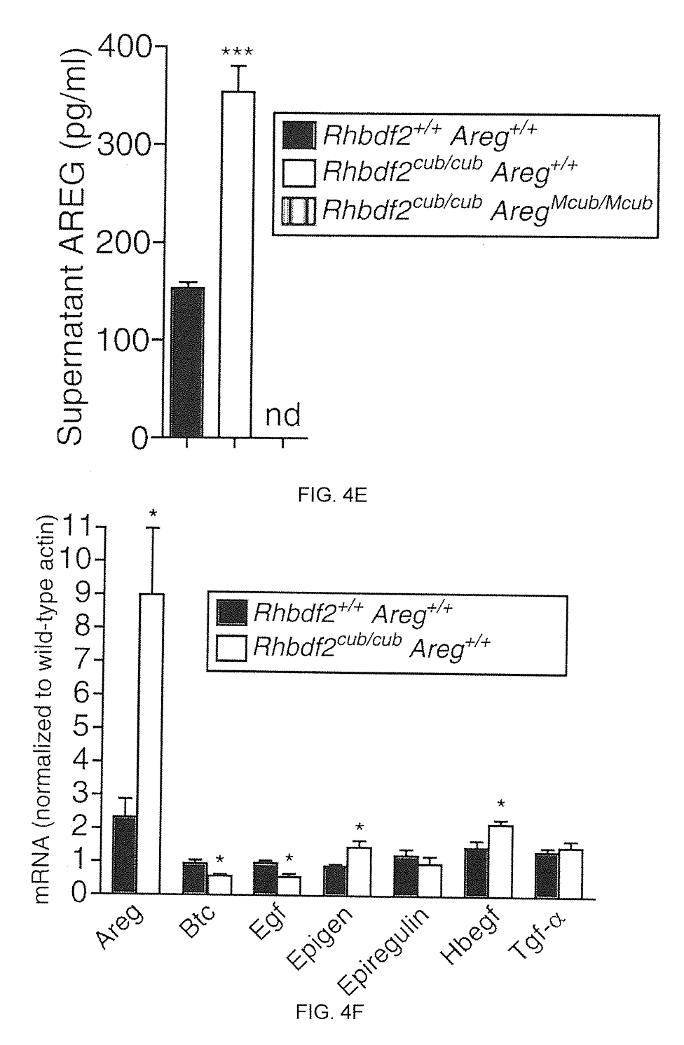

We next sought to map the mutation underlying the Rhbdf2^{cub} modifier gene (Mcub). We examined four EGFR ligand-encoding genes (Epng, Ereg, Areg, and Btc) as candidate loci for the Mcub mutation. By sequencing the exons and flanking regions of each gene in Mcub/Mcub Rhbdf2^{cub/cub} mice, see Table II, we found that Mcub is a loss-of-function mutation in Areg, FIG. 4A, a gene that encodes the autocrine keratinocyte growth factor amphiregulin (herein called AREG).

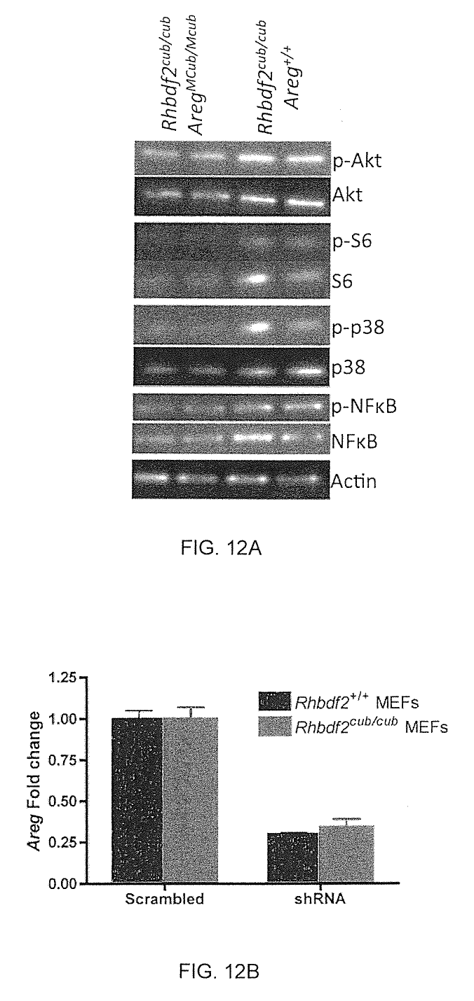

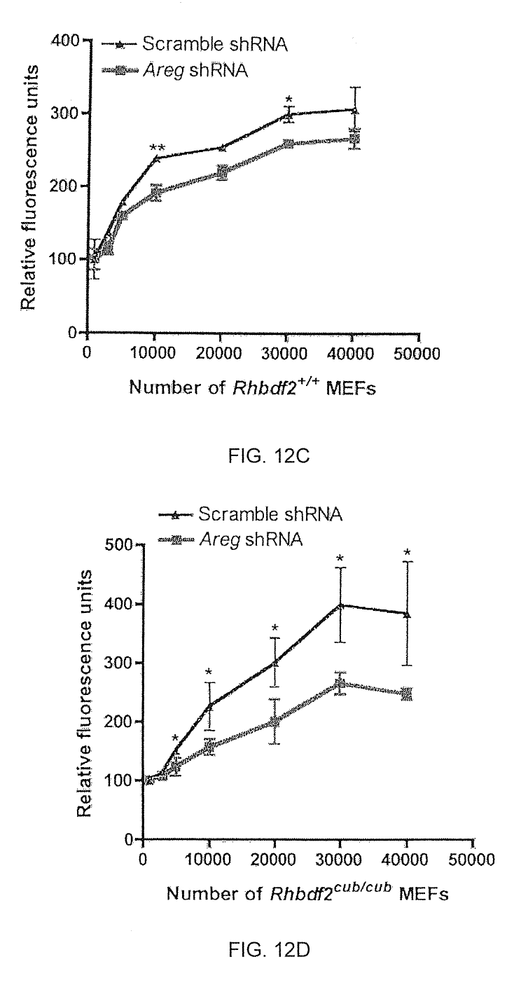

Mcub is a T-to-G point mutation that destroys the canonical donor splice site of exon 1 and leads to the exclusive use of an alternative downstream splice site that adds 22 extra nucleotides to the Areg transcript; this disrupts the coding frame and introduces a premature stop codon (FIG. 1). This mutation will henceforth be referred to as Areg^{cub}. Notably, the hyperactive EGFR signaling (FIG. 12A) and the rapid wound closure capability of Rhbdf2^{cub/cub} mice is significantly reduced (FIGS. 4B and 4C), and the loss-of-hair phenotype prevented when a single copy of the dominant Areg^{Mcub} allele is present. The dominant Areg^{Mcub} mutation does not confer a normal hair coat to the Rhbdf2^{cub/cub} mice, but rather a wavy hair phenotype, indicating remaining abnormalities in the EGFR pathway.

We next measured serum levels of AREG in Rhbdf2^{cub/cub} Areg^{+/+}, Rhbdf2^{cub/cub} Areg^{Mcub/Mcub}, and Rhbdf2^{+/+} Areg^{+/+} mice. We found no detectable AREG in the serum of Rhbdf2^{cub/cub} Areg^{Mcub/Mcub} mice, but did observe a dramatic increase in serum AREG levels in Rhbdf2^{cub/cub} Areg^{+/+} mice compared with Rhbdf2^{+/+} Areg^{+/+} mice (FIG. 4D).

Measurements of supernatant AREG levels from cultured mouse epidermal keratinocytes (MEKs) of Rhbdf2^{cub/cub} Areg^{+/+}, Rhbdf2^{cub/cub} Areg^{Mcub/Mcub}, and Rhbdf2^{+/+} Areg^{+/+} mice yielded similar results (FIG. 4E). AREG is abundantly expressed in normal skin; therefore, we next performed qPCR on skin samples from Rhbdf2^{cub/cub} Areg^{+/+} and Rhbdf2^{+/+} Areg^{+/+} mice to measure transcript levels of Areg, as well as six other genes known to encode EGFR ligands (Egf, Tgfa, Btc, Epgn, Ereg and Hbegf). Compared to controls, we observed a four-fold increase in Areg, and subtle but statistically significant increases in Epgn and Hbegf mRNAs, in Rhbdf2^{cub/cub} Areg^{+/+} mice. Also, there was a statistically significant decrease in Btc and Egf transcript levels (FIG. 4F).

Lastly, to examine whether AREG mediates hyperactive EGFR phenotype, we silenced Areg expression in Rhbdf2^{+/+} and Rhbdf2^{cub/cub} MEFs (FIG. 12B) using lentiviral shRNA and performed proliferation assays. While silencing of Areg had a subtle effect on proliferation of Rhbdf2^{+/+} MEFs (FIG. 12C), proliferation rates of Rhbdf2^{cub/cub} MEFs were significantly reduced (FIG. 12D). Taken together, these data suggest that enhanced AREG levels mediate the mutant phenotype of the Rhbdf2^{cub/cub} mice via a gain of EGFR signaling. Furthermore, our data reveal that the Rhbdf2^{cub/cub} phenotype is modified by Areg^{Mcub} expression.

### Example 5: The Peptidase Domain of the N-Terminal-Truncated iRhom2 (Rhbdf2^{cub}) Induces Substrate-Specific Secretion of EGFR Ligands Independent of ADAM17

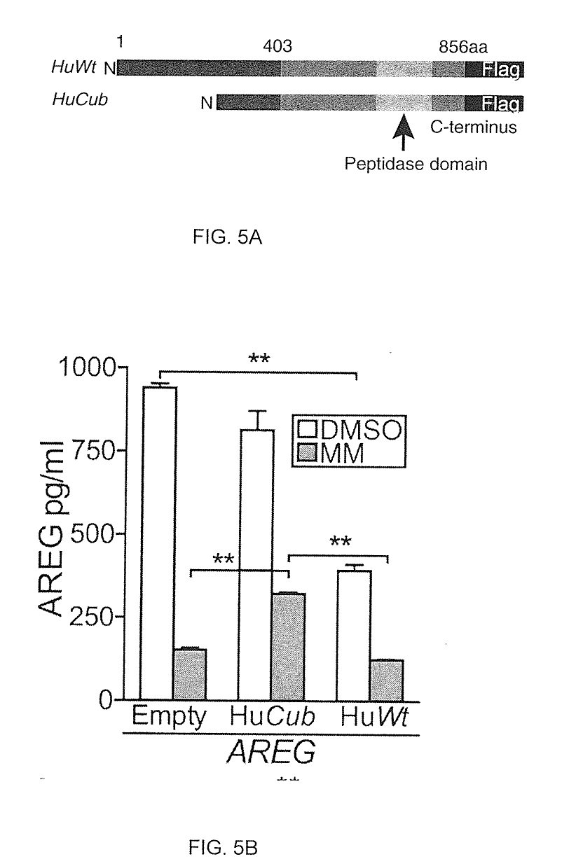

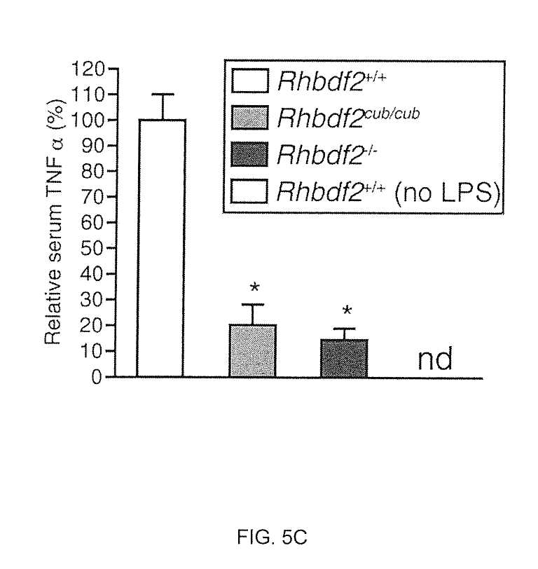

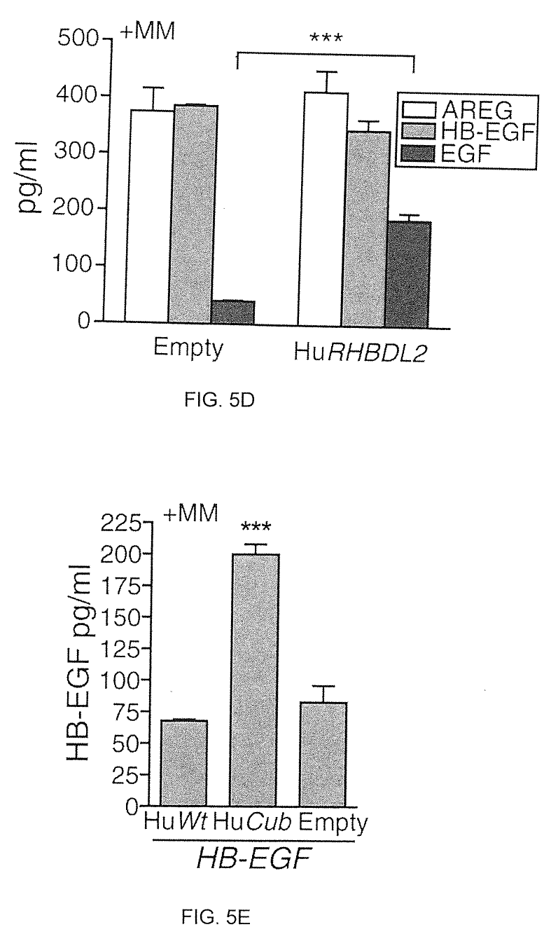

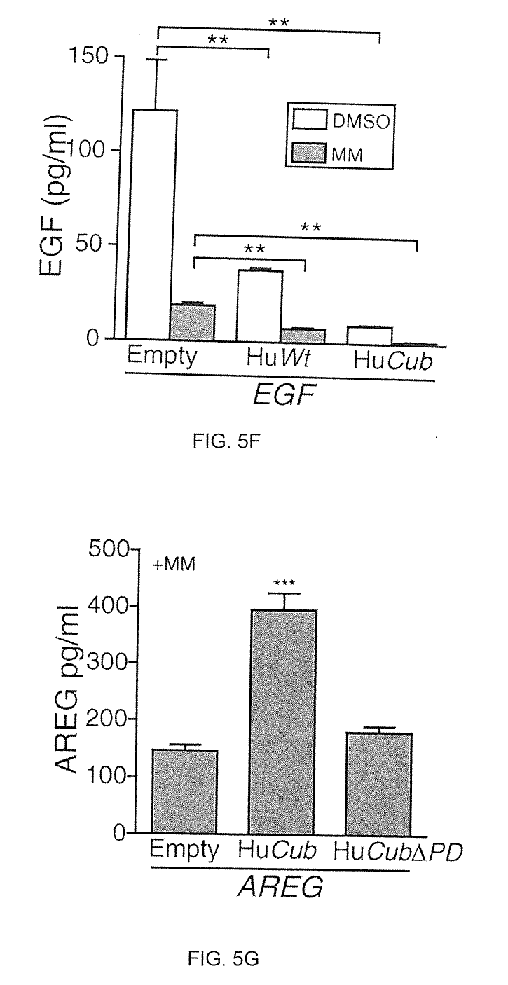

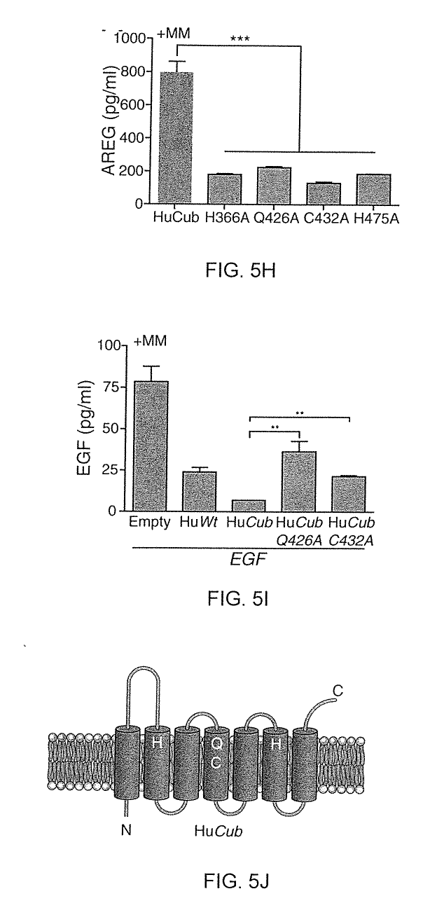

To test whether Rhbdf2^{cub}, with its short N-terminal domain, could induce secretion of AREG independently of metalloprotease activity, we performed in vitro cleavage assays in the presence or absence of marimastat (MM), a potent broad-spectrum metalloprotease inhibitor that can block both ADAM17- and ADAM10-dependent shedding of substrates. Because of the significant homology between the mouse and human RHBDF2 genes we transfected 293T cells with human AREG either alone or with the human wildtype (HuWt) or human Cub (HuCub) RHBDF2 genes (FIG. 5A), and measured AREG levels in conditioned medium. In the absence of MM, we observed no difference in AREG levels between AREG-expressing and AREG/HuCub co-expressing cells, whereas co-expression of HuWt and AREG reduced AREG levels by ˜60% (FIG. 5B). In the presence of MM, AREG levels were approximately twofold higher in HuCub/AREG co-transfected cells compared with cells transfected with HuWt and AREG or AREG alone (FIG. 5B). These data suggest that N-terminal-truncated iRhom2 can enhance AREG secretion, which only becomes apparent when metalloprotease activity is diminished.

ADAM17 activity can be measured in mice by examining tumor necrosis factor alpha (TNFα) secretion after stimulation with bacterial endotoxin lipopolysaccharide (LPS). We used this approach to test changes in ADAM17 activity in Rhbdf2^{cub} mice, by injecting LPS into Rhbdf2^{cub/cub}, Rhbdf2^{-/-} and Rhbdf2^{+/+} mice and measuring serum TNFα levels. We found that both LPS-injected Rhbdf2^{cub/cub} and Rhbdf2^{-/-} mice had a markedly lower induction of TNFα relative to LPS-injected Rhbdf2^{+/+} mice (FIG. 5C). These results suggest that ADAM17 activity is significantly attenuated in Rhbdf2^{cub/cub} and Rhbdf2^{-/-}, and also implicate a role for the N-terminal domain of iRhom2 in regulation of ADAM17-dependent TNFα release. Notably, serum TNFα levels in LPS-stimulated Rhbdf2^{cub/cub} mice were not completely abrogated, indicating that ADAM17 activity is attenuated but not eliminated. The observation that ADAM17 activity is attenuated in Rhbdf2^{cub/cub} mice explains the non-lethal or non-inflammatory cutaneous phenotype in Rhbdf2^{-/-} and Rhbdf2^{cub/cub} mice compared with Adam17^{-/-} mice or with transgenic mice overexpressing amphiregulin.

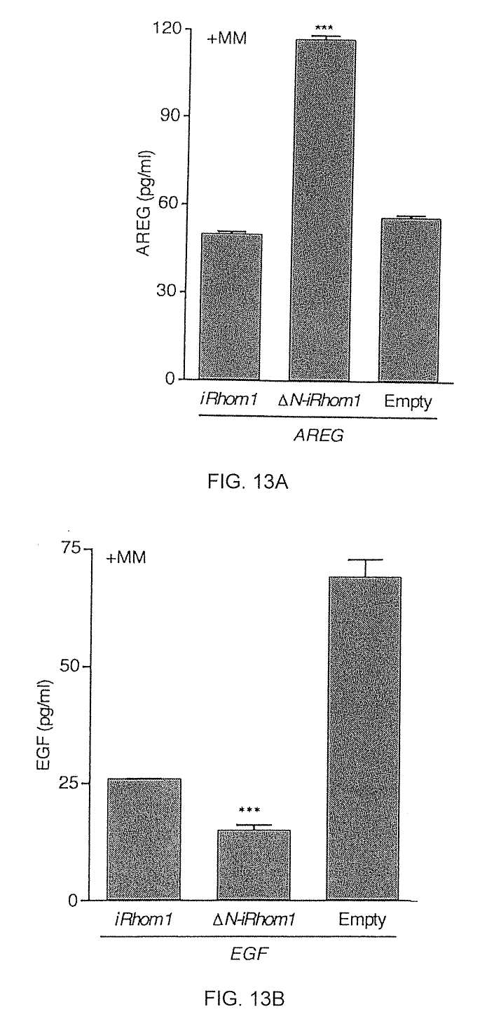

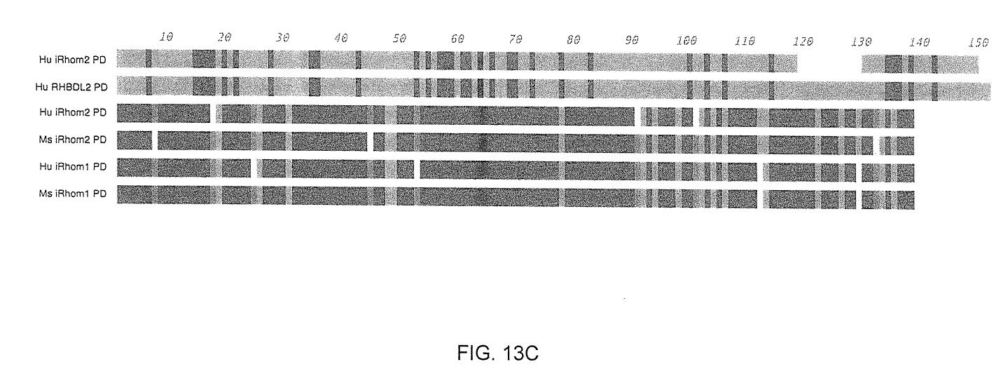

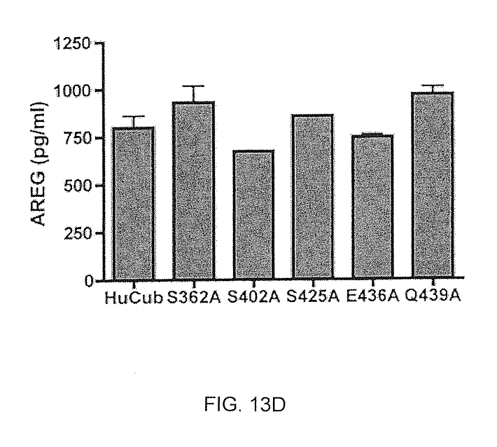

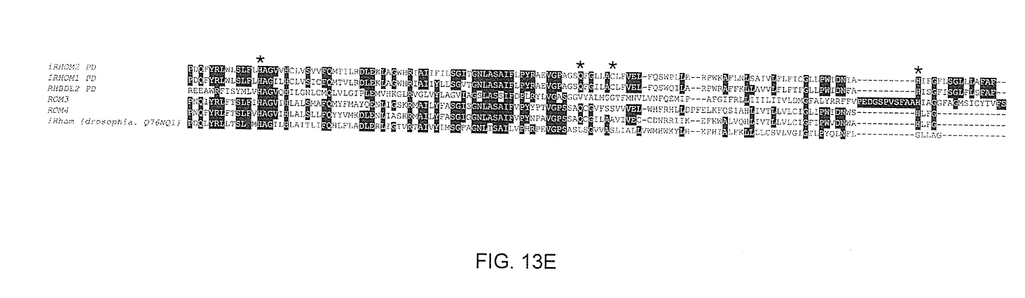

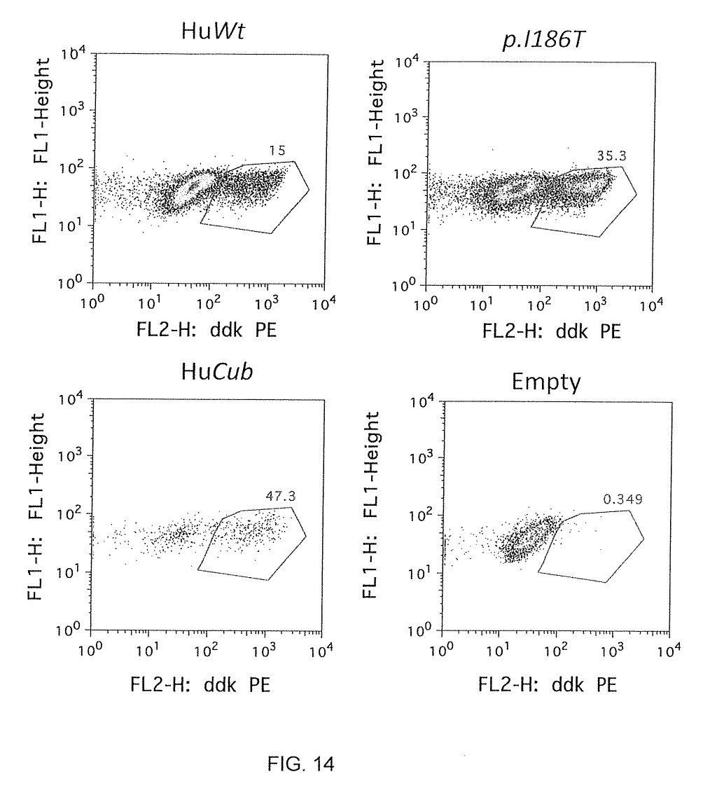

Next, the protease selectivity of the active rhomboid proteases for other EGF-like substrates, and whether these differed from HuCub, was assessed. We found that RHBDL2 selectively increased secretion of EGF but not AREG or HB-EGF compared with empty vector transfected controls (FIG. 5D). In contrast, HuCub selectively increased secretion of AREG and HB-EGF but not EGF compared with empty vector transfected cells (FIGS. 5E-5G). These results suggest that the rhomboid peptidase domain confers substrate selectivity. The ability of N-terminal-truncated iRhom1 to induce AREG but not EGF secretion is consistent with this assertion (FIGS. 13A and 13B).

To determine how the iRhom2 peptidase domain helps to regulate the secretion of EGF-like substrates, we initially aligned sequences for amino acids of the peptidase domains of human and mouse iRhoms. We found a significant difference in protein sequence homology between the peptidase domains of human iRhom2 and RHBDL2 (FIG. 13C). By contrast, the peptidase domains of iRhom1 and 2 show a 96% homology. Together, this suggests that the residues that form the active site or that play a role in substrate recognition differ significantly between active and inactive rhomboids. In addition, we found that deletion of the HuCub peptidase domain significantly diminished AREG secretion compared with native HuCub (FIG. 5G), indicating that the peptidase domain is essential for secretion of EGF-like substrates. To identify the critical residues, we performed site-directed mutagenesis such that key residues in the HuCub peptidase domain were mutated to alanines. We found that glutamine-426 and cysteine-C432 in transmembrane domain four, and histidines 366 and 475 in transmembrane domains two and six, respectively, were critical not only for mediating enhanced secretion of AREG/HB-EGF but also for suppression of EGF (FIG. 5H, I). However, mutations in serines S362A, 402A and 425A, glutamic acid E436A, and glutamine Q439A did not alter AREG secretion (FIG. 13D). Together, these results suggest that key residues (FIG. 5J & FIG. 13E) in the peptidase domain of N-terminal-truncated iRhom regulate the secretion of EGF-like substrates independent of metalloprotease activity.

### Example 6: The N-Terminal-Truncated iRhom2 (Rhbdf2^{cub}) Increases Susceptibility to Epithelial Cancers

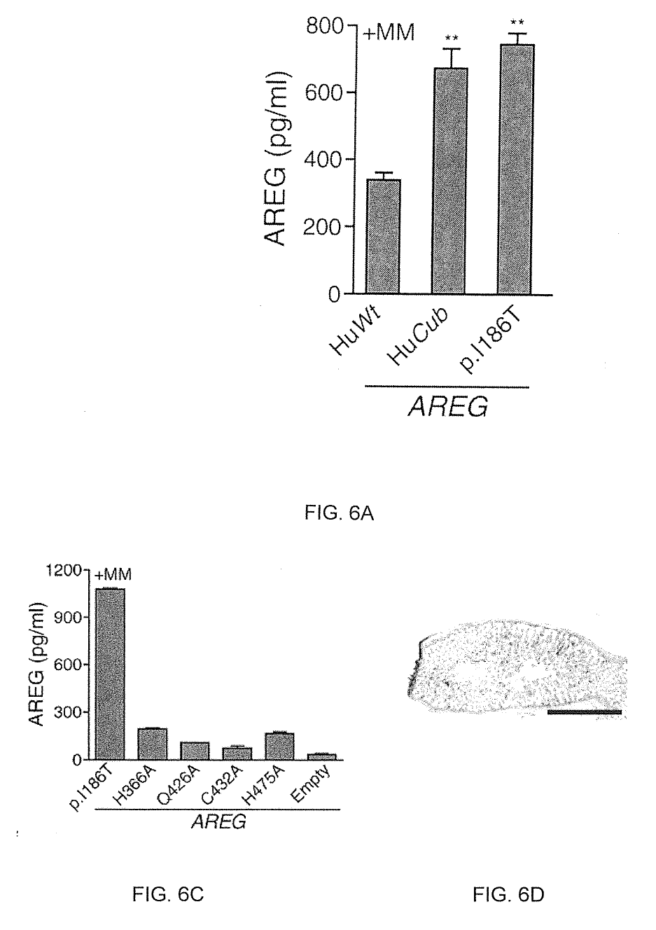

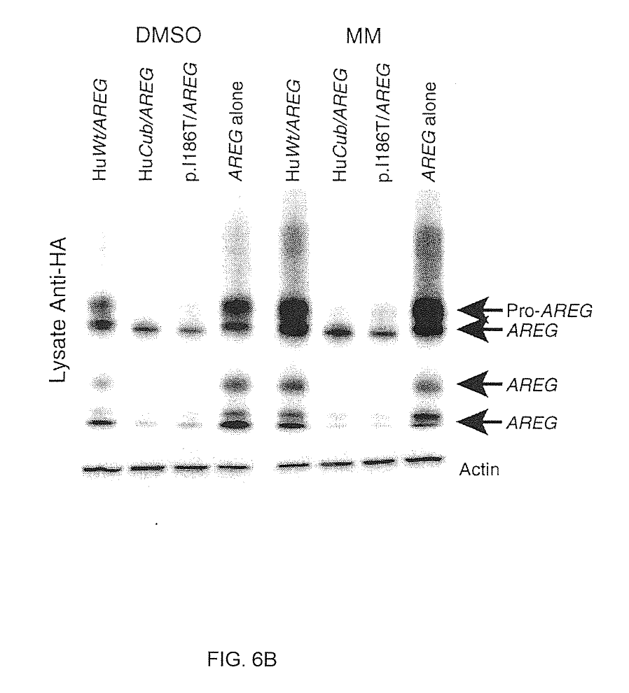

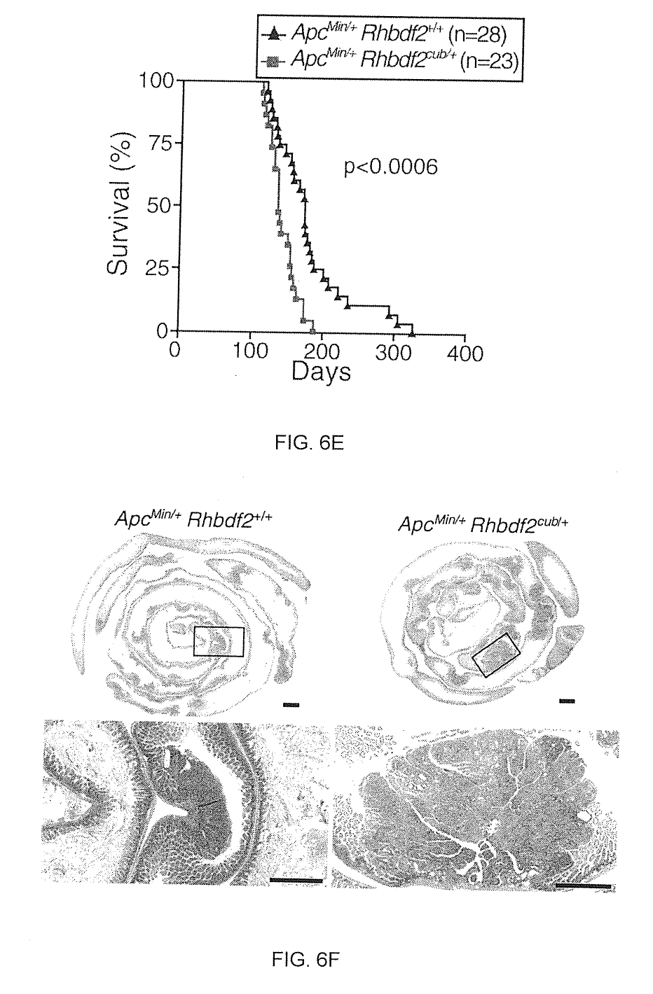

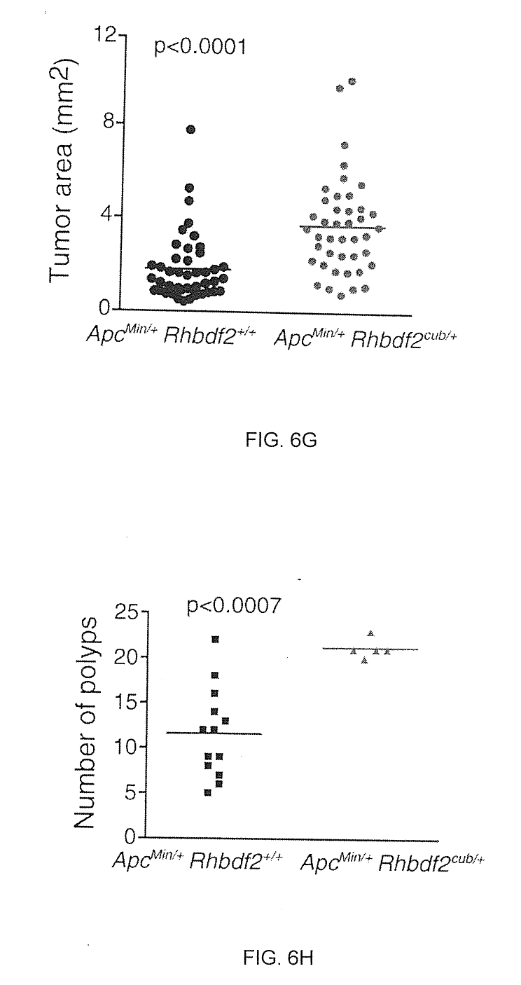

Missense mutations in RHBDF2 (p.I186T, p.P189L, and p.D188N) may underlie a familial tylosis with esophageal cancer syndrome (TOC). To test whether an increased AREG secretion due to dominant N-terminal mutations in RHBDF2 might drive these human pathologic changes in vitro by co-expressing AREG with a HuWt clone containing the human missense mutation (RHBDF2 p.I186T), HuCub, or HuWt in 293T cells. Expression of the RHBDF2 p.I186T or HuCub resulted in greater levels of AREG in conditioned medium and lower intracellular levels compared with HuWt (FIGS. 6A and 6B). Further, RHBDF2 p.I186T produced AREG levels comparable to those produced by HuCub, indicating that loss of, or dominant mutations in, the iRhom2 N-terminus lead to increased AREG secretion. Additionally, we found that loss of at least one of four critical residues (H, C, Q, and H) in the peptidase domain of the RHBDF2 p.I186T mutant resulted in significantly decreased AREG secretion (FIG. 6C).

To determine whether the Rhbdf2^{cub} allele increases tumor susceptibility, we investigated how its expression would affect adenoma formation in Apc^{Min/+} mice, a mouse model of human familial adenomatous polyposis. In Apc^{Min/+} mice, spontaneous loss of one wildtype Apc allele induces intestinal epithelial adenoma formation and premature death at a median age of 169 days. Notably, Rhbdf2 (FIG. 6D) and Areg expression is observed in the small intestine, suggesting a potential functional relationship. We generated and observed Apc^{Min/+} Rhbdf2^{+/cub} and Apc^{Min/+} Rhbdf2^{+/+} mice, but because of increased lethality we could not generate enough Apc^{Min/+} Rhbdf2^{cub/cub} mice for meaningful comparison. Nonetheless, we did observe a significant difference in the median survival age of Apc^{Min/+} Rhbdf2^{+/+} (172 days) and Apc^{Min/+} Rhbdf2^{+/cub} (135 days) mice (FIG. 6E). Necropsy of Apc^{Min/+} Rhbdf2^{+/+} and Apc^{Min/+} Rhbdf2^{+/cub} mice at 3 months of age revealed that the presence of a single Rhbdf2^{cub} allele significantly increased the number of polyps (FIGS. 6F and 6G) and adenoma size (FIGS. 6F and 6H) in Apc^{Min/+} mice, indicating that the cub mutation increases the growth of epithelial tumors. However, there was no spontaneous incidence of cancer in Rhbdf2^{cub/cub} mice aged up to two years, indicating that Rhbdf2^{cub} mutation creates a conducive environment for, but alone does not drive, tumor development.

### Example 7: Loss of the Cytosolic N-Terminus or Dominant Mutations in the N-Terminus of the RHBDF2 Gene Increase its Protein Stability

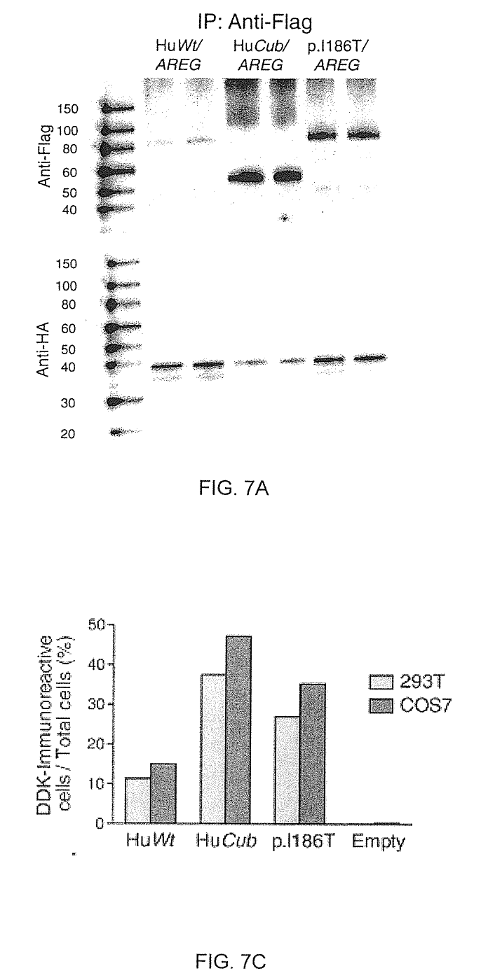

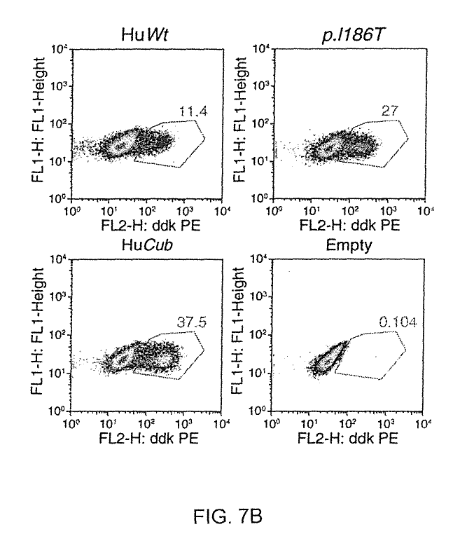

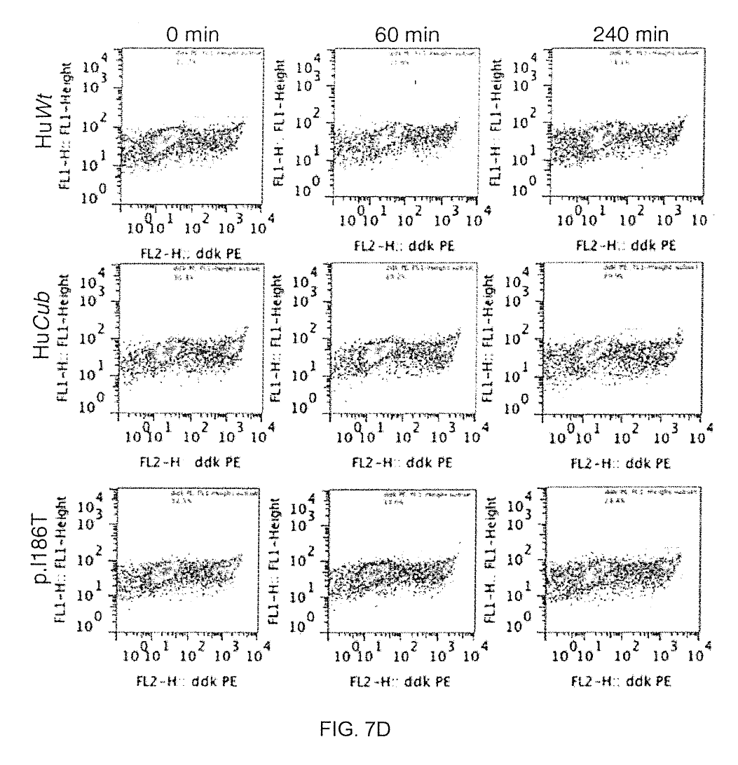

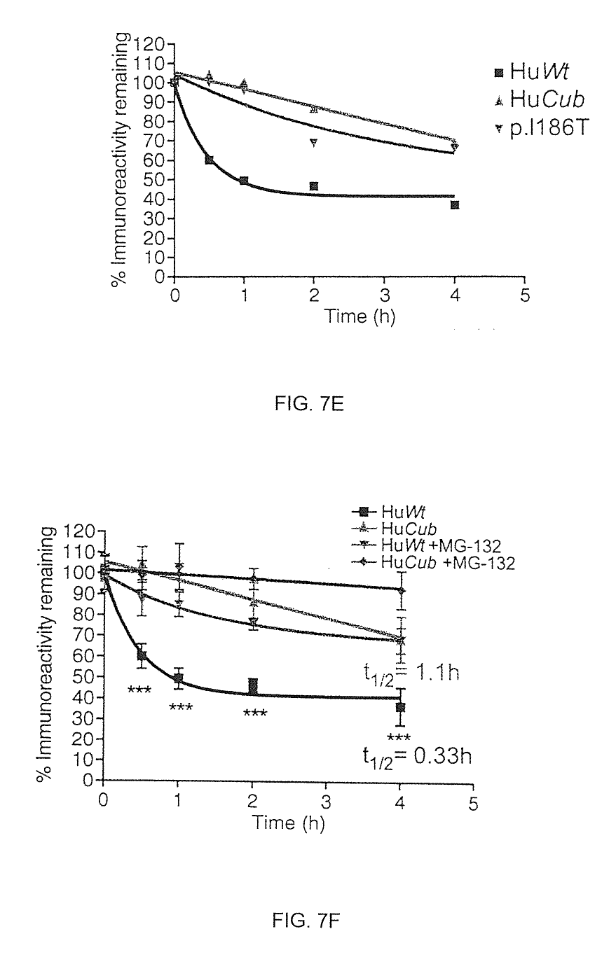

iRhom2 negatively regulates EGFR signaling by promoting degradation of EGF-like ligands through the proteasomal pathway. We determined that iRhoms induce secretion of AREG/HB-EGF when the cytosolic N-terminus is lacking (FIG. 5). To test whether gain-of-function mutations in the amino terminus of iRhom2 interfere with proteasomal processing and thereby increase its stability, we examined whether the tylotic RHBDF2 mutant p.I186T has an ability to interact with AREG. We found that, similar to HuWt and HuCub, p.I186T forms physical complexes with AREG (FIG. 7A). We then compared the protein expression levels of HuWt, HuCub and p.I186T in 293T and COS7 cells by immunocytochemistry and flow cytometry. We observed that HuWt protein expression was significantly lower compared to both HuCub and p.I186T (FIG. 7B,C). Further, when we subjected COS7 cells to a cyclohexamide (a protein synthesis inhibitor) chase for the indicated times, within one hour we observed an approximately 50% reduction in immunoreactivity for HuWt compared with either HuCub or the p.I186T (FIG. 7D,E).

To test whether iRhom2 is a target for proteasomal degradation, we determined the protein half-life of HuWt and HuCub in the presence of a potent proteasomal inhibitor MG-132. Expectedly, the protein half-life of HuWt, but not HuCub, was significantly increased (FIG. 7F), indicating that the proteasomal degradation of iRhom2 might be affecting its protein stability. We conclude that missense mutations in the amino terminus of iRhom2, similar to the Rhbdf2^{cub} mutation, increase its stability and contribute to enhanced AREG secretion independent of metalloprotease activity.

### Example 8

Here we report that iRhom2, a member of a family of rhomboid proteases well known as regulators of EGFR signaling in Drosophila, has an ability to regulate EGFR signaling during cutaneous healing and tumor development. We show that iRhom2 is a short-lived protein whose stability can be increased by select mutations in the N-terminal domain. In turn, these stable variants function to enhance AREG secretion independent of metalloprotease activity. We identify an important role for iRhoms in EGFR-dependent cell proliferation and wound healing and show how iRhom2 mutations that increase EGFR signaling, under the right circumstances, can promote cancer development.

The N-Terminal and the Peptidase Domains have Separate Functions in Regulating EGFR Signaling

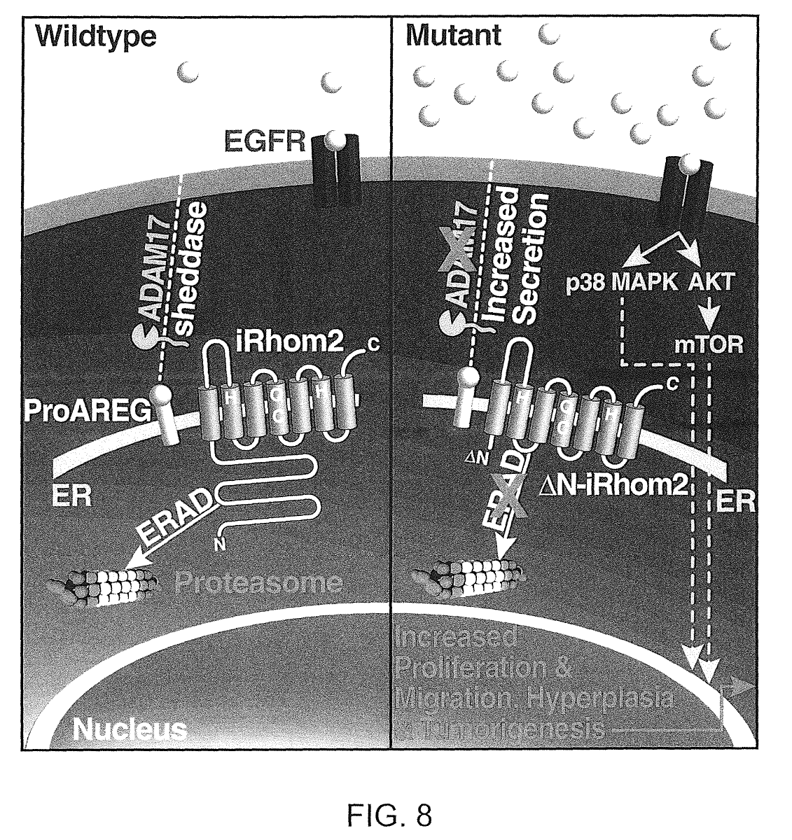

iRhoms are complex multi-domain enzymes that contain a long cytosolic N-terminus, a dormant peptidase domain and a conserved iRhom homology domain (IRHD); the function of these domains remains unknown. Under normal circumstances, iRhoms negatively regulate EGFR signaling by promoting the degradation of EGF-like substrates. However, the Rhbdf2^{cub} mutation is unlikely to be simply a loss-of-function mutation. Rhbdf2^{-/-} mice failed to recapitulate the Rhbdf2^{cub} phenotype. In addition, we demonstrate that, similar to the Rhbdf2^{cub} mutation, dominant missense mutations in the N-terminus of iRhom2 induce secretion of AREG and HB-EGF, in a manner mediated by key amino acids in transmembrane helices two, four, and six of the peptidase domain. Thus, our results suggest that the cytosolic N-terminus of iRhom2 negatively regulates EGFR signaling by suppressing the peptidase domain and, consequently, secretion of AREG/HB-EGF (FIG. 8). Our findings also reveal more subtle regulatory functions for iRhom2 that are unmasked in N-terminal mutations such as Rhbdf2^{cub}.

The Rhbdf2^{cub} may be considered a gain-of-function mutation. This conclusion is supported by several pieces of evidence. First, the negative regulatory role of iRhom2 seems to be minimal because Rhbdf2^{-/-} mice do not present an overt ‘EGFR hyperactive’ phenotype except when combined with the Rhbdf2^{cub} mutation. Second, co-transfection of HuCub and AREG results in approximately 2-3-fold greater levels of AREG compared with transfections of either HuWt and AREG or AREG alone. Third, expression of HuCub induced secretion of membrane-anchored AREG and HB-EGF independent of metalloprotease activity. Fourth, mutant iRhom2 alleles fail to induce secretion of AREG/HB-EGF in the absence of the peptidase domain. Consistent with these observations, transgenic expression of the N-terminal-truncated but not full-length RHBDF1 induces a strong EGFR signaling-related wing phenotype in Drosophila. Additionally, co-expression of truncated RHBDF1 with HB-EGF intensifies the altered wing phenotype in Drosophila, indicating that the truncated iRhom1 might induce secretion of HB-EGF and thereby activate EGFR signaling. These results validate the concept that the cytosolic N-terminus contributes to iRhom-elicited ubiquitin processing of EGF family ligands, while the iRhom peptidase domain stimulates EGFR signaling when not suppressed by the N-terminus.

We found that mutant iRhom2 alleles induce secretion of AREG and HB-EGF in the presence of saturating concentrations of marimastat, a potent, broad-spectrum metalloprotease inhibitor. Maturation of other members of the ADAM family is unaffected by the deficiency of both iRhoms. Moreover, we demonstrate that ADAM17 activity is attenuated in Rhbdf2^{cub} mice. Thus, we conclude that Rhbdf2^{cub} and tylotic mutations selectively induce secretion of AREG and HB-EGF independent of metalloprotease activity, and the transmembrane peptidase domain is necessary for this function.

A Hyperactive EGFR Pathway Underlies Accelerated Cutaneous Healing in Rhbdf2^{cub} Mice

The biology of wound healing is complex. In humans, adult wounds are vulnerable to non-functional fibrotic tissue formation, while prenatal wounds accomplish complete regeneration, resembling scar-free healing in vertebrates such as axolotls and planarians.

The tissue regeneration and remodeling field is still in its infancy. In vitro wound healing assays performed in a human keratinocyte cell line indicate that the expression of RHBDL2 is significantly upregulated after wounding compared with unwounded controls.

The ability of Rhbdf2^{cub} mice to rapidly heal wounds without significant scar formation might be due to a combination of decreased TNFα secretion due to attenuated ADAM17 activity and rapid re-epithelialization induced by augmented AREG production/EGFR hyperactivation. We propose that while decreased TNFα contributes to a lesser degree of inflammation, increased AREG production facilitates accelerated proliferation and migration of keratinocytes to the wound site in Rhbdf2^{cub} mice. Moreover, iRhom2 is predominantly expressed in the skin, making it a potential new therapeutic target in impaired cutaneous wound healing.

The iRhom2-AREG-EGFR Pathway is Constitutively Active in Some Epithelial Cancers

In the present study, we find that N-terminal-truncated iRhom2 promotes increased AREG production independent of ADAM17 activity and thereby induces EGFR activation. In particular, the phenotype of Apc^{Min/+} Rhbdf2^{+/cub} mice recapitulates the increased susceptibility to epithelial cancers seen in patients with dominant RHBDF2 mutations. However, Rhbdf2^{cub/cub} mice did not spontaneously develop tumors, indicating that the Rhbdf2^{cub} mutation might not drive cancer development but instead might promote tumor growth and progression by creating a conducive environment.

Materials and Methods

Mice were obtained, bred and maintained under modified barrier conditions at The Jackson Laboratory (Bar Harbor, Me.). All genotypes, including cub and Mcub, were maintained on the C57BL/6J (B6) genetic background. To generate Rhbdf2^{-/-} mice, ES cell clones (EPD0208_1_A09) obtained from the Knockout Mouse Program (KOMP) repository were injected into B6-Tyr^{c} (B6 albino) blastocysts. Males displaying >50% chimerism were mated to B6 albino females; black offspring were genotyped by PCR. Heterozygotes were mated with each other to produce homozygotes, or with Rhbdf2^{cub} mice to produce Rhbdf2^{-/cub} mice. The Animal Care and Use Committee at The Jackson Laboratory approved all the experimental procedures.

Cell culture and Reagents—We isolated MEFs as previously described. Mouse epidermal keratinocytes (MEKs) were isolated according the manufacturer's instructions (CELLnTEC/Zenbio, Research Triangle Park, NC). Cells were grown in a humidified chamber at 370 C with 5% CO2. MEF reagents [Phosphate buffered saline (PBS), HyClone Dulbeccos Modified Eagles Medium, High Glucose (DMEM), penicillin-streptomycin and HyClone fetal bovine serum) and MEK reagents (progenitor cell targeted medium CnT-07, antibiotic/antimycotic solution ABM, and protease Dispase) were purchased from Fisher Scientific (Boston, Mass.) and Zen-Bio (Research Triangle Park, NC), respectively. TrypLE™ Select was obtained from Life technologies (Chicago, Ill.). 293T and COS7 cells were grown in DMEM and RPMI medium, respectively.

Histology—Preparation of the tissues for histological examination was performed as previously described.

Ear hole closure: Mice were wounded using a surgical ear punch device Napox KN-292B to make 2 mm through-and-through holes in the center of the left ear, and the closure rates were analyzed over a period of 28 days. At the indicated times, mice were euthanized and images of the ear holes and surrounding tissue were captured using the 4× objective (Olympus BX41 microscope). ImageJ software analyzed the wound area. Ears were fixed in 10% neutral buffered formalin (NBF) for 24 h at room temperature and processed for paraffin embedding and sectioning. The physical wound area was measured at day 0 and measurements at all subsequent time points were calculated as a percent of the original area. Proliferation and scratch-wound assays: Both sets of assays were performed using MEFs. For the proliferation assays, the indicated numbers of cells were grown in collagen-coated 96-well plates at 37 C. After 24 h of incubation, the growth medium was removed and the plates were frozen at −80° C. until use. CyQUANT® Cell Proliferation kit was used to determine the relative cell numbers (Life technologies). Fluorescence intensity was measured using Victor³ multilabel plate reader (PerkinElmer, Boston, Mass.). For the scratch-wound assays, MEFs were grown to confluence in collagen-coated 6-well plates and a strip of cells was removed from the confluent monolayer by drawing a sterile p200 pipette tip across the well. Scratches were imaged at 0, 3, 6, 9 and 12 h using the 5× objective on a Zeiss Observer phase contrast microscope and Axio Vision 4.6.3 software. Using the GRID plugin we overlaid a grid on each image. The grid size was such that there were at least 10 lines bisecting the scratch image. The widths for each scratch were compiled and averaged. ImageJ software analyzed the captured images from the 0, 3, 6, 9 and 12 h time series.

Human AREG, HB-EGF and EGF ELISA: Transient transfections were performed in 293T cells seeded in 24-well plates. Using lipofectamine LTX with Plus reagent 16.5 fmol of HuWt or HuCub was co-transfected with 25 ng of AREG or 50 ng of HB-EGF or 200 ng of EGF. The total amount of transfected DNA was made up to equal amount per well with pCMV6-AC-HA vector. After 24 h, DMSO or 10 μM MM in 1 ml of fresh medium replaced the old medium. After 24 h, 100 μl of supernatant was either undiluted (HB-EGF and EGF) or diluted five-fold (AREG) and subjected to ELISA as per the manufacturer's instructions (DY262, DY259, and DY236).

Expression plasmids containing cDNA encoding human AREG (RC203150), human HB-EGF (SC108485), human EGF (SC127840), human RHBDF2 (RC203923), mouse Rhbdf1 (MC205414), and human RHBDL2 (RC219882) were obtained from OriGene (Rockville, Md.). Mutations (deletions or insertions) in the full-length RHBDF2 or mouse Rhbdf1 were introduced using the QuikChange II XL mutagenesis kit (Agilent Technologies, Santa Clara, Calif.) according to the manufacturer's instructions and verified by DNA sequencing. Primers for site-directed mutagenesis were generated using the Primer Design Program (QuikChange® Primer Design, Agilent Technologies).

Immunochemistry: MEFs plated in BD BioCoat™ Collagen I 2-well culture slides were grown to ˜60% confluence and transiently transfected with Lipofectamine™ LTX (Life technologies) according to the manufacturer's instructions. After 48 h, MEFs were fixed in acetone and subjected to immunocytochemistry. Briefly, cells washed in 1× ice-cold PBS were fixed in acetone at −20° C. for 10 min followed by three additional washes in PBS. Endogenous peroxidase activity was blocked with 3% H2O2, followed by incubations in blocking buffer (PBST, 10% goat serum and 1% BSA) for 1 h and DDK-specific antibody (Origene) at 4° C. overnight. The following day, cells were washed in PBST for 25 min and incubated in HRP-conjugated secondary antibody for 30 min at room temperature. After three washes in PBST, cells were incubated in DAPI solution (IHC World, Woodstock, Md.) for 10 min before cover slipping and sealing the slides. Calnexin and giatin antibodies were obtained from Abcam (Cambridge, Mass.). Secondary antibodies were obtained from Life technologies.

Immunoprecipitation: 293T and COS7 cell transfections were carried according to the manufacturer's instructions (Life technologies). Cells plated in 6 well plates were treated with 400 μls of Cell Lysis Buffer (Cell Signaling, Danvers, Mass.) containing complete mini protease inhibitors (Roche Applied Science, Indianapolis, Ind.), incubated on ice for 10 min and centrifuged at 14,000 g for 10 min in a cold room. Cell supernatant was incubated overnight with 20 μls of magnetic beads conjugated with Anti-DDK antibody (Origene) in a cold room on a rocking platform. Following overnight incubation, beads/slurry was washed with three times with TBS-T, and protein was collected using 2×SDS-PAGE loading buffer.

SDS-PAGE and Immunoblotting: Cells were washed once with ice cold PBS and incubated with RIPA buffer (Cell Signaling, Danvers, Mass.) containing complete mini protease inhibitors (Roche Applied Science, Indianapolis, Ind.) on ice for 10 min. Cell lysate was centrifuged at 14,000 g for 10 min at 4° C. Supernatant was collected and stored at −80° C. until further use. Total amount of protein was quantified using a Qubit Fluorometer (Life technologies), and 10 μg of protein was loaded onto 4-12% pre-cast gels (Lonza, Allendale, N.J.) and run at 140V until the low molecular weight Precision plus protein standard (Bio-Rad) reached the bottom of the gel. Resolved proteins were electrophoretically transferred onto PVDF membranes using an iBLOT (Life technologies) before blocking in 5% BSA (Cell Signaling Technology) in TBST (20 mM Tris, 137 mM sodium chloride, and 0.1% Tween-20) for 1 h at room temperature. Membranes were then incubated overnight at 4° C. in primary antibodies (1:1000) followed by three 15 min washes in TBST and incubation in secondary antibodies (1:2000) for 1 h at room temperature. After another wash in TBST for 90 min, membranes were exposed to SuperSignal West Femto Substrate (Fisher Scientific) for 5 min for detection of horseradish peroxidase generated signal. Membranes were re-probed with actin-specific antibody. All the antibodies were purchased from Cell Signaling Technology (Danvers, Mass.). DDK-antibody was obtained from Origene (Rockville, Md.).

Amphiregulin silencing in primary MEFs: Murine Areg shRNA clone TRCN0000089050 (in lentiviral vector pLKO.1) and scramble control shRNA in pLKO.1 (plasmid #1864) were obtained from Sigma Aldrich (St. Louis, Mo.) and Addgene (Cambridge, Mass.), respectively. Lentiviral particles were produced in 293T/17 cells (ATCC) using plasmids pMDLg/pRRE, pRSV-rev, and pMD2.g (Addgene). Lentiviral supernatants were collected at 48 h and 60 h, combined, and filtered through 0.45 μm PVDF filters. MEFs were infected twice on successive days with lentiviral supernatants in the presence of 5 μg/ml polybrene, followed by selection with puromycin (2 μg/ml) for 72 hours prior to being used in downstream experiments.

Polyp counts and tumor area—Intestinal tracts removed from 12-week-old euthanized mice were flushed and fixed in 10% neutral buffered formalin for at least 24 h and subjected to polyp counting or histological examination. H&E stained slides were scanned using a digital scanner NanoZoomer 2.0HT (Hamamatsu, Japan). Polyps were either counted directly under a dissecting microscope or from the scanned digital images. Notably, there were no significant differences in polyp counts between the two methods. Tumor area was also measured from the scanned digital images using the NanoZoomer 2.0HT software.

Quantitative real-time PCR (qPCR)—Purified RNA from skin was isolated as per the manufacturer's instructions using the TRIzol® Plus RNA purification kit (Life Technologies, Chicago, Ill.). The Agilent 2100 Bioanalyzer determined the quality of purified RNA. qPCR cycling conditions have been published previously. We obtained the following list of predesigned TaqMan gene expression assays from Life Technologies (Chicago, Ill.), Table I.

TABLE I

| Gene | Assay | Spanning Exons |
|---|---|---|
| Rhbdf2 | Mm00553469_m1 | 2-3 |
| Rhbdf2 | Mm00553471_m1 | 4-5 |
| Rhbdf2 | Mm01248820_g1 | 7-8 |
| Rhbdf2 | Mm00553476_m1 | 9-10 |
| Rhbdf2 | Mm00553479_g1 | 12-13 |
| Rhbdf2 | Mm01248816_g1 | 14-15 |
| Rhbdf2 | Mm01248818_g1 | 17-18 |
| Areg | Mm00437583_m1 | — |
| Hbegf | Mm00439307_m1 | — |
| Ereg | Mm00514794_m1 | — |
| Epgn | Mm00504344_m1 | — |
| Tgfa | Mm00446232_m1 | — |
| Egf | Mm00438696_m1 | — |
| Btc | Mm00432137_m1 | — |
| Actb | 4352933E (Part #) | — |

Mouse TNFα and AREG ELISA—For mouse TNFα ELISA, 8-12 week-old female mice of the indicated genotype were injected intravenously with 70 μg of Lipopolysaccharide from E. coli O111:B4 (Sigma-Aldrich, St. Louis, Mo.). Serum was collected after 3 h and frozen at −80° C. For AREG ELISA, serum collected from all genotypes was used immediately or stored not more than 24 h at −20° C. Samples were assayed using DuoSet ELISA Developmental kits (R&D Systems, Minneapolis, Minn.) as per the manufacturer's instructions (DY989 and DY410).

Flow cytometry—293T and COS7 cells plated in 6 well plates were transiently transfected with 600 ng of HuWt or HuCub or HuWt p.I186T cDNAs using Lipofectamine™ LTX reagent. After 40 h, cells were incubated in 150 μg/ml of cyclohexamide for 0, 0.5, 1, 2 and 4 h. Following that, cells were fixed in 150 μls of freshly prepared cold 4% PFA at RT for 40 min. Cells were washed once with 2 ml PBST and resuspended in 150 μls of PBST for 15 min at RT in the dark. After another wash, cells were resuspended in 125 μls of PBST containing phycoerythrin-labeled anti-DDK primary antibody (1:50) on ice for 30 min. After a final wash, cells were resuspended in FACS buffer and subjected to flow cytometry analysis (BD FACSCalibur).

Reverse transcriptase PCR (RT-PCR)—RNA was extracted from MEFs using RNAqueous™-4PCR (Ambion) according to the manufacturer's protocol. cDNA was synthesized from 1 μg of RNA using reverse transcriptase (Promega reverse transcription systems, Promega). cDNA was amplified using specific primers with HotMaster TAQ (5-Prime Inc.).

Statistical analysis—GraphPad Prism v4 (GraphPad Software, San Diego, Calif.) was used to generate Kaplan-Meier survival curves and calculate protein half-life (One phase exponential decay equation). One-way ANOVA and t-tests determined statistical differences. P value less than 0.05 was considered statistically significant.

Items

1. A method for identifying a compound that activates the proteolytic activity of an iRhom polypeptide on a substrate comprising:

a) contacting a cell expressing an iRhom polypeptide with a test compound; and

b) determining an increase in stability of the iRhom polypeptide relative to an appropriate control, wherein an increase in stability of the iRhom polypeptide indicates that the compound activates the proteolytic activity of the iRhom polypeptide on a substrate.

2. The method of item 1, wherein an increase in stability of the iRhom polypeptide is determined by analyzing the half-life of the iRhom polypeptide following exposure to the compound.

3. The method of item 1, wherein the substrate is an EGFR ligand or an EGF-like substrate.

4. The method of item 1, wherein an increase in stability of the iRhom polypeptide is determined by detecting an increase in secretion of an EGFR ligand.

5. The method of item 4, wherein the EGFR ligand is selected from the group consisting of AREG, HB-EGF, TGFα and EPGN.

6. The method of item 1, wherein an increase in stability of an iRhom polypeptide is determined by detecting an increase in the level of soluble EGFR ligands.

7. The method of item 6, wherein the EGFR ligand is selected from the group consisting of AREG, HB-EGF, TGFα and EPGN.

8. The method of item 1, wherein an increase in stability of an iRhom polypeptide is determined by detecting an increase in EGFR signaling activity.

9. The method of item 1, wherein the compound inhibits an interaction between the iRhom polypeptide and a proteasome.

10. The method of item 1, wherein the compound inactivates the cytoplasmic domain of an iRhom polypeptide.

11. The method of item 10, wherein the inactivation of the cytoplasmic domain is transient.

12. The method of item 1, wherein the compound cleaves and/or deletes the cytoplasmic domain of an iRhom polypeptide such that the polypeptide has proteolytic activity or altered biological activity.

13. The method of item 12, wherein mouse iRhom2 is cleaved between amino acid residues 1 and 268 or human iRhom2 is cleaved between amino acid residues 1 and 298.

14. The method of item 12, wherein mouse iRhom1 is cleaved between amino acid residues 1 and 272 or human iRhom1 is cleaved between amino acid residues 1 and 316.

15. The method of item 1, wherein the compound activates the peptidase domain of an iRhom polypeptide.

16. The method of any one of item 1-15, wherein the iRhom polypeptide is iRhom1 or iRhom2.

17. The method of item 15, wherein the iRhom polypeptide is a human or mouse iRhom polypeptide.

18. The method of item 1, wherein the compound is selected from the group consisting of a small molecule, a peptide or a polypeptide decoy.

19. The method of item 18, wherein the compound is attached to a cell penetrating peptide.

20. The method of item 1, wherein the compound accelerates migration of keratinocytes.

21. The method of item 1, wherein the compound accelerates proliferation of fibroblasts.

22. The method of item 1, wherein the compound is cell-permeable.

23. The method of item 1, wherein the compound is an ubiquitin protease inhibitor.

24. A method for identifying a compound that inhibits the proteolytic activity of an iRhom polypeptide on a substrate comprising:

a) contacting a cell expressing an iRhom polypeptide with a test compound; and

b) determining a decrease in secretion of a physiological target of the iRhom polypeptide relative to an appropriate control or a decrease in EGFR activity relative to an appropriate control, wherein a decrease in secretion of a physiological target of the iRhom polypeptide or a decrease in EGFR activity indicates that the compound inhibits the proteolytic activity of the iRhom polypeptide on a substrate.

25. The method of item 24, wherein the substrate is an EGFR ligand or an EGF-like substrate.

26. The method of item 24, wherein the inhibition of proteolytic activity of the iRhom polypeptide is determined by detecting a decrease in secretion of a physiological target of the iRhom polypeptide.

27. The method of item 26, wherein the physiological target of the iRhom polypeptide is an EGFR ligand.

28. The method of item 27, wherein the EGFR ligand is selected from the group consisting of AREG, HB-EGF, TGFα and EPGN.

29. The method of item 24, wherein the inhibition of proteolytic activity of the iRhom polypeptide is determined by detecting a decrease in the level of soluble EGFR ligands.

30. The method of item 29, wherein the EGFR ligand is selected from the group consisting of AREG, HB-EGF, TGFα and EPGN.

31. The method of item 24, wherein the inhibition of proteolytic activity of an iRhom polypeptide is determined by detecting a decrease in EGFR activity.

32. The method of item 24, wherein the compound inhibits the peptidase domain of an iRhom polypeptide.

33. The method of item 24, wherein the compound affects the activity of an iRhom polypeptide by inactivating one or more amino acid residues in the proteolyic site of the iRhom polypeptide peptidase domain.

34. The method of item 33, wherein the iRhom polypeptide is mouse iRhom2 and the compound inactivates one or more amino acid residues in the proteolytic site of mouse iRhom2 selected from the group consisting of Histidine 635 on TM helix 2, Glutamine 695 on TM helix 4, Cysteine 701 on transmembrane helix 4 and Histidine 744 on TM helix 6.

35. The method of item 33, wherein the iRhom polypeptide is human iRhom2 and the compound inactivates one or more amino acid residues in the proteolytic site of human iRhom2 selected from the group consisting of Histidine 664 on TM helix 2, Glutamine 724 on TM helix 4, Cysteine 730 on transmembrane helix 4 and Histidine 773 on TM helix 6.

36. The method of any one of items 24-33, wherein the iRhom family member is iRhom1 or iRhom2.

37. The method of item 33, wherein the iRhom family member is a human or mouse iRhom family member.

38. The method of item 24, wherein the compound is selected from the group consisting of a small molecule, a peptide, or a polypeptide decoy.

39. The method of item 38, wherein the compound is attached to a cell penetrating peptide.

40. The method of item 24, wherein the compound is cell-permeable.

41. A method for identifying a compound capable of accelerating wound healing or tissue repair in a subject comprising: a) contacting a cell expressing an iRhom polypeptide and an EGFR ligand with a test compound; b) determining an increase in stability of the iRhom polypeptide relative to an appropriate control; and c)
determining an increase in secretion of an EGFR ligand by the cell relative to an appropriate control, wherein an increase in stability of the iRhom polypeptide and an increase in secretion of an EGFR ligand by the cell indicates that the compound is capable of accelerating wound healing.

42. The method of item 41, wherein the iRhom family member is iRhom1 or iRhom2.

43. The method of item 41, wherein the iRhom family member is a human or mouse iRhom family member.

44. A method for identifying a compound capable of reducing tumor growth and/or progression or treating cancer in a subject comprising: a) contacting a cell expressing an iRhom polypeptide with a test compound; and b) determining a decrease in secretion of a physiological target of the iRhom polypeptide relative to an appropriate control or a decrease in EGFR activity relative to an appropriate control, wherein a decrease in secretion of a physiological target of the iRhom polypeptide or a decrease in EGFR activity indicates that the compound is capable of reducing tumor growth and/or progression or treating cancer in the subject.

45. The method of item 44, wherein the tumor is a solid tumor.

46. The method of item 44, wherein the cancer is an epithelial cancer.

47. The method of item 44, wherein the cancer is cancer of the esophagus, lung, brain, colon, kidney, prostate, skin, liver, pancreas, stomach, uterus, ovary, breast, lymph glands or bladder.

48. A method for identifying a compound that inhibits the cytoplasmic domain of an iRhom polypeptide comprising: a) contacting a cell expressing an iRhom polypeptide with a test compound; b) determining an increase in stability of the iRhom polypeptide relative to an appropriate control, wherein an increase in stability of the iRhom polypeptide indicates that the compound inhibited the cytoplasmic domain of the iRhom polypeptide.

49. The method of any one of items 44-48, wherein the iRhom family member is iRhom1 or iRhom2.

50. The method of any one of items 44-48, wherein the iRhom family member is a human or mouse iRhom family member.

51. The method of item 41, wherein the compound is selected from the group consisting of a small molecule, a polypeptide decoy, an miRNA molecule, an siRNA molecule, an shRNA molecule, a dsRNA molecule, an antisense molecule, a ribozyme specific for Rhbdf2; or a polynucleotide encoding the miRNA, siRNA, shRNA, dsRNA; or a biological equivalent of each thereof.

52. An isolated polypeptide comprising a variant iRhom polypeptide.

53. An isolated polypeptide comprising human iRhom2 with a deletion of the cytosolic N-terminal domain.

54. An isolated polypeptide comprising mouse iRhom2 with a deletion of the cytosolic N-terminal domain.

55. An isolated mouse iRhom2 polypeptide comprising a mutation at one or more amino acid residues in the proteolytic site of mouse iRhom2 selected from the group consisting of Histidine 635 on TM helix 2, Glutamine 695 on TM helix 4, Cysteine 701 on TM helix 4 and Histidine 744 on TM helix 6.

56. An isolated human iRhom2 polypeptide comprising a mutation at one or more amino acid residues in the proteolytic site of human iRhom2 selected from the group consisting of Histidine 664 on TM helix 2, Glutamine 724 on TM helix 4, Cysteine 730 on TM helix 4 and Histidine 773 on TM helix 6.

57. An isolated nucleic acid molecule encoding the polypeptide of any one of claims 52-56.

58. A vector comprising the nucleic acid molecule of claim 57.

59. A host cell expressing the vector of claim 58.

60. A method for identifying a compound capable of promoting hair growth in a subject comprising: a) contacting a cell expressing an iRhom polypeptide with a test compound; and b) determining a decrease in secretion of a physiological target of the iRhom polypeptide relative to an appropriate control or a decrease in EGFR activity relative to an appropriate control, wherein a decrease in secretion of a physiological target of the iRhom polypeptide or a decrease in EGFR activity indicates that the compound is capable of promoting hair growth in the subject.

SEQUENCES

Mouse iRhom2 protein sequence full-length SEQ ID
NO: 1
MASADKNGSNLPSVSGSRLQSRKPPNLSITIPPPESQAPGEQDSMLPERR
KNPAYLKSVSLQEPRGRWQEGAEKRPGFRRQASLSQSIRKSTAQWFGVSG
DWEGKRQNWHRRSLHHCSVHYGRLKASCQRELELPSQEVPSFQGTESPKP
CKMPKIVDPLARGRAFRHPDEVDRPHAAHPPLTPGVLSLTSFTSVRSGYS
HLPRRKRISVAHMSFQAAAALLKGRSVLDATGQRCRHVKRSFAYPSFLEE
DAVDGADTFDSSFFSKEEMSSMPDDVFESPPLSASYFRGVPHSASPVSPD
GVHIPLKEYSGGRALGPGTQRGKRIASKVKHFAFDRKKRHYGLGVVGNWL
NRSYRRSISSTVQRQLESFDSHRPYFTYWLTFVHIIITLLVICTYGIAPV
GFAQHVTTQLVLKNRGVYESVKYIQQENFWIGPSSIDLIHLGAKFSPCIR
KDQQIEQLVRRERDIERTSGCCVQNDRSGCIQTLKKDCSETLATFVKWQN
DTGPSDKSDLSQKQPSAVVCHQDPRTCEEPASSGAHIWPDDITKWPICTE
QAQSNHTGLLHIDCKIKGRPCCIGTKGSCEITTREYCEFMHGYFHEDATL
CSQVHCLDKVCGLLPFLNPEVPDQFYRIWLSLFLHAGIVHCLVSVVFQMT
ILRDLEKLAGWHRISIIFILSGITGNLASAIFLPYRAEVGPAGSQFGLLA
CLFVELFQSWQLLERPWKAFFNLSAIVLFLFICGLLPWIDNIAHIFGFLS
GMLLAFAFLPYITFGTSDKYRKRALILVSLLVFAGLFASLVLWLYIYPIN
WPWIEYLTCFPFTSRFCEKYELDQVLH
Rhbdf2^{cub} mouse mutation SEQ ID NO: 2
MSSMPDDVFESPPLSASYFRGVPHSASPVSPDGVHIPLKEYSGGRALGPG
TQRGKRIASKVKHFAFDRKKRHYGLGVVGNWLNRSYRRSISSTVQRQLES
FDSHRPYFTYWLTFVHIIITLLVICTYGIAPVGFAQHVTTQLVLKNRGVY
ESVKYIQQENFWIGPSSIDLIHLGAKFSPCIRKDQQIEQLVRRERDIERT
SGCCVQNDRSGC1QTLKKDCSETLATFVKWQNDTGPSDKSDLSQKQPSAV
VCHQDPRTCEEPASSGAHIWPDDITKWPICTEQAQSNHTGLLHIDCKIKG
RPCCIGTKGSCEITTREYCEFMHGYFHEDATLCSQVHCLDKVCGLLPFLN
PEVPDQFYRIWLSLFLHAGIVHCLVSVVFQMTILRDLEKLAGWHRISIIF
ILSGITGNLASAIFLPYRAEVGPAGSQFGLLACLFVELFQSWQLLERPWK
AFFNLSAIVLFLFICGLLPWIDNIAHIFGFLSGMLLAFAFLPYITFGTSD
KYRKRALILVSLLVFAGLFASLVLWLYIYPINWPWIEYLTCFPFTSRFCE
KYELDQVLH
Mouse iRhom1 protein sequence full-length SEQ ID
NO: 3
MSEARRDSTSSLQRKKPPWLKLDIPAAVPPAAEEPSFLQPLRRQAFLRSV
SMPAETARVPSPHHEPRRLVLQRQTSITQTIRRGTADWFGVSKDSDSTQK
WQRKSIRHCSQRYGKLKPQVIRELDLPSQDNVSLTSTETPPPLYVGPCQL
GMQKIIDPLARGRAFRMADDTADGLSAPHTPVTPGAASLCSFSSSRSGFN
RLPRRRKRESVAKMSFRAAAALVKGRSIRDGTLRRGQRRSFTPASFLEED
MVDFPDELDTSFFAREGVLHEEMSTYPDEVFESPSEAALKDWEKAPDQAD
LTGGALDRSELERSHLMLPLERGWRKQKEGGPLAPQPKVRLRQEVVSAAG
PRRGQRIAVPVRKLFAREKRPYGLGMVGRLTNRTYRKRIDSYVKRQIEDM
DDHRPFFTYWLTFVHSLVTILAVCIYGIAPVGFSQHETVDSVLRKRGVYE
NVKYVQQENFWIGPSSEALIHLGAKFSPCMRQDPQVHSFILAAREREKHS
ACCVRNDRSGCVQTSKEECSSTLAVWVKWPVHPSAPDLAGNKRQFGSVCH
QDPRVCDEPSSEDPHEWPEDITKWPICTKSSAGNHTNHPHMDCVITGRPC
CIGTKGRCEITSREYCDFMRGYFHEEATLCSQVHCMDDVCGLLPFLNPEV
PDQFYRLWLSLFLHAGILHCLVSVCFQMTVLRDLEKLAGWHRIAIIYLLS
GITGNLASAIFLPYRAEVGPAGSQFGILACLFVELFQSWQILARPWRAFF
KLLAVVLFLFAFGLLPWIDNFAHISGFVSGLFLSFAFLPYISFGKFDLYR
KRCQIIIFQVVFLGLLAGLVVLFYFYPVRCEWCEFLTCIPFTDKFCEKYE
LDAQLH
Mouse iRhom1 N-terminal truncated protein sequence
(equivalent to Rhbdf2^{cub} mutation- No cytosolic
domain but retains transmembrane peptidase domain)
SEQ ID NO: 4
MSTYPDEVFESPSEAALKDWEKAPDQADLTGGALDRSELERSHLMLPLER
GWRKQKEGGPLAPQPKVRLRQEVVSAAGPRRGQRIAVPVRKLFAREKRPY
GLGMVGRLTNRTYRKRIDSYVKRQIEDMDDHRPFFTYWLTFVHSLVTILA
VCIYGIAPVGFSQHETVDSVLRKRGVYENVKYVQQENFWIGPSSEALIHL
GAKFSPCMRQDPQVHSFILAAREREKHSACCVRNDRSGCVQTSKEECSST
LAVWVKWPVHPSAPDLAGNKRQFGSVCHQDPRVCDEPSSEDPHEWPEDIT
KWPICTKSSAGNHTNHPHMDCVITGRPCCIGTKGRCEITSREYCDFMRGY
FHEEATLCSQVHCMDDVCGLLPFLNPEVPDQFYRLWLSLFLHAGILHCLV
SVCFQMTVLRDLEKLAGWHRIAIIYLLSGITGNLASAIFLPYRAEVGPAG
SQFGILACLFVELFQSWQILARPWRAFFKLLAVVLFLFAFGLLPWIDNFA
HISGFVSGLFLSFAFLPYISFGKFDLYRKRCQIIIFQVVFLGLLAGLVVL
FYFYPVRCEWCEFLTCIPFTDKFCEKYELDAQLH
Human iRhom2 protein sequence full-length SEQ ID
NO: 5
MASADKNGGSVSSVSSSRLQSRKPPNLSITIPPPEKETQAPGEQDSMLPE
GFQNRRLKKSQPRTWAAHTTACPPSFLPKRKNPAYLKSVSLQEPRSRWQE
SSEKRPGFRRQASLSQSIRKGAAQWFGVSGDWEGQRQQWQRRSLHHCSMR
YGRLKASCQRDLELPSQEAPSFQGTESPKPCKMPKIVDPLARGRAFRHPE
EMDRPHAPHPPLTPGVLSLTSFTSVRSGYSHLPRRKRMSVAHMSLQAAAA
LLKGRSVLDATGQRCRVVKRSFAFPSFLEEDVVDGADTFDSSFFSKEEMS
SMPDDVFESPPLSASYFRGIPHSASPVSPDGVQIPLKEYGRAPVPGPRRG
KRIASKVKHFAFDRKKRHYGLGVVGNWLNRSYRRSISSTVQRQLESFDSH
RPYFTYWLTFVHVIITLLVICTYGIAPVGFAQHVTTQLVLRNKGVYESVK
YIQQENFWVGPSSIDLIHLGAKFSPCIRKDGQIEQLVLRERDLERDSGCC
VQNDHSGCIQTQRKDCSETLATFVKWQDDTGPPMDKSDLGQKRTSGAVCH
QDPRTCEEPASSGAHIWPDDITKWPICTEQARSNHTGFLHMDCEIKGRPC
CIGTKGSCEITTREYCEFMHGYFHEEATLCSQVHCLDKVCGLLPFLNPEV
PDQFYRLWLSLFLHAGVVHCLVSVVFQMTILRDLEKLAGWHRIAIIFILS
GITGNLASAIFLPYRAEVGPAGSQFGLLACLFVELFQSWPLLERPWKAFL
NLSAIVLFLFICGLLPWIDNIAHIFGFLSGLLLAFAFLPYITFGTSDKYR
KRALILVSLLAFAGLFAALVLWLYIYPINWPWIEHLTCFPFTSRFCEKYE
LDQVLH
Human truncated iRhom2 (equivalent to Rhbdf2^{cub}
mutation- No cytosolic domain but retains trans-
membrane peptidase domain) SEQ ID NO: 6
MSSMPDDVFESPPLSASYFRGIPHSASPVSPDGVQIPLKEYGRAPVPGPR
RGKRIASKVKHFAFDRKKRHYGLGVVGNWLNRSYRRSISSTVQRQLESFD
SHRPYFTYWLTFVHVIITLLVICTYGIAPVGFAQHVTTQLVLRNKGVYES
VKYIQQENFWVGPSSIDLIHLGAKFSPCIRKDGQIEQLVLRERDLERDSG
CCVQNDHSGCIQTQRKDCSETLATFVKWQDDTGPPMDKSDLGQKRTSGAV
CHQDPRTCEEPASSGAHIWPDDITKWPICTEQARSNHTGFLHMDCEIKGR
PCCIGTKGSCEITTREYCEFMHGYFHEEATLCSQVHCLDKVCGLLPFLNP
EVPDQFYRLWLSLFLHAGVVHCLVSVVFQMTILRDLEKLAGWHRIAIIFI
LSGITGNLASAIFLPYRAEVGPAGSQFGLLACLFVELFQSWPLLERPWKA
FLNLSAIVLFLFICGLLPWIDNIAHIFGFLSGLLLAFAFLPYITFGTSDK
YRKRALILVSLLAFAGLFAALVLWLYIYPINWPWIEHLTCFPFTSRFCEK
YELDQVLH
Human iRhom1 protein sequence full-length SEQ ID
NO: 7
MSEARRDSTSSLQRKKPPWLKLDIPSAVPLTAEEPSFLQPLRRQAFLRSV
SMPAETAHISSPHHELRRPVLQRQTSITQTIRRGTADWFGVSKDSDSTQK
WQRKSIRHCSQRYGKLKPQVLRELDLPSQDNVSLTSTETPPPLYVGPCQL
GMQKIIDPLARGRAFRVADDTAEGLSAPHTPVTPGAASLCSFSSSRSGFH
RLPRRRKRESVAKMSFRAAAALMKGRSVRDGTFRRAQRRSFTPASFLEED
TTDFPDELDTSFFAREGILHEELSTYPDEVFESPSEAALKDWEKAPEQAD
LTGGALDRSELERSHLMLPLERGWRKQKEGAAAPQPKVRLRQEVVSTAGP
RRGQRIAVPVRKLFAREKRPYGLGMVGRLTNRTYRKRIDSFVKRQIEDMD
DHRPFFTYWLTFVHSLVTILAVCIYGIAPVGFSQHETVDSVLRNRGVYEN
VKYVQQENFWIGPSSEALIHLGAKFSPCMRQDPQVHSFIRSAREREKHSA
CCVRNDRSGCVQTSEEECSSTLAVWVKWPIHPSAPELAGHKRQFGSVCHQ
DPRVCDEPSSEDPHEWPEDITKWPICTKNSAGNHTNHPHMDCVITGRPCC
IGTKGRCEITSREYCDFMRGYFHEEATLCSQVHCMDDVCGLLPFLNPEVP
DQFYRLWLSLFLHAGILHCLVSICFQMTVLRDLEKLAGWHRIAIIYLLSG
VTGNLASAIFLPYRAEVGPAGSQFGILACLFVELFQSWQILARPWRAFFK
LLAVVLFLFTFGLLPWIDNFAHISGFISGLFLSFAFLPYISFGKFDLYRK
RCQIIIFQVVFLGLLAGLVVLFYVYPVRCEWCEFLTCIPFTDKFCEKYEL
DAQLH
Human iRhom1 N-terminal truncated protein sequence
(equivalent to Rhbdf2^{cub} Mutation- No cytosolic
domain but retains transmembrane peptidase domain)
SEQ ID NO: 8
MLPLERGWRKQKEGAAAPQPKVRLRQEVVSTAGPRRGQRIAVPVRKLFAR
EKRPYGLGMVGRLTNRTYRKRIDSFVKRQIEDMDDHRPFFTYWLTFVHSL
VTILAVCIYGIAPVGFSQHETVDSVLRNRGVYENVKYVQQENFWIGPSSE
ALIHLGAKFSPCMRQDPQVHSFIRSAREREKHSACCVRNDRSGCVQTSEE
ECSSTLAVWVKWPIHPSAPELAGHKRQFGSVCHQDPRVCDEPSSEDPHEW
PEDITKWPICTKNSAGNHTNHPHMDCVITGRPCCIGTKGRCEITSREYCD
FMRGYFHEEATLCSQVHCMDDVCGLLPFLNPEVPDQFYRLWLSLFLHAGI
LHCLVSICFQMTVLRDLEKLAGWHRIAIIYLLSGVTGNLASAIFLPYRAE
VGPAGSQFGILACLFVELFQSWQILARPWRAFFKLLAVVLFLFTFGLLPW
IDNFAHISGFISGLFLSFAFLPYISFGKFDLYRKRCQIIIFQVVFLGLLA
GLVVLFYVYPVRCEWCEFLTCIPFTDKFCEKYELDAQLH

TABLE II
Primers used to amplify exons of mcub candidate genes Epgn, Ereg, Areg,
and Btc to compare V/Le strain DNA sequence with other strains. The Mcub allele
originated from the V/Le strain

| | | |
|---|---|---|
| | | Product |
| Primer sequence | Exon | size |

Genomic sequence of exons and flanking sequence

| | | | | | |
|---|---|---|---|---|---|
| Forward | Epgn1F | AAAACCCTCCACCC | Exon1 | 182 | SEQ ID NO: 9 |
| | | TTCC | | | |
| Reverse | Epgn1R | CGGGGAACAACTCT | | | SEQ ID |
| | | GAATCC | | | NO: 10 |
| Forward | Epgn2F | AGAGCCACTTTGGG | Exon2 | 222 | SEQ ID |
| | | TCTGTC | | | NO: 11 |
| Reverse | Epgn2R | ACGCAACGTTCTGT | | | SEQ ID |
| | | CAAAAG | | | NO: 12 |
| Forward | Epgn3F | TGTATTGAACATGT | Exon3 | 245 | SEQ ID |
| | | ATTATGAAGTTGG | | | NO: 13 |
| Reverse | Epgn3R | CAACTGTGCTGTGT | | | SEQ ID |
| | | GCATCC | | | NO: 14 |
| Forward | Epgn4F | CCTTGTGCCTTAGC | Exon4 | 351 | SEQ ID |
| | | AAATGT | | | NO: 15 |
| Reverse | Epgn4R | AGACACACACATCA | | | SEQ ID |
| | | ACAATAGGG | | | NO: 16 |
| Forward | Epgn5-1F | TCCATCACCATCAA | Exon5_ | 546 | SEQ ID |
| | | CACACAG | 1 | | NO: 17 |
| Reverse | Epgn5-1R | CCCTGAAATTCCAT | | | SEQ ID |
| | | AAGGACAG | | | NO: 18 |
| Forward | Epgn5-2F | TCAATCAAGTCCAA | Exon5_ | 530 | SEQ ID |
| | | GTAATGCC | 2 | | NO: 19 |
| Reverse | Epgn5-2R | ATTCCACATGCTGTT | | | SEQ ID |
| | | GTTCC | | | NO: 20 |
| Forward | Epgn5-3F | TCACACTTGGTATC | Exon5_ | 549 | SEQ ID |
| | | CATCGC | 3 | | NO: 21 |
| Reverse | Epgn5-3R | AAAAGAAATAAGCC | | | SEQ ID |
| | | CATGGTTC | | | NO: 22 |
| Forward | Ereg1F | CTAGTAAGTCCTCG | Exon1 | 319 | SEQ ID |
| | | CGTGCC | | | NO: 23 |
| Reverse | Ereg1R | GTCCTATGACAAAT | | | SEQ ID |
| | | GCACCG | | | NO: 24 |
| Forward | Ereg2F | AACGAAAGTTGCTT | Exon2 | 225 | SEQ ID |
| | | TCCTGG | | | NO: 25 |
| Reverse | Ereg2R | TTCATTGGCATGTA | | | SEQ ID |
| | | CTTATATTGAC | | | NO: 26 |
| Forward | Ereg3F | GCTATACAGCCAGA | Exon3 | 244 | SEQ ID |
| | | GTCCCAG | | | NO: 27 |
| Reverse | Ereg3R | CAGAAGAGGCGAG | | | SEQ ID |
| | | GAAAGTG | | | NO: 28 |
| Forward | Ereg4F | GGTTTCTTATATTGC | Exon4 | 355 | SEQ ID |
| | | AGTGGGG | | | NO: 29 |
| Reverse | Ereg4R | CGCAGATTCCACTT | | | SEQ ID |
| | | TGTTCC | | | NO: 30 |
| Forward | Ereg5-1F | TGTCTGGGCATTTTA | Exon5_ | 612 | SEQ ID |
| | | CATAAGC | 1 | | NO: 31 |
| Reverse | Ereg5-1R | TGTGCCAAGCCATA | | | SEQ ID |
| | | ATTCAG | | | NO: 32 |
| Forward | Ereg5-2F | TGACATTCCTGCGA | Exon5_ | 566 | SEQ ID |
| | | GCTTAG | 2 | | NO: 33 |
| Reverse | Ereg5-2R | CAAGATCTGGTTAG | | | SEQ ID |
| | | CGCCC | | | NO: 34 |
| Forward | Ereg5-3F | TCTGAAGACAAATT | Exon5_ | 598 | SEQ ID |
| | | TGAACCCC | 3 | | NO: 35 |
| Reverse | Ereg5-3R | TTCCATGGGTATGG | | | SEQ ID |
| | | ATAATGAAG | | | NO: 36 |
| Forward | Ereg5-4F | GGTGCCCAGATTCA | Exon5_ | 716 | SEQ ID |
| | | AAATG | 4 | | NO: 37 |
| Reverse | Ereg5-4R | TAGTGCACCAGAAT | | | SEQ ID |
| | | GCCTTG | | | NO: 38 |
| Forward | Ereg5-5F | AAGTGCCAAGGGAC | Exon5_ | 560 | SEQ ID |
| | | CAATC | 5 | | NO: 39 |
| Reverse | Ereg5-5R | TACCCCAAGGTTCT | | | SEQ ID |
| | | TGCATC | | | NO: 40 |
| Forward | Ereg5-6F | GAAAACAGGCGTTT | Exon5_ | 561 | SEQ ID |
| | | TAGATATTGG | 6 | | NO: 41 |
| Reverse | Ereg5-6R | CACGGAGTGGATCT | | | SEQ ID |
| | | ACTGGAC | | | NO: 42 |
| Forward | Ereg5-7F | CACTATACTGAGAA | Exon5_ | 549 | SEQ ID |
| | | CCAAAGCAACC | 7 | | NO: 43 |
| Reverse | Ereg5-7R | AAGTGCTGTTTATG | | | SEQ ID |
| | | CTCCCC | | | NO: 44 |
| Forward | Ereg5-8F | CAAATTTGTCACGT | Exon5_ | 605 | SEQ ID |
| | | TTGGTTC | 8 | | NO: 45 |
| Reverse | Ereg5-8R | CCGACACTACTTTC | | | SEQ ID |
| | | AAGCCC | | | NO: 46 |
| Forward | Areg1F | TCCAGCGGGTCTAT | Exon1 | 387 | SEQ ID |
| | | AAAAGC | | | NO: 47 |
| Reverse | Areg1R | GACGTTTGGGGAAT | | | SEQ ID |
| | | GTCAAG | | | NO: 48 |
| Forward | Areg2F | TGTCTACACAGCAA | Exon2 | 362 | SEQ ID |
| | | TGCTCAC | | | NO: 49 |
| Reverse | Areg2R | CTTTGCAGAGGTAA | | | SEQ ID |
| | | AACCCG | | | NO: 50 |
| Forward | Areg3F | AAAAGAGAACGTGG | Exon3 | 339 | SEQ ID |
| | | GCAGTC | | | NO: 51 |
| Reverse | Areg3R | CCTTGGTTTACACA | | | SEQ ID |
| | | CTGGAAAAG | | | NO: 52 |
| Forward | Areg4F | GTATGTTTGAGATG | Exon4 | 284 | SEQ ID |
| | | CAGAAAACC | | | NO: 53 |
| Reverse | Areg4R | TGACAGTAGAGTCT | | | SEQ ID |
| | | GATAGGAATACTG | | | NO: 54 |
| Forward | Areg5F | GTCACAGGAGGAAA | Exon5 | 283 | SEQ ID |
| | | GTCCCC | | | NO: 55 |
| Reverse | Areg5R | CTTCTTTTGCCTTGA | | | SEQ ID |
| | | GATAAGTG | | | NO: 56 |
| Forward | Areg6F | TGGTGGTTTTCTATA | Exon6 | 462 | SEQ ID |
| | | AGAATATTTAGC | | | NO: 57 |
| Reverse | Areg6R | AAGCCCTTGTAAAT | | | SEQ ID |
| | | GATTGTCC | | | NO: 58 |
| Forward | Btc1F | AAGGTTCACAGGAC | Exon1 | 395 | SEQ ID |
| | | TCCAGC | | | NO: 59 |
| Reverse | Btc1R | AAGTGGTGTGCCTC | | | SEQ ID |
| | | TCCG | | | NO: 60 |
| Forward | Btc2F | AAAAGGATTAAGCA | Exon2 | 232 | SEQ ID |
| | | TTTGGGG | | | NO: 61 |
| Reverse | Btc2R | TGGGTCATGACACT | | | SEQ ID |
| | | AAAAGGAC | | | NO: 62 |
| Forward | Btc3F | TTTCCAATGCTGTCA | Exon3 | 249 | SEQ ID |
| | | AACCC | | | NO: 63 |
| Reverse | Btc3R | GAATCAATGAATAA | | | SEQ ID |
| | | AACGGGG | | | NO: 64 |
| Forward | Btc4F | TGGGAAGAGGAGA | Exon4 | 278 | SEQ ID |
| | | AAAGCAAG | | | NO: 65 |
| Reverse | Btc4R | GAAAACAGTGCTAA | | | SEQ ID |
| | | TTACTTCCAAAG | | | NO: 66 |
| Forward | Btc5F | ATTAGACATGGTGA | Exon5 | 241 | SEQ ID |
| | | AGCCCC | | | NO: 67 |
| Reverse | Btc5R | TTTGCATACTGACA | | | SEQ ID |
| | | GGAACTCAC | | | NO: 68 |
| Forward | Btc6-1F | TGCTGAGCAGACAG | Exon6_ | 541 | SEQ ID |
| | | TCACG | 1 | | NO: 69 |
| Reverse | Btc6-1R | TTGGGCTCATGTGC | | | SEQ ID |
| | | GAG | | | NO: 70 |
| Forward | Btc6-2F | TGCTTTGGTCAGTC | Exon6_ | 582 | SEQ ID |
| | | AACCAG | 2 | | NO: 71 |
| Reverse | Btc6-2R | TTTTCCCACTCTTGT | | | SEQ ID |
| | | GGTCC | | | NO: 72 |
| Forward | Btc6-3F | TTCCCATTGGCTGCT | Exon6_ | 533 | SEQ ID |
| | | GTAAG | 3 | | NO: 73 |
| Reverse | Btc6-3R | CCAACTGAACCACA | | | SEQ ID |
| | | CTTTGC | | | NO: 74 |
| Forward | Btc6-4F | TCCATGAAGTCAAT | Exon6_ | 533 | SEQ ID |
| | | CCTGAAG | 4 | | NO: 75 |
| Reverse | Btc6-4R | GAGGCAGGCAAATC | | | SEQ ID |
| | | TCTGTG | | | NO: 76 |
| Forward | Btc6-5F | TGGTTGGTTGGTTG | Exon6_ | 567 | SEQ ID |
| | | GTTAGG | 5 | | NO: 77 |
| Reverse | Btc6-5R | TGAAGCACAAAAGC | | | SEQ ID |
| | | CAACTC | | | NO: 78 |

Genotyping Mcub mutation of Areg gene

| | | | | |
|---|---|---|---|---|
| Forward | Mcub-1F | CGGTGGAACCAATG | 197 | SEQ ID |
| | | AGAACT | | NO: 79 |
| Reverse | Mcub-1R | CTCACCCTCCTAGC | | SEQ ID |
| | | ATGAGC | | NO: 80 |

| | |
|---|---|
| after HphI digestion | |
| wildtype | 118 bp |
| | +79 bp |
| mutant | 197 bp |

(uncut)

cDNA primers (within Areg exons)

| | | | | |
|---|---|---|---|---|
| Forward | ARwex1F | CGGTGGAACCAATG | 236 | SEQ ID |
| | | AGAACT | | NO: 81 |
| Reverse | ARwex2R | GTCGTAGTCCCCTG | | SEQ ID |
| | | TGGAGA | | NO: 82 |
| Forward | ARwex1F | CGGTGGAACCAATG | 385 | SEQ ID |
| | | AGAACT | | NO: 83 |
| Reverse | ARwex3R | TTGCCTCCCTTTTTC | | SEQ ID |
| | | TTCCT | | NO: 84 |
| Forward | ARwex1F | CGGTGGAACCAATG | 535 | SEQ ID |
| | | AGAACT | | NO: 85 |
| Reverse | ARwex4R | CCACACCGTTCACC | | SEQ ID |
| | | AAAGTA | | NO: 86 |
| Forward | ARwex1F2 | GTTGCTGCAGAGAC | 265 | SEQ ID |
| | | CGAGAC | | NO: 87 |
| Reverse | ARwex2R | GTCGTAGTCCCCTG | | SEQ ID |
| | | TGGAGA | | NO: 88 |
| Forward | ARwex2F | CTGGCAGTGAACTC | 331 | SEQ ID |
| | | TCCACA | | NO: 89 |
| Reverse | ARwex4R | CCACACCGTTCACC | | SEQ ID |
| | | AAAGTA | | NO: 90 |

Those skilled in the art will recognize, or be able to ascertain using no more than routine experimentation, many equivalents to the specific embodiments of the invention described herein. Such equivalents are intended to be encompassed by the following claims.

1. A method for identifying a compound that modulates activity of an iRhom polypeptide comprising:
contacting a cell expressing an iRhom polypeptide with a test compound; and
determining an effect of the test compound on activity of the iRhom polypeptide.

2. The method of claim 1, wherein determining the effect of the test compound on activity of the iRhom polypeptide comprises determining stability of the iRhom polypeptide compared to an appropriate control.

3. The method of claim 1, wherein determining the effect of the test compound on activity of the iRhom polypeptide comprises determining a level of secretion of a physiological target of the iRhom polypeptide compared to an appropriate control.

4. The method of claim 1, wherein determining the effect of the test compound on activity of the iRhom polypeptide comprises determining a level of EGFR activity relative to an appropriate control.

5. The method of claim 1, wherein determining the effect of the test compound on activity of the iRhom polypeptide comprises determining a level of a soluble EGFR ligand compared to an appropriate control.

6. The method of claim 1, wherein activity of the iRhom polypeptide is proteolytic activity to cleave a substrate.

7. The method of claim 1, wherein determining the effect of the test compound on activity of the iRhom polypeptide comprises detecting proteolytic activity of the iRhom polypeptide to proteolytically cleave a substrate compared to an appropriate control.

8. The method of claim 2, wherein a decrease in stability of the iRhom polypeptide indicates that the compound decreases proteolytic activity of the iRhom polypeptide, and wherein an increase in stability of the iRhom polypeptide indicates that the compound increases proteolytic activity of the iRhom polypeptide.

9. The method of claim 3, wherein an increase in secretion of the physiological target of the iRhom polypeptide indicates that the compound increases proteolytic activity of the iRhom polypeptide, and wherein a decrease in secretion of the physiological target of the iRhom polypeptide indicates that the compound inhibits proteolytic activity of the iRhom polypeptide.

10. The method of claim 4, wherein an increase in EGFR activity indicates that the compound increases proteolytic activity of the iRhom polypeptide to proteolytically cleave a substrate, and wherein a decrease in EGFR activity indicates that the compound inhibits proteolytic activity of the iRhom polypeptide.

11. The method of claim 5, wherein an increase in the soluble EGFR ligand indicates that the compound increases proteolytic activity of the iRhom polypeptide, and wherein a decrease in a soluble EGFR ligand indicates that the compound inhibits proteolytic activity of the iRhom polypeptide.

12. The method of claim 1, wherein a decrease in proteolytic activity of the iRhom polypeptide indicates that the compound is capable of one or more of: reducing tumor growth, reducing tumor progression, treatment of cancer and promoting hair growth in a subject, and an increase in proteolytic activity of the iRhom polypeptide indicates that the compound is capable of accelerating wound healing in a subject.

13. The method of claim 1, wherein the cell expresses an EGFR ligand.

14. The method of claim 6, wherein the substrate is an EGFR ligand or an EGF-like substrate.

15. The method of claim 3, wherein the physiological target is an EGFR ligand.

16. The method of claim 5, wherein the EGFR ligand is selected from the group consisting of: AREG, HB-EGF, TGFα and EPGN.

17. The method of claim 1, wherein the cell is in vitro.

18. The method of claim 1, wherein the iRhom polypeptide is iRhom1 or iRhom2.

19. The method of claim 1, wherein the iRhom polypeptide is a human or mouse iRhom.

20. The method of claim 1, wherein the test compound is selected from the group consisting of: a small molecule, a peptide, a polypeptide decoy, an miRNA molecule, an siRNA molecule, an shRNA molecule, a dsRNA molecule, an antisense molecule, a ribozyme specific for Rhbdf2; or a polynucleotide encoding an miRNA, siRNA, shRNA, dsRNA; a combination of any two or more thereof; and a biological equivalent of any one or more thereof.

21. (canceled)
